(19) United States
(12) Patent Application Publication
(10) Pub. No.: US 2025/0236900 A1
(43) Pub. Date: Jul. 24, 2025

(54) Method for Producing Double-Stranded DNA Molecules Having Reduced Sequence Errors

(71) Applicant: Moderna Enzymatics Co., Ltd., Tokyo (JP)

(72) Inventors: Masayuki Suetsugu, Tokyo (JP); Takuya Koyama, Tokyo (JP); Hayato Ishitobi, Tokyo (JP)

(21) Appl. No.: 18/848,233

(22) PCT Filed: Mar. 31, 2023
(86) PCT No.: PCT/JP2023/013486

§ 371 Date: Sep. 18, 2024

(30) Foreign Application Priority Data
Mar. 31, 2022 (JP) 2022-060087

(51) Int. Cl. C12P 19/34; C12N 9/22

(52) U.S. Cl. CPC C12P 19/34; C12N 9/22

(57) ABSTRACT

Provided is a method to remove double-stranded DNA with sequence errors, which can occur in various stages of DNA production such as chemical synthesis, hybridization, and amplification, from double-stranded DNA without sequence errors, thereby providing double-stranded DNA with a low proportion of sequence errors. Specifically, this invention is a method for producing double-stranded DNA, which includes: (1) providing a double-stranded DNA mixture containing double-stranded DNA with sequence errors and double-stranded DNA without sequence errors; (2) adding a mismatch repair-related enzyme group to the double-stranded DNA mixture, where the mismatch repair-related enzyme group includes MutS and MutL, or MutS and single-strand specific exonuclease; and (3) subjecting the double-stranded DNA mixture to a double-stranded DNA amplification reaction.

### TECHNICAL FIELD

The present invention relates to methods for removing double-stranded DNA with sequence errors from the amplified products of double-stranded DNA amplification reactions and methods for amplifying double-stranded DNA without producing double-stranded DNA with sequence errors.

This application claims priority based on Japanese Patent Application No. 2022-060087 filed in Japan on Mar. 31, 2022, the content of which is incorporated herein by reference.

### BACKGROUND ART

Chemically synthesized oligo DNA contains sequence errors that differ from the designed sequence at a certain frequency. This is problematic when producing double-stranded DNA using oligo DNA. The longer the target double-stranded DNA, the higher the probability of including sequence errors. Even the highest quality oligonucleotides have an error rate of less than one per 750 bases, meaning that in the case of synthetic double-stranded DNA longer than 1.5 kb, DNA construct that deviates from the intended sequence can be generated (Non-Patent Literature 1). Additionally, when using homologous sequences to assemble double-stranded DNAs, unintended DNA constructs can arise from incorrect assembly of similar sequences, which is problematic. Furthermore, replication errors occurring at a certain frequency during in vitro DNA replication and amplification also pose a problem. Thus, DNA constructs deviating from the intended sequence can arise at multiple stages of DNA production, including synthesis, assembly, and amplification.

Enzymatic error correction methods using enzymes that cleave and rejoin distorted DNA helices caused by errors exist as ways to reduce sequence errors through error correction or removal. Compared to size exclusion purification methods that sort DNA by size post-synthesis or methods that repair errors based on hybridization efficiency differences, enzymatic methods are usually lower in cost and more efficient (Non-Patent Literature 2).

Commercial kits using the activity of nucleases that specifically cleave mutation sites for detecting mutations in DNA or genes are also available. Examples of such kits include the Surveyor (registered trademark) Mutation Detection Kits (Integrated DNA Technologies) using celery-derived CEL family nucleases (Non-Patent Literatures 3 and 4), T7 Endonuclease I (New England Biolabs) using T7 phage-derived nucleases (Non-Patent Literature 5), CorrectASE (formerly ErrASE Error Correction Kit) (Non-Patent Literatures 3 and 5), EndoMS (Non-Patent Literatures 6 and 7) and Mismatch Endonuclease I (New England Biolabs) that recognize and cleave double-stranded DNA with mismatch base pairs (T: T, G: G, T: G).

Living organisms have a mismatch repair (MMR) system to correct mismatches occurring during DNA replication. MutS, a mismatch-binding protein, is a crucial component of the MMR system, recognizing and binding to various types of sequence errors in DNA, specifically almost all single-base mismatches and 1-4 base insertions or deletions (Non-Patent Literatures 8 to 10), functioning in the accuracy of DNA replication in both eukaryotes and prokaryotes.

Methods using MutS for mismatch reduction have also been explored. As a method for detecting mutations in gene fragments amplified by PCR, cleaving the double-stranded “GATC” using MutS, MutL, MutH endonuclease and ATP for the sequence denatured and reannealed by PCR, mimicking the methyl-directed mismatch repair mechanism in E. coli (Non-Patent Literature 11), followed by size selection to isolate uncut products and detect and reduce mismatches (Non-Patent Literature 12) has been disclosed. Methods for reacting PCR products with MutS derived from the thermophilic bacterium Thermus aquaticus and separating the reaction products by electrophoresis to obtain products with reduced errors have also been disclosed (Non-Patent Literature 13). Additionally, a method using the characteristics of MutS derived from Aquifex aeolicus to suppress errors during nucleic acid amplification reactions in PCR has been disclosed (Patent Literature 1).

### PRIOR ART LITERATURES

### Patent Literatures

Patent Literature 1: International Publication No. WO 2013/175815

### Non-Patent Literatures

Non-Patent Literature 1: Katz et al., Journal of Industrial Microbiology & Biotechnology 2018 vol. 45 p.449-461.
Non-Patent Literature 2: Zhang et al., ACS Synthetic Biology 2020 vol. 9 p.940-952.
Non-Patent Literature 3: Quan J. et al., Nature Biotechnology 2011 vol. 29 p449-452.
Non-Patent Literature 4: Saaem et al., Nucleic Acids Research 2012 vol. 40 No. 3 e23.
Non-Patent Literature 5: Sequeira et al., Molecular Biotechnology 2016 vol. 58 p.573-584.
Non-Patent Literature 6: Ishino et al., Nucleic Acids Research 2016 vol. 44 No. 7 p.2977-2986.
Non-Patent Literature 7: Takemoto et al., Nucleic Acids Research 2018 vol. 46 No. 12 p.6152-6165.
Non-Patent Literature 8: Harfe and Jinks-Robertson, Annual Review of Genetics 2000 vol. 34 p.359-399.
Non-Patent Literature 9: Iyer et al., Chemical Reviews 2006 vol. 106 p.302-323.
Non-Patent Literature 10: Modrich, Annual Review of Genetics 1991 vol. 25 p.229-253.
Non-Patent Literature 11: Smith and Modrich, Proceedings of the National Academy of Sciences of the United States of America 1996 vol. 93 p.4374-4379.
Non-Patent Literature 12: Smith and Modrich, Proceedings of the National Academy of Sciences of the United States of America 1997 vol. 94 p.6847-6850.
Non-Patent Literature 13: Carr et al., Nucleic Acids Research 2004 vol. 32 No. 20 e162.

### SUMMARY OF INVENTION

### Problem to be Solved by the Invention

Patent Literature 1 discloses that using MutS in PCR reactions can suppress non-specific amplification and sequence errors during amplification. However, it requires MutS derived from specific microorganisms active at high-temperature and does not show effects when using other enzymes such as MutL from specific microorganisms. Methods described in Non-Patent Literatures 8 and 9 disclose that combining other enzymes with MutS can remove sequence errors that have occurred, but they require laborious steps such as size selection and purification of the reaction products.

In view of the above background, the present invention aims to provide a method for efficiently removing double-stranded DNA with unintended sequence errors from double-stranded DNA without sequence errors using an enzymatic and simple method, thereby producing double-stranded DNA without sequence errors during multiple stages of DNA production such as chemical synthesis, hybridization, and amplification.

### Means for Solving the Problem

The inventors of the present invention conducted intensive research and found that using a specific combination of enzymes on double-stranded DNA with sequence errors before and/or during amplification can remove double-stranded DNA molecules with sequence errors from the amplified products without subsequent purification steps. The inventors discovered that double-stranded DNA with various sequence errors, such as sequence errors occurring during oligo DNA synthesis, sequence errors in double-stranded DNA obtained by annealing single-stranded DNA and a part or all of its complementary strand, and sequence errors occurring during double-stranded DNA amplification, can be removed similarly, selectively amplifying double-stranded DNA without sequence errors, leading to the completion of the present invention.

Thus, the methods according to the present invention are as follows:

[1] A method for producing double-stranded DNA, comprising:
(1) providing a double-stranded DNA mixture containing double-stranded DNA with sequence error and double-stranded DNA without sequence error;
(2) adding a mismatch repair-related enzyme group to the double-stranded DNA mixture, wherein the mismatch repair-related enzyme group comprising MutS and MutL; and (3) subjecting the double-stranded DNA mixture to a double-stranded DNA amplification reaction.

[2] The method according to [1], wherein the mismatch repair-related enzyme group further comprises enzymes selected from MutH, UvrD, and a combination of UvrD and a single strand-specific exonuclease.

[3] The method according to [1] or [2], wherein said (2) comprises acting the mismatch repair-related enzyme group on double-stranded DNA with sequence error in the double-stranded DNA mixture.

[4] The method according to any one of [1] to [3], wherein the mismatch repair-related enzyme group further comprises UvrD and single strand-specific exonuclease, and the single strand-specific exonuclease is ExoVII.

[5] The method according to [1] or [2], wherein the double-stranded DNA amplification reaction of said (3) is a cell-free amplification reaction, and comprising acting the mismatch repair-related enzyme group on the double-stranded DNA with sequence error in the double-stranded DNA mixture.

[6] The method according to [5], wherein the mismatch repair-related enzyme group further comprises MutH.

[7] A method for producing double-stranded DNA, comprising:
(1) providing a double-stranded DNA mixture containing double-stranded DNA with sequence error and double-stranded DNA without sequence error;
(2) adding a mismatch repair-related enzyme group to the double-stranded DNA mixture, wherein the mismatch repair-related enzyme group comprising MutS and a single strand-specific exonuclease; and
(3) subjecting the double-stranded DNA mixture to a double-stranded DNA amplification reaction, wherein the double-stranded DNA amplification reaction of said (3) is a cell-free amplification reaction, and comprising acting the mismatch repair-related enzyme group on the double-stranded DNA with sequence error in the double-stranded DNA mixture. [8] The method according to [7], wherein the mismatch repair-related enzyme group further comprises one or more enzymes selected from MutL and MutH.

[9] The method according to [7] or [8], wherein the single strand-specific exonuclease is exonuclease I.

[10] The method according to any one of [1] to [9], wherein the amplification reaction is conducted at a temperature of 65° C. or lower.

[11] The method according to any one of [1] to [10], wherein said (1) comprises:
obtaining the double-stranded DNA with sequence error by mis-hybridizing a part or all of the single strand portions in at least one of the combinations selected from:
   a combination of single-stranded DNA and its complementary strand single-stranded DNA,
   a combination of double-stranded DNA with a single-stranded portion and single-stranded DNA with a sequence complementary to at least a part of the single-stranded portion, and
   a combination of double-stranded DNA with a single-stranded portion and double-stranded DNA with a single-stranded portion having a sequence complementary to at least a part of the single-stranded portion,
      or
obtaining the double-stranded DNA with sequence error by hybridizing a part or all of the single strand portions in at least one of the combinations selected from:
   a combination of single-stranded DNA and its complementary strand single-stranded DNA, wherein at least one of the single-stranded DNA has sequence error,
   a combination of double-stranded DNA with a single-stranded portion and single-stranded DNA with a sequence complementary to at least a part of the single-stranded portion, wherein at least one of the double-stranded DNA and the single-stranded DNA has sequence error, and
   a combination of double-stranded DNA with a single-stranded portion and double-stranded DNA with a single-stranded portion having a sequence complementary to at least a part of the single-stranded portion, wherein at least one of the DNAs has sequence error.

[12] A method for producing double-stranded DNA using a double-stranded DNA amplification reaction, comprising:
subjecting a reaction solution containing mismatch repair-related enzyme group and double-stranded DNA to the double-stranded DNA amplification reaction, wherein
   the mismatch repair-related enzyme group comprises: MutS; and MutL and/or single strand-specific exonuclease, and
   the double-stranded DNA amplification reaction is a cell-free amplification reaction conducted at a temperature of 80° C. or lower.

[13] The method according to [12], wherein the mismatch repair-related enzyme group further comprises MutH.

[14] The method according to or [13], wherein the double-stranded DNA subjected to the amplification reaction is double-stranded DNA obtained by hybridizing a part or all of the single-stranded portion of a combination selected from:
a combination of single-stranded DNA and its complementary strand single-stranded DNA,
a combination of double-stranded DNA with a single-stranded portion and double-stranded DNA with single-stranded portion with a sequence complementary to at least a part of said single-stranded portion, and
a combination of double-stranded DNA with a single-stranded portion and double-stranded DNA with a sequence complementary to at least a part of the single-stranded portion.

[15] The method according to any one of [1] to [14], wherein the double-stranded DNA subjected to the amplification reaction is circular double-stranded DNA having a replication origin sequence capable of binding to an enzyme with DnaA activity, and
the double-stranded DNA amplification reaction is an RCR amplification reaction.

[16] A double-stranded DNA obtained by the method according to any one of [1] to [15].

[17] A kit for producing circular double-stranded DNA, comprising:
MutS,
MutL,
UvrD,
single strand-specific exonuclease,
a first enzyme group that catalyzes replication of circular DNA;
a second enzyme group that catalyzes an Okazaki fragment maturation and
synthesizes two sister circular DNAs constituting a catenane; and
a third enzyme group that catalyzes a separation of two sister circular DNAs.

[18] A kit for producing circular double-stranded DNA, comprising:
MutS.
MutL,
MutH and/or single strand-specific exonuclease,
a first group of enzymes catalyzing the replication of circular DNA,
a second group of enzymes catalyzing Okazaki fragment joining reactions to synthesize
two sister circular DNAs forming a catenane, and
a third group of enzymes catalyzing the separation of the two sister circular DNAs.

[19] A kit for producing circular double-stranded DNA, comprising:
MutS,
single strand-specific exonuclease,
a first enzyme group that catalyzes replication of circular DNA,
a second enzyme group that catalyzes an Okazaki fragment maturation and synthesizes
two sister circular DNAs constituting a catenane, and
a third enzyme group that catalyzes a separation of two sister circular DNAs.

### Effect of the Invention

According to the method of the present invention, it is possible to provide a method for producing double-stranded DNA with a reduced proportion of double-stranded DNA molecules with sequence errors by removing double-stranded DNA with sequence errors generated in multiple stages of DNA production, such as chemical synthesis, hybridization, and amplification of DNA.

### BRIEF DESCRIPTION OF DRAWINGS

FIG. 1A is a schematic diagram illustrating the method for distinguishing between DNA with and without mismatches by treating with cleaving enzymes after RCR amplification.

FIG. 1B shows the diagram of electrophoresis results of reaction products using various mismatch repair-related enzyme groups, after RCR amplification and restriction enzyme cleavage in Example 1. FIG. 1C is a graph quantifying the ratio (1-cut ratio) of the band intensity of the 2.6 kb fragment derived from DNA with mismatches relative to the total band intensity from the electrophoresis results in FIG. 1B.

FIG. 2 shows graphs examining the effects of mismatch repair-related enzyme groups at various conditions of reaction times and temperatures and the effects of the mismatch repair-related enzyme group in one-step in Example 2.

FIG. 3 shows graphs examining the effects of mismatch repair-related enzyme groups on various sequence errors in Example 3.

FIG. 4A is a schematic diagram illustrating each assembly reaction and the action of single strand-specific exonuclease in Example 4. FIG. 4B shows graphs examining the effects of mismatch repair-related enzyme group SLD and single strand-specific exonuclease in different assembly reactions in Example 4.

FIG. 5A shows the results of examining the effects of commercial Mismatch Endonuclease I and mismatch repair-related enzyme group SLDE on various sequence errors in Example 5. FIG. 5B shows the results of examining the effects of commercial T7 Endonuclease I and mismatch repair-related enzyme group SLDE on various sequence errors in Example 5.

FIG. 6A is a schematic diagram of the experiment in Example 6. FIG. 6B is a schematic diagram of the data processing in the experiment in Example 6. FIG. 6C shows the results of examining the effects of mismatch repair-related enzyme group SLD on circular DNA assembly products with artificially introduced mismatches using next-generation sequencing (NGS) in Example 6. FIG. 6D shows the results of examining the effects of mismatch repair-related enzyme group SLDE on synthesis errors of oligo DNA without introduced mismatches using NGS in Example 6.

FIG. 7A is a schematic diagram of the experiment in Example 7. FIG. 7B shows the results of confirming the error removal effect on synthetic genes from oligo DNA by measuring the number of E. coli colonies with mutant genes after transformation with synthetic genes in Example 7.

FIG. 8A is a schematic diagram of the experiment in Example 8. FIG. 8B shows the results of confirming the error removal effect on synthetic genes from oligo DNA (Eurofins PAGE-Oligo) by measuring the number of E. coli colonies with mutant genes after transformation with synthetic genes in Example 8. FIG. 8C shows the results of confirming the error removal effect on synthetic genes from oligo DNA (IDT oPools) by measuring the number of E. coli colonies with mutant genes after transformation with synthetic genes in Example 8.

FIG. 9A is a schematic diagram of the experiment in Example 9 and the possible actions of the mismatch repair-related enzyme group. FIG. 9B shows the results of confirming the error removal effect on replication errors occurring during the RCR amplification reaction by measuring the number of E. coli colonies with mutant genes after transformation with the amplified gene in Example 9.

FIG. 10 shows the results of examining the effects of various mismatch repair-related enzyme groups (with or without ExoI) added during the RCR amplification reaction in Example 10.

FIG. 11 shows the results of examining whether the error removal reaction by mismatch repair-related enzyme groups SLD or SLDE is effective in a system where E. coli transformation is performed directly without in vitro DNA amplification in Example 11.

FIG. 12 shows the results of examining the error removal effect of mismatch repair-related enzyme group SLDE on the amplification product after PCR amplification and the error removal effect on the amplification product in Example 12.

FIG. 13 shows schematic diagrams of various sequence errors on which the effects of mismatch repair-related enzyme groups are confirmed in the examples.

### DETAILED DESCRIPTION OF THE EMBODIMENTS

In the present invention and this specification, “double-stranded DNA” refers, unless otherwise specified, to a collection of double-stranded DNA molecules. More specifically, it refers to a collection of DNA having at least a part of its molecular structure in double-stranded form. Thus, “double-stranded DNA” in the context of this invention includes not only DNA that is double-stranded throughout its entire length (typically, both sides blunt-ended double-stranded DNA), but also DNA where one single-stranded DNA molecule hybridizes with another single-stranded DNA molecule that is complementary only in part, resulting in DNA that has a single strand structure on 3′ end and/or 5′ end (3′ overhang and/or a 5′ overhang). Furthermore, DNA that involves three or more single-stranded DNAs hybridizing together, possessing gap or nick, is also considered “double-stranded DNA” within the scope of this invention. As an example of double-stranded DNA composed of three or more single strands, DNA in which two or more single-stranded DNA strands hybridize to one single-stranded DNA strand at different sites to form a double-stranded structure can be cited.

In the present invention and this specification, a “gap” refers to a state in double-stranded DNA where one or more contiguous nucleotides do not hybridize and remain in a single-stranded form. A “nick” refers to a condition where the phosphodiester bond between adjacent nucleotides on one of the strands that make up the double-stranded DNA is cleaved.

In the present invention and this specification, “sequence error” refers to a modification where one or more bases in the target sequence are substituted (substitution of a base other than the base that forms a normal base pair, such as A with T or U, G with C, etc.), inserted, or deleted. Here, “target sequence” refers to the sequence of the double-stranded DNA intended to be produced by the amplification reaction. If the target double-stranded DNA is designed to have a predetermined sequence, “target sequence” refers to the sequence as designed.

In parts of the double-stranded structure where sequence errors exist, normal base pairs are not formed. In this specification, “double-stranded DNA with sequence error” refers to DNA in which a part of its double-stranded structure does not form normal base pair. Conversely, “double-stranded DNA without sequence error” refers to DNA in which all bases constituting the double-stranded parts form normal base pairs.

Additionally, in a part of the double-stranded DNA structure where corresponding base is present but does not form normal base pair because they are not bases that form normal base pair, the part is referred to as “mismatch.” When a sequence error is caused by a single-base substitution, the part with the sequence error correspond to a mismatch region.

The specific embodiments for implementing this invention are described in detail below (hereafter also referred to as “the present embodiment”), but the invention is not limited to these.

The present embodiment relates to a method for producing double-stranded DNA, comprising:
(1) providing a double-stranded DNA mixture containing double-stranded DNA with sequence error and double-stranded DNA without sequence error;
(2) adding a mismatch repair-related enzyme group to the double-stranded DNA mixture, wherein the mismatch repair-related enzyme group comprising MutS and MutL; and
(3) subjecting the double-stranded DNA mixture to a double-stranded DNA amplification reaction.

In another aspect, the present embodiment provides a method for producing double-stranded DNA, comprising:
(1) providing a double-stranded DNA mixture containing double-stranded DNA with sequence error and double-stranded DNA without sequence error;
(2) adding a mismatch repair-related enzyme group to the double-stranded DNA mixture, wherein the mismatch repair-related enzyme group comprising MutS and a single strand-specific exonuclease; and
(3) subjecting the double-stranded DNA mixture to a double-stranded DNA amplification reaction,
wherein the double-stranded DNA amplification reaction of (3) is a cell-free amplification reaction, and comprising acting the mismatch repair-related enzyme group on the double-stranded DNA with sequence error in the double-stranded DNA mixture.

In the present embodiment, the mismatch repair-related enzyme group is reacted on the double-stranded DNA with sequence errors. This results in selective amplification of the double-stranded DNA without sequence errors during the amplification reaction, producing amplified products that are either free from or contain a very low proportion of double-stranded DNA with sequence errors.

As the target for removal in the present embodiment, the double-stranded DNA with sequence errors refers to any double-stranded DNA where at least one of the strands forming the double-stranded structure contains sequence error, and normal base pairs are not formed at the error sites. For example, double-stranded DNA may have sequence error on only one of the strands forming the double strand or on both strands.

The types of sequence error in the double-stranded DNA targeted for removal include substitutions, insertions, and deletions of bases. The sequence error may be a combination of multiple such errors. If there are multiple sequence errors, they do not need to be consolidated on only one strand; for instance, if strands A and B hybridize to form a double strand with two sequence error sites (sites X and Y), site X could have a base substitution on strand A, and site Y could have a different base substitution or a base deletion on strand B. In one embodiment, it is preferable for the double-stranded DNA with sequence errors to have one strand with the intended sequence and the other strand with sequence error selected from base substitution, base insertion, and base deletion. Examples of specific sequence error include mismatch-forming base substitutions, wherein the mismatch is selected from AA, AG, AC, CT, CC, GT, GG, and TT; base deletions or insertions of A, C, G, or T, where T can also be U. Additionally, if the intended base sequence is designed to form a specific artificial base pair, a base substitution to another base or artificial base that does not pair may also be considered a sequence error.

The number of sequence errors per double-stranded DNA molecule is not specifically limited as long as the single-stranded DNA and its complementary strand hybridize to form double-stranded DNA. The number of errors can vary depending on the stringency of the environmental conditions in which the double-stranded DNA molecules exist. In one embodiment, the number of sequence errors is less than ten per 100 base pairs, preferably less than eight, more preferably less than five, and most preferably less than three per 100 base pairs.

The proportion of double-stranded DNA with sequence errors in the double-stranded DNA mixture is not specifically limited; for example, it can be less than or equal to 95%, 90%, 80%, 75%, 70%, 60%, 50%, 40%, 30%, 25%, 20%, 10%, 5%, 3%, 2%, 1%, 0.6%, 0.5%, 0.4%, 0.3%, etc. According to the present invention, it is possible to produce double-stranded DNA with a lower proportion of sequence errors, even if the proportion of double-stranded DNA with sequence errors is low. In one embodiment, the proportion of double-stranded DNA with sequence errors in the double-stranded DNA mixture can be less than or equal to 20%, 10%, 5%, 3%, 2%, 1%, 0.6%, 0.5%, 0.4%, 0.3%, 0.2%, 0.1%, 0.05%, 0.02%, etc.

Examples of double-stranded DNA molecules with sequence errors include a double-stranded DNA obtained by hybridizing a part or all of the single-stranded portions in at least one of the combinations selected from:
a combination of single-stranded DNA and its complementary strand single-stranded DNA, wherein a sequence error is present in either or both single strands;
a combination of double-stranded DNA with a single-stranded portion and single-stranded DNA with a sequence complementary to at least a part of the single-stranded portion, wherein a sequence error is present in the double-stranded structure portion of the double-stranded DNA, or in either or both of the single-stranded portion of the double-stranded DNA and the single-stranded DNA; and
a combination of double-stranded DNA with a single-stranded portion and double-stranded DNA with a single-stranded portion having a sequence complementary to at least a part of the single-stranded portion, wherein a sequence error is present in either or both of the double-stranded DNAs (the sequence error in these double-stranded DNAs can be present in either the single-stranded portion or the double-stranded structure portion). The double-stranded DNA molecules with sequence error can also be double-stranded DNA obtained by hybridizing a part or all of the single-stranded portion of two or more single strands or a double strand with a single-stranded portion. The double-stranded DNA molecule with sequence error can also be double-stranded DNA composed of three or more DNA molecules by combining two or more such combinations. In the present specification: hybridization of a part of a single-stranded portion of single-stranded DNA with its complementary single-stranded DNA; hybridization of a double-stranded DNA with a single-stranded portion and a part of a single-stranded DNA that is complementary to at least a part of the single-stranded portion; or hybridization of a double-stranded DNA with a single-stranded portion and a part of a double-stranded DNA with a single-stranded portion that is complementary to the single-stranded portions; is also referred to as assembly.

Double-stranded DNA molecules with sequence errors can also be double-stranded DNA molecules that have replication errors generated during the amplification reaction.

An example of a double-stranded DNA molecule with sequence error includes a double-stranded DNA with sequence error on at least one of the double strands, preferably on one of the double strands, obtained by mis-hybridizing a part or all of the single strand portions in at least one of the combination selected from:
a combination of single-stranded DNA and its complementary single-stranded DNA;
a combination of double-stranded DNA with a single-stranded portion and single-stranded DNA with a sequence complementary to at least a part of the single-stranded portion; and
a combination of double-stranded DNA with a single-stranded portion and double-stranded DNA with a single-stranded portion having a s sequence complementary to at least a part of the single-stranded portion. A double-stranded DNA molecule with sequence error may be a double-stranded DNA obtained by combining two or more of these combinations, so that three or more DNA molecules are mis-hybridized with a part or all of their assembly portions.

The term “mis-hybridization” refers to instances in the combinations mentioned above where, during the hybridization process of a part or all of the single-stranded portion, a part of the single stranded portion hybridize incorrectly with similar sequences rather than with the intended complementary sequences, or where parts of the single-stranded portion sift during hybridization from their complementary sequences, resulting in the formation of unintended double-stranded DNA. Mis-hybridization also includes instances where single strands or a single stranded portion of double-stranded DNA hybridize to a sequence similar to the complementary sequence within the double strand, forming a three-stranded structure known as a D-loop.

Examples of single-stranded DNA with sequence error or double-stranded DNA with single-stranded portion that have sequence error include: single-stranded DNA with sequence error generated during chemical synthesis of the single-stranded DNA (oligonucleotide); single-stranded DNA synthesized based on a template DNA or RNA that has introduced sequence errors; and double-stranded DNA that has replication errors during amplification where parts or all of the DNA are rendered single-stranded due to denaturation or enzymatic treatment.

Next, the mismatch repair-related enzyme group is added to the double-stranded DNA mixture. This allows the mismatch repair-related enzyme group to act on the double-stranded DNA molecule with sequence error in the mixture. The mismatch repair-related enzyme group can be added to the double-stranded DNA mixture before it is subjected to the double-stranded DNA amplification reaction, allowing the enzyme group to act on the double-stranded DNA molecule with sequence error before starting the amplification reaction.

The action of the mismatch repair-related enzyme group on the double-stranded DNA molecule with sequence error also occurs during the amplification reaction. Therefore, the double-stranded DNA mixture with the added mismatch repair-related enzyme group can be immediately subjected to the double-stranded DNA amplification reaction. Alternatively, the double-stranded DNA mixture can be added to a mixed reaction solution that already contains the mismatch repair-related enzyme group in the double-stranded DNA amplification reaction solution before starting the amplification reaction, or the mismatch repair-related enzyme group can be added to the amplification reaction solution containing the double-stranded DNA mixture after starting the double-stranded DNA amplification reaction, allowing the enzyme group to act on the double-stranded DNA molecule with sequence error during the double-stranded DNA amplification reaction.

Acting the mismatch repair-related enzyme group on double-stranded DNA molecule with sequence error refers to s: the recognition of sequence error by MutS and interaction of MutL with MutS; or recognition of sequence error by MutS and hydrolysis by a single strand-specific exonuclease. If the mismatch repair-related enzyme group includes additional enzymes, these enzymes also perform activities described below in conjunction with these actions. In one embodiment, the action of the mismatch repair-related enzyme group on the sequence error in double-stranded DNA molecules may involve: recognition of sequence error; recognition and hydrolysis of sequence error; recognition of sequence error and cleavage of the double-stranded DNA molecule or insertion of nick; recognition of sequence error and splitting the double-stranded DNA molecule; as well as recognition of sequence error, cleavage of the double-stranded DNA molecule or insertion of nick and splitting the double-stranded DNA molecules, optionally including the degradation of single-stranded DNA molecules from the nick or splitting ends.

As part of the mismatch repair-related enzyme group, enzymes such as MutS and MutL are included, and preferably further including enzyme(s) selected from: MutH; UvrD; and UvrD and a single-strand specific exonuclease. Furthermore, the mismatch repair-related enzyme group may include MutS and a single-strand specific exonuclease, and preferably further include enzyme(s) selected from MutL and MutH. In one embodiment, the mismatch repair-related enzyme group includes MutS, MutL, and UvrD. In one embodiment, the mismatch repair-related enzyme group includes MutS, MutL, and MutH. In one embodiment,, the mismatch repair-related enzyme group includes MutS, MutL, UvrD, and a single-strand specific exonuclease. In one embodiment, the mismatch repair-related enzyme group includes MutS and a single-strand specific exonuclease. In one embodiment, embodiment, it includes MutS, a single-strand specific exonuclease, and MutL. In one embodiment, the mismatch repair-related enzyme group includes MutS, a single-strand specific exonuclease, MutL, and MutH.

MutS can be derived from any organism, as far as it recognizes and binds to mismatched DNA base pairs. Kown MutS such as MutS derived from various bacteria including thermophilic bacteria and E. coli, as well as known family proteins thereof can be used. Mut S wherein mutations have been introduced or amino acids have been modified in known amino acid sequence of MutS can also be used as long as they retain the ability to recognize and bind to mismatched DNA base pairs. In one embodiment, MutS from E. coli and thermophilic bacteria or Mut S wherein mutations have been introduced or amino acids have been modified in such MutS can be used, with E. coli-derived MutS being preferable, and E. coli-derived wild type MutS being particularly prefarable. MutS may be included in the reaction, when acting on the sequence errors, at concentrations in the range of 10 nM to 500 nM, preferably between 10 nM and 300 nM, 30 nM to 500 nM, 30 nM to 300 nM, 30 nM to 200 nM, and 50 nM to 300 nM, and more preferably between 100 nM to 300 nM, but not limited to these.

MutL is a protein that interacts with MutS, which has recognized mismatched base pairs, to form a complex. In some species, MutL has endonuclease activity, but MutL from E. coli does not have endonuclease activity. MutL can be used regardless of the presence or absence of endonuclease activity, and known MutL such as MutL from E. coli and its related species, or their family proteins, can be used. Even if mutations are introduced into the amino acid sequence of known MutL or if the amino acids are modified in the amino acid sequence of known MutL, it can be used as long as it has the same function as MutL from E. coli, which interacts with MutS that has recognized mismatched base pairs. In one embodiment, MutL from E. coli is preferably used. MutL may be included in the range of 30 nM to 1000 nM when acting on sequence errors, preferably in the range of 30 nM to 500 nM, 30 nM to 300 nM, 30 nM to 200 nM, 30 nM to 500 nM, 50 nM to 400 nM, 50 nM to 300 nM, 50 nM to 200 nM, 50 nM to 150 nM, 100 nM to 500 nM, 100 nM to 400 nM, and more preferably in the range of 100 nM to 300 nM, but it is not limited to these ranges.

MutH is a protein that, in prokaryotes such as E. coli, is activated by MutS and MutL and introduces nicks into unmethylated DNA (if both strands of double-stranded DNA are unmethylated, it cleaves the double strand). Known MutH such as MutH derived from E. coli and other sources can be used. Even if mutations are introduced into the amino acid sequence of known MutH or if the amino acids are modified, it can be used as long as it has the above activity. In one embodiment, MutH from E. coli is preferably used. MutH may be included in the range of 10 nM to 500 nM when acting on sequence errors, preferably in the range of 10 nM to 300 nM, 10 nM to 200 nM, 30 nM to 500 nM, 30 nM to 300 nM, 30 nM to 200 nM, 50 nM to 300 nM, 50 nM to 200 nM, and more preferably in the range of 100 nM to 200 nM, but it is not limited to these ranges.

UvrD is a protein from E. coli with helicase activity that splits (unwinds) double-stranded DNA. Known UvrD, as well as those with mutations introduced into the amino acid sequence of known UvrD or with modified amino acids, can be used as long as they have helicase activity. In one embodiment, the wild-type UvrD from E. coli can be used. UvrD may be included in the range of 1 nM to 100 nM when acting on sequence errors, preferably in the range of 1 nM to 50 nM, 1 nM to 30 nM, 1 nM to 20 nM, 3 nM to 50 nM, 3 nM to 30 nM, 3 nM to 20 nM, 5 nM to 50 nM, 5 nM to 30 nM, 5 nM to 20 nM, 10 nM to 20 nM, and more preferably in the range of 15 nM, but it is not limited to these ranges.

Single-strand specific exonuclease is an enzyme that hydrolyzes nucleotides sequentially from the 3′ or 5′ end of linear single-stranded DNA. The single-strand specific exonuclease used in the present embodiment is not particularly restricted in type or biological origin as long as it has the enzyme activity to hydrolyze sequentially from the 3′ or 5′ end of linear DNA.

When using single-strand specific exonuclease together with UvrD, it can be appropriately selected and used according to the method of assembly/hybridization and the target DNA. For example, for double-stranded DNA obtained by assembling 5′ overhangs, it is preferable to use 5′-3′ exonuclease (5′→3′ exonuclease, an enzyme that degrades nucleotides from the 5′ end of DNA in the 3′ direction). For double-stranded DNA obtained by assembling 3′ overhangs, it is preferable to use 3′-5′ exonuclease (3′->5′ exonuclease, an enzyme that degrades nucleotides from the 3′ end of DNA in the 5′ direction).

When there are nicks or gaps in circular double-stranded DNA, UvrD can act on the DNA. On the other hand, MutH can act on circular double-stranded DNA without nicks or gaps, introducing nicks or leading to double-strand cleavage. In the case of circular double-stranded DNA with nicks or gaps, UvrD can invade from the nicks or gaps and unwind the single strand with helicase activity. In the case of linear double-stranded DNA, UvrD can invade from the ends and unwind the single strand even without nicks or gaps. In both circular and linear double-stranded DNA, if nicks or gaps are present on both strands of the double-stranded DNA at a distance, UvrD invading from one strand will unwind and split the single strand, leading to the separation of the double-stranded DNA when it reaches the nicks or gaps on the other strand. In this case, single-stranded overhang ends will be exposed after separation. Single-strand specific exonuclease can degrade and remove these single-stranded overhangs, preventing re-hybridization of the overhangs dissociated by UvrD. In circular double-stranded DNA, if nicks or gaps are present on both strands at a distance, the same fragmentation action of UvrD will lead to the linearization of the DNA.

When using single-strand specific exonuclease together with UvrD, examples of an enzyme that hydrolyzes sequentially from the 3′ end (3′→5′ single-strand specific exonuclease) include exonuclease VII, exonuclease I (Exo I), exonuclease T (Exo T) (also known as RNaseT), Exonuclease X, DNA Polymerase III Epsilon Subunit, DNA Polymerase I, DNA Polymerase II, T7 DNA Polymerase, T4 DNA Polymerase, Klenow DNA Polymerase, Phi29 DNA Polymerase, Ribonuclease III (RNase D), Oligoribonuclease (ORN), etc. Enzymes that hydrolyze sequentially from the 5′ end (5′ →3′ single-strand specific exonuclease) include Exonuclease VII, λ Exonuclease, Exonuclease VIII, T5 Exonuclease, T7 Exonuclease, and RecJ Exonuclease, etc.

ExoVII has both 5′-3′ and 3′-5′ single-strand specific exonuclease activities. Therefore, in one embodiment, ExoVII is preferred as a single-strand-specific exonuclease to be used with UvrD.

When single-strand-specific exonucleases are used together with MutS to act on double-stranded DNA during amplification reactions in cell-free systems, the single-strand-specific exonucleases are not limited as long as it can degrade the overhanging (protruding) ends of single-strands, and those described above can be used. In one embodiment the 3′→5′ single-strand-specific exonucleases and 5′→3′ single-strand-specific exonucleases listed above are preferred. For example, ExoVII, ExoI, ExoT, RecJ exonuclease, etc. can be used, with ExoI being particularly preferred.

The single-strand specific exonuclease may be used in an amount that exerts its activity according to the type used, and may be included in the range of 0.001 U/μL to 5 U/μL when acting on sequence errors, for example, 0.005 U/μL to 5 U/μL, preferably 0.01 U/μL to 3 U/μL but it is not limited to these ranges.

Reacting the mismatch repair-related enzyme group in contact with the double-stranded DNA mixture can conveniently be carried out in solution, for example, at 15-40° C., 16-40° C., 25-40° C., preferably 30-40° C., for 5-120 minutes, preferably 10-60 minutes. When a mismatch repair-related enzyme group is acted upon during the double-stranded DNA amplification reaction, it can be performed simultaneously under the reaction conditions of the amplification reaction. There is no restriction on the composition of the reaction solution as long as the action of the mismatch repair-related enzyme group can proceed. For example, the mismatch repair-related enzyme group can be added to a buffer solution such as Tris-HCl buffer, to which a magnesium ion source, ATP, etc. are added and additional components such as an alkali metal ion source are added as necessary. In particular, when the mismatch repair-related enzyme group contains UrvD, the reaction solution can contain ATP.

Buffers suitable for use at pH 7-9, preferably pH 8, can be used, such as Tris-HCl, Tris-OAc, Hepes-KOH, phosphate buffer, MOPS-NaOH, Tricine-HCl, etc. Preferred buffers are Tris-HCl or Tris-OAc. The concentration of the buffer solution can be selected by the person skilled in the art and is not limited; in the case of Tris-HCl or Tris-OAc, for example, concentrations of 10 mM to 100 mM, 10 mM to 50 mM, and 20 mM can be selected.

Magnesium ion sources are substances that provide magnesium ions (Mg²⁺) in the reaction solution. Examples include Mg(OAc)₂, MgCl₂, and MgSO₄, among others. A preferred magnesium ion source is Mg(OAc)₂. The concentration of the magnesium ion source in the reaction solution at the start of the reaction may be, for example, a concentration that gives magnesium ions in the range of 5-50 mM in the reaction solution.

ATP means adenosine triphosphate. The concentration of ATP in the reaction solution at the start of the reaction may be, for example, in the range of 0.1 mM to 3 mM, preferably 0.1 mM to 2 mM, 0.1 mM to 1.5 mM, or 0.5 mM to 1.5 mM.

An alkali metal ion source is a substance that provides alkali metal ions in the reaction solution. Examples of alkali metal ions are sodium ions (Na⁺) and potassium ions (K⁺). Examples of alkali metal ion sources include potassium glutamate, potassium aspartate, potassium chloride, potassium acetate, sodium glutamate, sodium aspartate, sodium chloride, and sodium acetate. Preferred alkali metal ion sources are potassium glutamate or potassium acetate. The concentration of the alkali metal ion source in the reaction solution at the start of the reaction may be, but is not limited to, a concentration that provides alkali metal ions in the reaction solution in the range of 100 mM or more, preferably 100 mM to 300 mM.

The reaction solution in which the mismatch repair-related enzyme group is added to the double-stranded DNA mixture (it may be the amplification reaction solution containing the double-stranded DNA mixture in which the mismatch repair-related enzyme group is added) is submitted to the double-stranded DNA amplification reaction either as is or after allowing the mismatch repair-related enzyme group to act on the double-stranded DNA molecule having sequence error in said reaction solution. The amplification method is not limited, and may be cell-free amplification or intracellular amplification. In the case of cell-free amplification, it may be non-isothermal, as represented by PCR, or isothermal amplification. Among them, isothermal amplification or amplification performed at a temperature of 80° C. or lower is preferred, and amplification performed at a temperature of 65° C. or lower is more preferred. For example, incubation is performed at a certain temperature in the range of 15° C. to 80° C., 16° C. to 80° C., 20° C. to 80° C., 15° C. to 75° C., 16° C. to 75° C., 20° C. to 75° C., 15° C. to 70° C., 16° C. to 70° C., 20° C. to 70° C., preferably in the range of 16° C. to 65° C. Alternatively, amplification is performed under a temperature cycle of repeated incubations at two temperatures 80° C. or lower, 75° C. or lower, 70° C. or lower, and preferably 65° C. or lower.

The double-stranded DNA to be amplified can be selected according to the purpose of use, and may be linear or circular, with circular being preferred in one embodiment. The size of the amplification target is not particularly limited as long as it can be amplified by the chosen amplification method. For example, it can be 1 kb (1000 bases in length) or longer, 2 kb or longer, 3 kb or longer, 5 kb or longer, 8 kb or longer, 10 kb or longer, 50 kb or longer, 100 kb or longer, and 1 Mb (1 million bases in length) or shorter, 100 kb or shorter, 50 kb or shorter, 30 kb or shorter, 20 kb or shorter, 10 kb or shorter. In one embodiment, it can be preferably 1 kb or longer and 50 kb or shorter, more preferably 1 kb or longer and 30 kb or shorter, e.g.,2 kb or longer and 20 kb or shorter, or 3 kb or longer and 10 kb or shorter.

For amplification in cells, techniques known in the technical field can be used. For example, the type of cells, such as E. coli, Bacillus subtilis, yeast, etc., is not particularly limited. Double-stranded DNA can be prepared as appropriate for the cell to be used. For simplicity, circular double-stranded DNA with a replication origin can be prepared and introduced into E. coli for amplification.

Amplification in a cell-free system can also use techniques known in the art. When using isothermal amplification in a cell-free system, various techniques are known (e.g., J. Li and J. Macdonald, Biosensors and Bioelectronics, 2015, vol. 64, p. 196-211), including, but not limited to, Helicase-dependent amplification (HDA) (Vincent, et al, EMBO Rep., 2004, vol. 5 (8), p. 795-800), Recombinase polymerase amplification (RPA) (Piepenburg, et al, PLOS. Biol., 2006, vol. 4 (7), e204), rolling circle amplification (RCA) (Fire, et al, Proc. Natl. Acad. Sci., 1995, vol. 92 (10), p. 4641-4645), Ramification amplification (RAM) (Zhang, et al, Mol. Diagn., 2001, vol. 6 (2), p.141-150), Multiple displacement amplification (MDA) (Dean, et al, Genome Res. Genome Res., 2001, vol. 11 (6), p.1095-1099, and Spits, et al, Nat. Protoc., 2006, vol. 1 (4), p.1965-1970), loop-mediated isothermal amplification (LAMP) (Notomi, et al, Nucleic Acids Res., 2000, vol. 28 (12), E63), and other known techniques can be used. When non-isothermal amplification in a cell-free system is used, known techniques such as PCR can be used. Either method can be performed according to the established method, and the choice of which method to use can be made according to the shape of the DNA to be amplified (linear or circular) and other factors.

In one embodiment, DNA can be amplified using the Replication Cycle Reaction method (hereinafter referred to as the RCR method; see WO2016/080424, WO2017/199991, WO2018/159669) The RCR method is a method for amplifying circular DNA comprising the following steps, and the circular DNA amplified by the RCR method contains a replication origin sequence (e.g., oriC) that can bind to an enzyme having DnaA activity:
(a) forming a reaction mixture of circular DNA to be amplified with a reaction solution comprising:
a first enzyme group that catalyzes replication of circular DNA,
a second enzyme group that catalyzes an Okazaki fragment maturation and synthesizes two sister circular DNAs constituting a catenane, and
a third enzyme group that catalyzes a separation of two sister circular DNAs; and
(b) incubating the reaction mixture prepared in step (a) at a constant temperature within the range of 15° C. to 80° C. or under a temperature cycle of repeated incubations at two temperatures below 80° C.

As the first enzyme group that catalyzes the replication of circular DNA, for example, the enzyme group described in Kaguni J M & Kornberg A. Cell. 1984, 38:183-90 can be used. Specifically, examples of the first enzyme group include one or more enzymes or enzyme group selected from a group consisting of an enzyme having DnaA activity, one or more types of nucleoid protein, an enzyme or enzyme group having DNA gyrase activity, single-strand binding protein (SSB), an enzyme having DnaB-type helicase activity, an enzyme having DNA helicase loader activity, an enzyme having DNA primase activity, an enzyme having DNA clamp activity, and an enzyme or enzyme group having DNA polymerase III* activity, and a combinations of all of the aforementioned enzymes or enzyme groups. In one embodiment, the first enzyme group includes an enzyme having DnaA activity, single-strand binding protein (SSB), an enzyme having DnaB-type helicase activity, an enzyme having DNA helicase loader activity, an enzyme having DNA primase activity, an enzyme having DNA clamp activity, and an enzyme or enzyme group having DNA polymerase III* activity.

The enzyme having DnaA activity is not particularly limited in its biological origin as long as it has an initiator activity that is similar to that of DnaA, which is an initiator protein of E. coli, and DnaA derived from E. coli may be preferably used. The Escherichia coli-derived DnaA may be contained as a monomer in the reaction solution in an amount of 1 nM to 10 μM, preferably in an amount of 1 nM to 5 μM, 1 nM to 3 μM, 1 nM to 1.5 μM, 1 nM to 1.0 μM, 1 nM to 500 nM, 50 nM to 200 nM, or 50 nM to 150 nM, but without being limited thereby.

A nucleoid protein is protein in the nucleoid. The one or more type of nucleoid protein used in the present invention is not particularly limited in its biological origin as long as it has an activity that is similar to that of the nucleoid protein of E. coli. For example, Escherichia coli-derived IHF, namely, a complex of IhfA and/or IhfB (a heterodimer or a homodimer), or Escherichia coli-derived HU, namely, a complex of hupA and hupB can be preferably used. The Escherichia coli-derived IHF may be contained as a hetero/homo dimer in a reaction solution in a concentration range of 5 nM to 400 nM. Preferably, the Escherichia coli-derived IHF may be contained in a reaction solution in a concentration range of 5 nM to 200 nM, 5 nM to 100 nM, 5 nM to 50 nM, 10 nM to 50 nM, 10 nM to 40 nM, or 10 nM to 30 nM, but the concentration range is not limited thereto. The Escherichia coli-derived HU may be contained in a reaction solution in a concentration range of 1 nM to 50 nM, and preferably, may be contained therein in a concentration range of 5 nM to 50 nM or 5 nM to 25 nM, but the concentration range is not limited thereto.

An enzyme or enzyme group having DNA gyrase activity is not particularly limited in its biological origin as long as it has an activity that is similar to that of the DNA gyrase of E. coli. For example, a complex of Escherichia coli-derived GyrA and GyrB can be preferably used. Such a complex of Escherichia coli-derived GyrA and GyrB may be contained as a heterotetramer in a reaction solution in a concentration range of 20 nM to 500 nM, and preferably, may be contained therein in a concentration range of 20 nM to 400 nM, 20 nM to 300 nM, 20 nM to 200 nM, 50 nM to 200 nM, or 100 nM to 200 nM, but the concentration range is not limited thereto.

A single-strand binding protein (SSB) is not particularly limited in its biological origin as long as it has an activity that is similar to that of the single-strand binding protein of E. coli. For example, Escherichia coli-derived SSB can be preferably used. Such Escherichia coli-derived SSB may be contained as a homotetramer in a reaction solution in a concentration range of 20 nM to 1000 nM, and preferably, may be contained therein in a concentration range of 20 nM to 500 nM, 20 nM to 300 nM, 20 nM to 200 nM, 50 nM to 500 nM, 50 nM to 400 nM, 50 nM to 300 nM, 50 nM to 200 nM, 50 nM to 150 nM, 100 nM to 500 nM, or 100 nM to 400 nM, but the concentration range is not limited thereto.

An enzyme having DnaB-type helicase activity is not particularly limited in its biological origin as long as it has an activity that is similar to that of the DnaB of E. coli. For example, Escherichia coli-derived DnaB can be preferably used. Such Escherichia coli-derived DnaB may be contained as a homohexamer in a reaction solution in a concentration range of 5 nM to 200 nM, and preferably, may be contained therein in a concentration range of 5 nM to 100 nM, 5 nM to 50 nM, or 5 nM to 30 nM, but the concentration range is not limited thereto.

An enzyme having DNA helicase loader activity is not particularly limited in its biological origin as long as it has an activity that is similar to that of the DnaC of E. coli. For example, Escherichia coli-derived DnaC can be preferably used. Such Escherichia coli-derived DnaC may be contained as a homohexamer in a reaction solution in a concentration range of 5 nM to 200 nM, and preferably, may be contained therein in a concentration range of 5 nM to 100 nM, 5 nM to 50 nM, or 5 nM to 30 nM, but the concentration range is not limited thereto.

An enzyme having DNA primase activity is not particularly limited in its biological origin as long as it has an activity that is similar to that of the DnaG of E. coli. For example, Escherichia coli-derived DnaG can be preferably used. Such Escherichia coli-derived DnaG may be contained as a monomer in a reaction solution in a concentration range of 20 nM to 1000 nM, and preferably, may be contained therein in a concentration range of 20 nM to 800 nM, 50 nM to 800 nM, 100 nM to 800 nM, 200 nM to 800 nM, 250 nM to 800 nM, 250 nM to 500 nM, or 300 nM to 500 nM, but the concentration range is not limited thereto.

An enzyme having DNA clamp activity is not particularly limited in its biological origin as long as it has an activity that is similar to that of the DnaN of E. coli. For example, Escherichia coli-derived DnaN can be preferably used. Such Escherichia coli-derived DnaN may be contained as a homodimer in a reaction solution in a concentration range of 10 nM to 1000 nM, and preferably, may be contained therein in a concentration range of 10 nM to 800 nM, 10 nM to 500 nM, 20 nM to 500 nM, 20 nM to 200 nM, 30 nM to 200 nM, or 30 nM to 100 nM, but the concentration range is not limited thereto.

An enzyme or enzyme group having DNA polymerase III* activity is not particularly limited in its biological origin as long as it is an enzyme or enzyme group having an activity that is similar to that of the DNA polymerase III* complex of E. coli. For example, an enzyme group comprising any of Escherichia coli-derived DnaX, HolA, HolB, HolC, HolD, DnaE, DnaQ, and HolE, preferably, an enzyme group comprising a complex of Escherichia coli-derived DnaX, HolA, HolB, and DnaE, and more preferably, an enzyme comprising a complex of Escherichia coli-derived DnaX, HolA, HolB, HolC, HolD, DnaE, DnaQ, and HolE, can be preferably used. Such an Escherichia coli-derived DNA polymerase III* complex may be contained as a heteromultimer in a reaction solution in a concentration range of 2 nM to 50 nM, and preferably, may be contained therein in a concentration range of 2 nM to 40 nM, 2 nM to 30 nM, 2 nM to 20 nM, 5 nM to 40 nM, 5 nM to 30 nM, or 5 nM to 20 nM, but the concentration range is not limited thereto.

In the present invention, two sister circular DNAs constituting a catenane are two circular DNAs synthesized by DNA replication, then joined together.

Examples of second enzyme groups that catalyze an Okazaki fragment maturation and synthesize two sister circular DNAs constituting a catenane may be, for example, one or more enzymes selected from the group consisting of an enzyme having DNA polymerase I activity, an enzyme having DNA ligase activity, and an enzyme having RNaseH activity, or a combination of these enzymes. In one embodiment, the second enzyme group preferably includes an enzyme having DNA polymerase I activity and an enzyme having DNA ligase activity.

An enzyme having DNA polymerase I activity is not particularly limited in its biological origin as long as it has an activity that is similar to DNA polymerase I of E. coli. For example, Escherichia coli-derived DNA polymerase I can be preferably used. Such Escherichia coli-derived DNA polymerase I may be contained as a monomer in a reaction solution in a concentration range of 10 nM to 200 nM, and preferably, may be contained therein in a concentration range of 20 nM to 200 nM, 20 nM to 150 nM, 20 nM to 100 nM, 40 nM to 150 nM, 40 nM to 100 nM, or 40 nM to 80 nM, but the concentration range is not limited thereto.

An enzyme having DNA ligase activity is not particularly limited in its biological origin as long as it has an activity that is similar to DNA ligase of E. coli. For example, Escherichia coli-derived DNA ligase or the DNA ligase of T4 phage can be preferably used. Such Escherichia coli-derived DNA ligase may be contained as a monomer in a reaction solution in a concentration range of 10 nM to 200 nM, and preferably, may be contained therein in a concentration range of 15 nM to 200 nM, 20 nM to 200 nM, 20 nM to 150 nM, 20 nM to 100 nM, or 20 nM to 80 nM, but the concentration range is not limited thereto.

The enzyme having RNaseH activity is not particularly limited in terms of biological origin, as long as it has the activity of decomposing the RNA chain of an RNA-DNA hybrid. For example, Escherichia coli-derived RNaseH can be preferably used. Such Escherichia coli-derived RNaseH may be contained as a monomer in a reaction solution in a concentration range of 0.2 nM to 200 nM, and preferably, may be contained therein in a concentration range of 0.2 nM to 200 nM, 0.2 nM to 100 nM, 0.2 nM to 50 nM, 1 nM to 200 nM, 1 nM to 100 nM, 1 nM to 50 nM, or 10 nM to 50 nM, but the concentration range is not limited thereto.

An example of a third enzyme group that catalyzes a separation of two sister circular DNAs is an enzyme group set forth in, for example, the enzyme group described in Peng H & Marians K J. PNAS. 1993, 90:8571-8575. Specifically, examples of the third enzyme group include one or more enzymes selected from a group consisting of an enzyme having topoisomerase IV activity, an enzyme having topoisomerase III activity, and an enzyme having RecQ-type helicase activity; or a combination of the aforementioned enzymes. In one embodiment, the third enzyme group includes an enzyme having topoisomerase IV activity and/or an enzyme having topoisomerase III activity,

The enzyme having topoisomerase III activity is not particularly limited in terms of biological origin, as long as it has the same activity as that of the topoisomerase III of Escherichia coli. For example, Escherichia coli-derived topoisomerase III can be preferably used. Such Escherichia coli-derived topoisomerase III may be contained as a monomer in a reaction solution in a concentration range of 20 nM to 500 nM, and preferably, may be contained therein in a concentration range of 20 nM to 400 nM, 20 nM to 300 nM, 20 nM to 200 nM, 20 nM to 100 nM, or 30 to 80 nM, but the concentration range is not limited thereto.

The enzyme having RecQ-type helicase activity is not particularly limited in terms of biological origin, as long as it has the same activity as that of the RecQ of Escherichia coli. For example, Escherichia coli-derived RecQ can be preferably used. Such Escherichia coli-derived RecQ may be contained as a monomer in a reaction solution in a concentration range of 20 nM to 500 nM, and preferably, may be contained therein in a concentration range of 20 nM to 400 nM, 20 nM to 300 nM, 20 nM to 200 nM, 20 nM to 100 nM, or 30 to 80 nM, but the concentration range is not limited thereto.

An enzyme having topoisomerase IV activity is not particularly limited in its biological origin as long as it has an activity that is similar to topoisomerase IV of E. coli. For example, Escherichia coli-derived topoisomerase IV that is a complex of ParC and ParE can be preferably used. Such Escherichia coli-derived topoisomerase IV may be contained as a heterotetramer in a reaction solution in a concentration range of 0.1 nM to 50 nMM, and preferably, may be contained therein in a concentration range of 0.1 nM to 40 nM, 0.1 nM to 30 nM, 0.1 nM to 20 nM, 1 nM to 40 nM, 1 nM to 30 nM, 1 nM to 20 nM, 1 nM to 10 nM, or 1 nM to 5 nM, but the concentration range is not limited thereto.

The reaction solution may contain further enzymes. For example, if the circular DNA to be amplified by the RCR method has a pair of ter sequences, each inserted outward relative to a replication origin sequence (e.g., oriC) that can bind to an enzyme having DnaA activity, the reaction solution may also contain a protein (e.g., Tus protein from E. coli) that binds to the ter sequence and has replication inhibitory activity.

The first, second and third enzyme groups given above may be those that are commercially available, or they may be extracted from microorganisms and purified as necessary. Extraction and purification of enzymes from microorganisms may be performed as necessary using means that are available to a person skilled in the art.

When enzymes other than the above-described Escherichia coli-derived enzymes are used as the first, second and third enzyme groups, they may be each used in a concentration range corresponding, as an enzyme activity unit, to the concentration range that is specified with respect to the above-described Escherichia coli-derived enzyme.

The reaction solution may include buffer, ATP, GTP, CTP, UTP, dNTP, magnesium ion source, and alkali metal ion source. The buffer solution, magnesium ion source, ATP, and alkali metal ion source can be the same as those described above for the reaction solution for the action of mismatch repair-related enzyme group. The reaction solution can also contain protein non-specific adsorption inhibitor (bovine serum albumin, lysozyme, gelatin, heparin, casein, etc.), nucleic acid non-specific adsorption inhibitor (tRNA (transfer RNA), rRNA (ribosomal RNA), mRNA (messenger RNA), glycogen, heparin, Oligo DNA, poly (I-C) (polyinosine-polycytidine), poly(dI-dC) (poly deoxyinosine-polydeoxycytidine), poly(A) (polyadenine) and poly(dA) (polydeoxyadenine)), linear DNA-specific exonucleases (RecBCD, lambda exonuclease, exonuclease III, exonuclease VIII, T5 exonuclease, T7 exonuclease, Plasmid Safe (registered trademark) ATP-Dependent DNase (epicentre), etc.), RecG-type helicase (RecG from E. coli, etc.), ammonium salts (ammonium sulfate, ammonium chloride, ammonium acetate, etc.), reducing agents (DTT, β-mercaptoethanol, glutathione, etc.), and if the reaction solution contains DNA ligase from E. coli, its cofactor, NAD (nicotinamide adenine dinucleotide), may be included.

The reaction solution containing the above enzyme and the cell-free protein expression system may be mixed as it is with circular DNA as a template to form a reaction mixture for amplification of circular DNA. The cell-free protein expression system may be a cell-free translation system that comprises a total RNA containing RNA consisting of a sequence that is complementary to the base sequence of genes encoding the above enzymes, mRNA or in vitro transcription product as the template RNA, or it may be a cell-free transcription/translation system that comprises genes encoding different enzymes or expression vectors including genes that encode different enzymes as the template DNA.

When double-stranded DNA is amplified by the RCR method, the double-stranded DNA is circular DNA and has a replication origin sequence that can bind to an enzyme having DnaA activity. The replication origin sequence that can bind to an enzyme having DnaA activity can be obtained from public databases such as NCBI. The replication origin sequence can also be obtained by cloning a DNA fragment that can bind to the enzyme having DnaA activity and analyzing the sequence. A modified replication origin sequence in which one or more bases of a known replication origin sequence are substituted, deleted, or inserted, and capable of binding to the enzyme having DnaA activity can also be used. The replication origin sequence used in the present embodiment is preferably oriC and modified sequences thereof, and more preferably oriC from E. coli and modified sequences thereof.

In the case of isothermal amplification of double-stranded DNA, there are no restrictions as to the isothermal conditions as far as the DNA amplification reaction or DNA replication reaction can proceed. For example, the temperature can be set to a certain temperature within the optimum temperature of DNA polymerase. Isothermal conditions include, for example, a constant temperature of 15° C. or higher, 16° C. or higher, 20° C. or higher, 25° C. or higher, or 30° C. or higher, and a constant temperature of 80° C. or lower, 75° C. or lower, 70° C. or lower, 65° C. or lower, 60° C. or lower, 50° C. or lower, 45° C. or lower, 40° C. or lower, 35° C. or lower, or 33° C. or lower. Isothermal conditions are, for example, a constant temperature included in the range of 15° C. to 80° C., 16° C. to 80° C., or 20° C. to 80° C.; a constant temperature included in the range of 15° C. to 75° C., 16° C. to 75° C., or 20° C. to 75° C.; a constant temperature included in the range of 15° C. to 70° C., 16° C. to 70° C., or 20° C. to 70° C., a constant temperature included in the range of 15° C. to 65° C., 16° C. to 65° C., or 20° C. to 65° C., a constant temperature included in the range of 25° C. to 50° C., a constant temperature included in the range of 25° C. to 40° C., a constant temperature included in the range of 30° C. to 33° C., or about 30° C. In this specification, “isothermal” in isothermal amplification means to keep the temperature within +7° C., +5° C., +3° C., or +1° C. relative to the temperature set during the reaction. The reaction time for isothermal amplification can be set according to the amount of the target double-stranded DNA amplification product, for example, 1 to 30 hours, preferably 6 to 24 hours, more preferably 16 to 24 hours, and more preferably 18 to 21 hours.

When double-stranded DNA is amplified under temperature conditions where the double-stranded DNA is incubated under a temperature cycle of repeated incubations at two temperatures below 80° C., preferably below 65° C., the double-stranded DNA is preferably circular DNA. The first temperature of the temperature cycle is the temperature at which replication initiation of the double-stranded DNA is possible, and the second temperature is the temperature at which replication initiation is inhibited and the DNA elongation reaction proceeds. The first temperature can be 30° C. or higher, e.g., 30° C. to 80° C., 30° C. to 50° C., 30° C. to 40° C., or 37° C. Incubation at the first temperature is not limited, but may be 10 seconds to 10 minutes per cycle, with 1 minute being preferred. The second temperature can be 27° C. or lower, e.g., 10° C. to 27° C., 16° C. to 25° C., or 24° C. Incubation at the second temperature is not particularly limited, but preferably be set according to the length of the circular DNA to be amplified, for example, it may be 1 to 10 seconds per 1000 bases per cycle. The number of temperature cycles is not particularly limited, but may be 10 to 50 cycles, 20 to 45 cycles, 25 to 45 cycles, or 40 cycles.

In the method of the present embodiment, double-stranded DNA molecules with sequence error are not amplified and double-stranded DNA molecules without sequence error are amplified, so that the ratio of double-stranded DNA molecules with sequence errors to amplified double-stranded DNA is reduced compared to the ratio of double-stranded DNA molecules with sequence errors to double-stranded DNA before performing the method of the present embodiment, or compared to the ratio of double-stranded DNA molecules with sequence errors relative to double-stranded DNA amplified without the addition of the mismatch repair-related enzyme group. In one embodiment, for example, it is reduced to 50%, 25%, 10% or less. In this connection, compared to the double-stranded DNA before amplification, the double-stranded DNA is amplified, for example, more than 10-fold, 50-fold, 100-fold, 200-fold, 500-fold, 1000-fold, 2000-fold, 3000-fold, 4000-fold, 5000-fold, or 10000-fold.

The confirmation of whether the percentage of double-stranded DNA molecules with sequence errors has decreased can be performed by conventional methods. For example, NGS analysis, as shown in the examples below, can be used. It is also possible to confirm the percentage of sequence errors by measuring the number of colonies, which can be designed so that the colonies of E. coli transformed with double-stranded DNA can be color-coded according to the presence or absence of sequence errors. As shown in the example below, if the double-stranded DNA is designed to be cleaved with the desired cleavage enzyme only when it does not have sequence errors, the size of the DNA fragments obtained using restriction enzymes can be checked by size separation methods such as gel electrophoresis to confirm that the percentage of double-stranded DNA molecules with sequence errors is reduced.

The method of the present embodiment includes amplifying double-stranded DNA and allowing a mismatch repair-related enzyme group to act on a double-stranded DNA molecule having a sequence error, wherein the proportion of double-stranded DNA with sequence errors is reduced whether (a) the mismatch repair-related enzyme group is allowed to act prior to amplifying the double-stranded DNA or (b) the mismatch repair-related enzyme group is applied simultaneously with the amplification of the double-stranded DNA. Only one of (a) and (b) may be performed, or both may be performed together.

In one embodiment, in the method of the present embodiment, when a mismatch repair-related enzyme group is reacted on a double-stranded DNA molecule having a sequence error prior to amplification of the double-stranded DNA, it is preferred that the mismatch repair-related enzyme group, in addition to MutS and MutL, further includes an enzyme selected from MutH, UvrD, and a combination of UvrD and a single-strand-specific exonuclease. Among the enzymes included in addition to MutS and MutL, it is more preferred to include at least UvrD, it is even more preferred to include UvrD and a single-strand specific exonuclease, and it is especially preferred to include UvrD and ExoVII.

Although not bound by theory, the addition of MutL in addition to MutS forms a MutS-MutL complex at the site of the sequence error that is more stable than MutS alone. This complex inhibits the action of a group of DNA replication enzymes in the subsequent DNA amplification process, which is thought to inhibit the amplification of double-stranded DNA molecules with sequence error.

Although not bound by theory, when UvrD is added in addition to MutS and MutL, UvrD that enters through a nick or gap near a sequence error via the MutS-MutL complex will unwind the single strand. If the other strand also has a nick or gap, this leads to the fragmentation of linear DNA or the linearization of circular DNA. This is thought to inhibit the amplification of double-stranded DNA with sequence error, as DNA synthase will drop out at the fragmented portion if the template DNA is fragmented in the middle during DNA amplification. Further, in DNA amplification reactions that require circular DNA as template DNA, the amplification of double-stranded DNA molecules with sequence errors is considered to be inhibited by the fact that the double-stranded DNA molecules with sequence errors become linear instead of circular. Furthermore, in intracellular DNA amplification (cloning) using E. coli as a host, linearized DNA is quickly degraded and eliminated in the cell, so that amplification of double-stranded DNA with sequence error does not occur and only double-stranded DNA molecules without sequence error and retaining their circular form are considered to be amplified.

The fragmented surface of the DNA fragmented by the action of UvrD exposes the overhanging (protruding) end of the single-stranded DNA. The single-strand-specific exonuclease degrades and removes the overhanging portion of the single-strand and inhibits the reconnection of the fragmented portion, thus suppressing the return of double-stranded DNA with sequence errors to a state where DNA can be amplified again.

Although not bound by theory, when MutH is added in addition to MutS and MutL, the endonuclease activity of MutH acts near the sequence error via the MutS-MutL complex, leading to double-stranded DNA cleaves. In the case of circular DNA, linearization of DNA is induced. The mechanism by which the amplification of cleaved or linearized double-stranded DNA is inhibited is as described in the description of UvrD. In one embodiment, when the double-stranded DNA is circular, it is preferred that the mismatch repair-related enzyme group further include MutH in addition to MutS and MutL.

When a mismatch repair-related enzyme group is to act on a double-stranded DNA molecule having a sequence error prior to amplification of the double-stranded DNA, it is preferred that the double-stranded DNA has a nick or gap or is linearly stranded in one embodiment. Among others, a DNA obtained by hybridizing a part or all of, preferably a part of: a combination of single-stranded DNA with single-stranded DNA that is complementary to said single-stranded DNA; a combination of double-stranded DNA having a single-stranded portion and single-stranded DNA having a base sequence complementary to at least one portion of said single-stranded portion; or a combination of double-stranded DNA having a single-stranded portion and double-stranded DNA with single-stranded portion with a sequence complementary to at least a part of said single-stranded portion, is preferred. In one embodiment, when the method of amplification of double-stranded DNA is a method of amplification of circular double-stranded DNA, the double-stranded DNA is preferably circular double-stranded DNA.

In one embodiment, prior to amplification of the double-stranded DNA, a mismatch repair-related enzyme group is allowed to act on the double-stranded DNA molecule with the sequence error, and if the mismatch repair-related enzyme group includes MutS, MutL, UvrD and a single-strand specific exonuclease, subsequent amplification of the double-stranded DNA may be performed intracellularly.

In one embodiment, in the method of the present embodiment, when a mismatch repair-related enzyme group is reacted on a double-stranded DNA molecule having a sequence error in the same reaction solution at the same time as the amplification of the double-stranded DNA, the mismatch repair-related enzyme group preferably includes, in addition to MutS and MutL, also MutH and/or a single strand-specific exonuclease (e.g., ExoI), or the mismatch repair-related enzyme group preferably includes, in addition to MutS and a single-strand-specific exonuclease (e.g., ExoI), MutL and/or MutH, and particularly preferably, the mismatch repair-related enzyme group includes MutS, MutL, MutH and single strand-specific exonuclease (e.g., ExoI). In this case, it is also preferred to amplify double-stranded DNA by a method that can amplify circular DNA in a cell-free system (preferably the RCR method).

Although not bound by theory, the addition of MutL in addition to MutS (or MutS and single-strand specific exonuclease) forms a MutS-MutL complex that is more stable than MutS alone at the site of sequence errors. This complex inhibits the action of a group of DNA replication enzymes in the DNA amplification process, which is thought to inhibit the amplification of double-stranded DNA molecules with sequence errors.

Although not bound by theory, the addition of a single-strand specific exonuclease in addition to MutS (or MutS and MutL) may inhibit assembly and cyclization during amplification of double-stranded DNA molecules with sequence errors because the degradation of the overhang regions of the sequence error sites (e.g., incomplete DNA products where oligo DNA assembly did not proceed) and/or the degradation of overhang regions derived from double-stranded DNA cleavage caused by the inhibition of replication at the site of action of MutS occurs, and inhibits the assembly and cyclization during amplification of double-stranded DNA molecules with sequence errors. In addition, although not bound by theory, if MutH is present in addition to MutS (or MutS and MutL) and a single-strand-specific exonuclease, the endonuclease activity of MutH in the vicinity of the sequence error may act to cleave the double-stranded DNA. Then, the single-strand-specific exonuclease further react on the cleaved site. This is thought to prevent re-cyclization of the cleavage product.

In one case, a mismatch repair-related enzyme group may react on the double-stranded DNA with the sequence error prior to amplification of the double-stranded DNA, and furthermore, the mismatch repair-related enzyme group may be allowed to act on the double-stranded DNA with the sequence error during amplification of the double-stranded DNA. In this case, the mismatch repair-related enzyme group used prior to amplification may be allowed to act during amplification of the double-stranded DNA without inactivating their action. For example, the reaction solution in which the mismatch repair-related enzyme group were acted upon prior to amplification of the double-stranded DNA may be added directly to the reaction solution for amplification of the double-stranded DNA, and upon doing so, one or more enzymes from the mismatch repair-related enzyme group may be further added.

The present embodiment also relates to a method for producing double-stranded DNA using a double-stranded DNA amplification reaction, comprising:
subjecting a reaction solution containing mismatch repair-related enzyme group and double-stranded DNA to the double-stranded DNA amplification reaction, wherein
the mismatch repair-related enzyme group comprises: MutS and MutL; or MutS and single strand-specific exonuclease, and
   the double-stranded DNA amplification reaction is a cell-free amplification reaction. Preferably, said amplification reaction is performed at a temperature of 65° C. or lower.

The double-stranded DNA amplification reaction in this method is not limited to any amplification reaction in which double-stranded DNA with sequence errors can occur during amplification of double-stranded DNA. Since the efficient removal of double-stranded DNA molecules with sequence errors in the present invention is fully demonstrated, the double-stranded DNA amplification reaction in which double-stranded DNA with sequence errors selected from base substitution, base insertion and base deletion on one strand of the double strands (simply referred to as “amplification error”) is generated is preferred. Specifically, any of the amplification reactions described above may be used. Among them, amplification reactions in a cell-free system performed at a temperature of 65° C. or lower are preferred.

The double-stranded DNA used in the amplification reaction is also not limited and can be selected according to the purpose of use after amplification and the amplification reaction. In one embodiment, the double-stranded DNA is preferably circular, contains a nick or gap, or both. The method of providing the double-stranded DNA for the amplification reaction is also not particularly limited, and may be, for example, assembly of fragments of double-stranded DNA. In one embodiment, for example, double-stranded DNA obtained by hybridizing a part or all of the single-stranded portion of one or more combination selected from:
a combination of single-stranded DNA and a single-stranded DNA that is the complementary strand of said single-stranded DNA,
a combination of double-stranded DNA having a single-stranded portion and a single-stranded DNA having a base sequence complementary to at least one portion of said single-stranded portion, and
a combination of double-stranded DNA with a single-stranded portion and a double-stranded DNA with a single-stranded portion having a base sequence complementary to at least one portion of said single-stranded portion. The double-stranded DNA thus obtained may contain nicks or gaps.

The percentage of double-stranded DNA with sequence errors selected from base substitutions, base insertions, and base deletions in one of the double strands to the double-stranded DNA amplified by this method described above is reduced compared to the percentage of double-stranded DNA with the above sequence errors to the double-stranded DNA amplified under the same conditions except that the reaction solution does not contain a mismatch repair-related enzyme group.

In one embodiment, the method of hybridizing all or part of the single-stranded portion of: a single-stranded DNA and a single-stranded DNA that is the complementary strand of said single-stranded DNA; a double-stranded DNA having a single-stranded portion and a single-stranded DNA having a base sequence complementary to at least one portion of said single-stranded portion; or a double-stranded DNA with a single-stranded portion and a double-stranded DNA with a single-stranded portion having a base sequence complementary to at least one portion of said single-stranded portion, is not particularly limited. Hybridization may be done by annealing under stringent conditions or by assembling a part or all of the single strands together using enzymes. It may also be hybridization of multiple single-stranded DNAs and their complementary strands at once. Techniques for assembly of single-stranded DNA in cell-free systems include In fusion method, Gibson Assembly method, Golden Gate Assembly method, Recombination Assembly method (RA method; WO2019/009361), USER (registered trademark) Cloning (NEB) and other methods using commercially available kits, and such known techniques can be used.

When using the RA method, a reaction solution containing two or more types of DNA fragments and a protein with RecA family recombinase activity is prepared. When at least one of the DNA fragments to be assembled is a linear double-stranded DNA fragment, said reaction solution further contains an exonuclease. Then, in said reaction solution, said two or more types of DNA fragments are assembled together in regions of complementary base sequences to obtain linear or circular DNA. For each component used in the RA method, see WO 2019/009361. For example, as an exonuclease, as long as it has enzymatic activity to hydrolyze DNA sequentially from the 3′ or 5′ end, there is no restriction on the type or biological origin of the exonuclease. For example, linear double-stranded DNA-specific 3′→5′ exonucleases such as AP (apurinic/apyrimidinic) endonucleases of the exonuclease III family can be preferably used. The exonuclease to be used is preferably both a linear double-stranded DNA-specific 3′→5′ exonuclease and a single-stranded DNA-specific 3′→5′ exonuclease, such as, AP endonucleases of the exonuclease III family type in combination with one or more single-stranded DNA-specific 3′→5′ exonucleases (such as DnaQ superfamily proteins) can be used. Specifically, for example, a combination of exonuclease III and exonuclease I, or a combination of exonuclease III, exonuclease I and exonuclease T can be used.

The RecA family recombinase proteins used in the RA method are any proteins that polymerize to form filaments on single-stranded or double-stranded DNA, have hydrolytic activity against nucleoside triphosphates such as ATP (adenosine triphosphate), and have the ability to search homologous regions and perform homologous recombination (RecA family recombinase activity). Prokaryotic RecA homologs such as E. coli RecA, bacteriophage RecA homologs such as T4 phage UvsX, archaeal RecA homologs, eukaryotic RecA homologs, etc., as well as variants thereof which retain RecA family recombinase activity, can be used.

Nucleoside triphosphate or deoxynucleotide triphosphate is required for RecA family recombinase proteins to exhibit RecA family recombinase activity. Therefore, the reaction solution for the RA method contains at least one of nucleoside triphosphate and deoxynucleotide triphosphate. The nucleoside triphosphates to be included in the reaction solution for the assembly reaction in the RA method are preferably one or more selected from a group consisting of ATP, GTP (guanosine triphosphate), CTP (cytidine triphosphate), UTP (uridine triphosphate) and m5 UTP (5-methyluridine triphosphate) and ATP is particularly preferred. The deoxynucleoside triphosphate to be included in the reaction solution in the RA method are preferably one or more selected from a group consisting of dATP (deoxyadenosine triphosphate), dGTP (deoxyguanosine triphosphate), dCTP (deoxycytidine triphosphate), and dTTP (deoxythymidine triphosphate), and dATP is particularly preferred. The total amount of nucleoside triphosphate and deoxynucleotide triphosphate in the reaction solution is not limited as long as it is sufficient for the RecA family recombinase protein to exhibit RecA family recombinase activity. As the nucleoside triphosphate concentration or deoxynucleotide triphosphate concentration in the reaction solution for the assembly reaction by RA method, for example, 1 μM (μmol/L) or more is preferred, 10 μM or more is more preferred, 30 UM or more is even more preferred, and 100 μM or more is especially preferred at the start of the assembly reaction to the total volume of the reaction solution. On the other hand, in order not to decrease the assembly efficiency, 1000 μM or less is preferred, 500 μM or less is more preferred, and 300 μM or less is even more preferred as the nucleoside triphosphate or deoxynucleotide triphosphate concentration in the reaction solution at the start of the assembly reaction with respect to the total volume of the reaction solution.

Magnesium ions (Mg²⁺) are required for RecA family recombinase proteins to exhibit RecA family recombinase activity and for exonucleases to exhibit exonuclease activity. Therefore, the reaction solution in which the assembly reaction is performed in the RA method contains a magnesium ion source. A magnesium ion source is a substance that provides magnesium ions in the reaction solution, for example, magnesium salts such as magnesium acetate [Mg(OAc)₂], magnesium chloride [MgCl₂], and magnesium sulfate [MgSO₄]. A preferred magnesium ion source is magnesium acetate.

The magnesium ion source concentration of the reaction solution in which the assembly reaction is performed in the RA method is not particularly limited, as long as the concentration is such that the RecA family recombinase protein can exhibit RecA family recombinase activity and the exonuclease can exhibit exonuclease activity. For example, 0.5 mM or more is preferred as the magnesium ion source concentration in the reaction solution at the start of the assembly reaction, and 1 mM or more is more preferred. On the other hand, if the magnesium ion concentration in the reaction solution is too high, the exonuclease activity may become too strong, and the assembling efficiency of multiple fragments may decrease. Therefore, for example, 20 mM or less is preferred, 15 mM or less is more preferred, 12 mM or less is even more preferred, and 10 mM or less is even more preferred as the magnesium ion source concentration in the reaction solution at the start of the assembly reaction.

The reaction solution in which the assembly reaction is performed in the RA method is prepared, for example, by adding a DNA fragment, a RecA family recombinase protein, an exonuclease, at least one of nucleoside triphosphate and deoxynucleotide triphosphate, and a magnesium ion source in buffer solution There are no restrictions on the buffer solution as long as it is suitable for use at pH 7-9, preferably pH 8. Examples are Tris-HCl, Tris-OAc, Hepes-KOH, phosphate buffer, MOPS-NaOH, Tricine-HCl, etc.

Preferred buffers are Tris-HCl or Tris-OAc. The concentration of the buffer solution can be appropriately selected by a person skilled in the art and is not particularly limited, but in the case of Tris-HCl or Tris-OAc, for example, 10 mM (mmol/L) to 100 mM, preferably 10 mM to 50 mM and more preferably a concentration of 20 mM can be selected.

It is preferred that the reaction solution in which the assembly reaction is performed in the RA method also contains nucleoside triphosphate or deoxynucleotide triphosphate regenerating enzyme and its substrate. The ability to regenerate nucleoside triphosphate or deoxynucleotide triphosphate in the reaction solution allows a large number of DNA fragments to be assembled more efficiently. The combinations of nucleoside triphosphate or deoxynucleotide triphosphate regenerating enzyme and its substrate include the combination of creatine kinase and creatine phosphate, pyruvate kinase and phosphoenolpyruvate, acetate kinase and acetyl phosphate, polyphosphate kinase and polyphosphate, nucleoside diphosphate kinase and nucleoside triphosphate. The nucleoside triphosphates that are substrates (phosphate sources) for nucleoside diphosphate kinase may be ATP, GTP, CTP, or UTP. Other regenerative enzymes include myokinases.

The reaction solution for the assembly reaction in the RA method can also contain alkali metal salts and reducing agents as described above for the RCR reaction. The reaction solution for the assembly reaction in the RA method can further contain a substance that suppresses the formation of secondary structures in single-stranded DNA and promotes specific hybridization (dimethyl sulfoxide (DMSO), tetramethylammonium chloride (TMAC), etc.), substances that have a macromolecular crowding effect (polyethylene glycol (PEG) 200-20000, polyvinyl alcohol (PVA) 200-2000, dextran 40-70, Ficoll 70, bovine serum albumin (BSA) etc.).

The present embodiment also relates to:
a kit for producing double-stranded circular DNA comprising:
   MutS,
   MutL,
   MutH and/or single-strand specific exonuclease,
a first enzyme group that catalyzes replication of circular DNA,
a second enzyme group that catalyzes an Okazaki fragment maturation and synthesizes two sister circular DNAs constituting a catenane; and
a third enzyme group that catalyzes a separation of two sister circular DNAs;
a kit for producing double-stranded circular DNA comprising:
   MutS,
   MutL,
   UvrD,
   single-strand specific exonuclease,
a first enzyme group that catalyzes replication of circular DNA;
a second enzyme group that catalyzes an Okazaki fragment maturation and synthesizes two sister circular DNAs constituting a catenane, and
a third enzyme group that catalyzes a separation of two sister circular DNAs; and
a kit for producing double-stranded circular DNA comprising:
   MutS,
      single-strand specific exonuclease,
      a first enzyme group that catalyzes replication of circular DNA;
a second enzyme group that catalyzes an Okazaki fragment maturation and synthesizes
two sister circular DNAs constituting a catenane; and
a third enzyme group that catalyzes a separation of two sister circular DNAs.

The above kit may contain all the components in a single kit or may not include some of the components if it is intended for use with the methods of the present embodiment. If the kit does not include some of the components, the user can add the necessary components to the kit when using it to implement the methods of the present embodiment.

The kit of the present embodiment may further include additional components for DNA assembly before amplification, such as a protein with RecA family recombinase activity, and optionally, additional components comprising exonucleases, at least one of nucleoside triphosphates and deoxynucleotides, and a source of magnesium ions. These additional components may be included in the kit of the present embodiment or provided as a separate kit intended to be used together with the kit of the present embodiment.

The specific components and concentrations of each component included in the kit of the present embodiment are as described above.

The kit of the present embodiment may include one package containing a mixture of the above components, or it may include packages containing the above components individually or mixed in several types. Preferably, the kit of the present embodiment includes the first enzyme group that catalyzes replication of circular DNA, the second enzyme group that catalyzes an Okazaki fragment maturation and synthesizes two sister circular DNAs constituting a catenane, and the third enzyme group that catalyzes a separation of two sister circular DNAs, with other components packaged separately. For example, it may be a kit that includes enzyme solution 1 containing MutS, MutL, and MutH (or enzyme solution 1′ containing MutS, MutL, UvrD, and single-strand specific exonuclease, or enzyme solution 1″ containing MutS and single-strand specific exonuclease), and enzyme solution 2 containing the first enzyme group that catalyzes replication of circular DNA, the second enzyme group that catalyzes an Okazaki fragment maturation and synthesizes two sister circular DNAs constituting a catenane, and the third enzyme group that catalyzes a separation of two sister circular DNAs.

The kit of the present embodiment may include instructions for implementing the methods of the present embodiment. The instructions may include the matters described above for the method of the present embodiment as an explanation.

The present embodiment also relates to the double-stranded DNA obtained by the methods of the present embodiment. This double-stranded DNA has a very low proportion of double-stranded DNA with sequence errors, which can enhance the accuracy in material productions via transcription, translation, etc., as well as in transformation processes in organisms such as E. coli. Due to the low proportion of double-stranded DNA with sequence errors, it is also useful as a template for mRNA or as a DNA memory for storing digital data.

### EXAMPLES

The invention is described in more detail in the following examples, but the invention is not limited by these examples.

### [Example 1] Effect of Adding a Mismatch Repair-Related Enzyme Group Before Amplification

In this example, we show that for a mixture of circular double-stranded DNA with mismatches and circular double-stranded DNA without mismatches, obtained by assembling DNA with mismatches, the action of a mismatch repair-related enzyme group before amplification suppressed the amplification of the circular double-stranded DNA with mismatches in the subsequent amplification reaction, thereby increasing the percentage of circular double-stranded DNA without mismatches.

1.4 kb DCW4 fragment (SEQ ID NO.1) from E. coli and the 1.3 kb DCW5oriC fragment (SEQ ID NO.2) are assembled by the RA assembly reaction using a 40 base pair overlap sequence added to both ends of each fragment, and the circular DNA pDCW4-5OriC (2.6 (2.6 kb) is obtained. In this case, it is possible to prepare GT mismatch-containing circular double-stranded DNA, etc., by using DCW4_GT fragment with base substitutions in the overlap sequence, etc., as shown in Table 3.

The DCW4 fragment of 5.65 nM, the DCW4_GT fragment of 5.65 nM and the DCW5oriC fragment of 11.3 nM (total final DNA concentration 5 ng/μL) were added to the RA assembly reaction solution consisting of the following composition and reacted at 42° C. for 30 min to perform RA assembly reaction, followed by RCR amplification reaction, and then heat treatment at 6° C. for 2 min. The assembly product containing circular DNA with GT mismatch and circular DNA without mismatch in equimolar concentrations was obtained. The respective sequences are shown in Tables 3-5 below.

Composition of RA assembly reaction solution: 1 μM wild-type RecA (prepared from an E. coli expression strain of RecA by purification with a process including polyethyleneimine precipitation, ammonium sulfate precipitation, and affinity column chromatography), 80 mU/μL of exonuclease III (2170A, TAKARA Bio), 1 U/μL of exonuclease I (M0293, New England Biolabs), 20 mM Tris-HCl (pH 8.0), 4 mM DTT, 1 mM magnesium acetate, 50 mM potassium glutamate, 100 μM ATP, 150 mM tetramethylammonium chloride (TMAC), 5 mass % PEG8000, 10 volume % DMSO, 20 ng/μL creatine kinase (10127566001, Sigma-Aldrich), 4 mM creatine phosphate. The concentration of each component in the RA reaction solution is the concentration relative to the total volume of the RA assembly reaction solution.

1 μL of the resulting circular DNA was added to the reaction buffer in Table 2 below (5 μL total) containing the mismatch repair-related enzyme groups listed in Table 1 below, and the reaction was further kept at 37° C. for 30 minutes for mismatch removal reaction.

TABLE 1

| Enzyme | composition |
|---|---|
| MutS | 300 nM MutS |
| MutS/L | 300 nM MutS; 300 nM MutL |
| MutS/UvrD | 300 nM MutS; 15 nM UvrD |
| MutS/L/UvrD | 300 nM MutS; 300 nM MutL; 15 nM UvrD |
| MutS/L/H | 300 nM MutS; 300 nM MutL; 100 nM MutH |

Each enzyme was prepared as follows.

MutS, MutL, and MutH were each massively expressed in E. coli as N-terminal histidine tag fused forms and then purified and prepared by a process involving affinity column chromatography. The molar concentrations are expressed as monomers.

UvrD was prepared from an E. coli expression strain by purification using a process involving affinity chromatography. The molar concentrations are expressed as monomers.

2.5 μL of the post-reaction sample was added to the RCR amplification reaction solution (5 μL total), and the RCR amplification reaction was carried out at 30° C. for 16 hours and supercoiling of the replication intermediate by finalization treatment (dilution of the reaction solution 5-fold with reaction buffer and further incubation at 33° C. for 30 minutes). 1 μL of the reaction was cleaved with restriction enzyme HindIII (0.5 U/μL) (total 5 μL), and a portion (1.5 μL) was analyzed by 1.2% agarose gel electrophoresis and SYBRGreen staining.

The RCR amplification reaction solution was a mixture of RCR reaction mixture consisting of the following composition and 30 nM Tus. Tus was prepared from an E. coli expression strain of Tus by a process including affinity column chromatography and gel filtration column chromatography.

TABLE 2
RCR Reaction mixture
Reaction buffer

| | | |
|---|---|---|
| Tris-HCl (pH 8.0) | 20 | mM |
| Dithiothreitol | 8 | mM |
| Potassium glutamate | 150 | mM |
| Mg(OAc)₂ | 10 | mM |
| Creatine phosphate | 4 | mM |
| ATP | 1 | mM |
| GTP, CTP, UTP | each 1 | mM |
| dNTPs | each 0.1 | mM |
| tRNA | 50 | ng/μL |
| NAD | 0.25 | mM |
| Ammonium sulfate | 10 | mM |
| Bovine serum albumin (BSA) | 0.5 | mg/mL |
| Creatine kinase | 20 | ng/μL |

Enzymes

| | | |
|---|---|---|
| SSB | 400 | nM |
| IHF | 20 | nM |
| DnaG | 400 | nM |
| DnaN | 40 | nM |
| PolIII* | 5 | nM |
| DnaB, DnaC | 20 | nM |
| DnaA | 100 | nM |
| RNaseH | 10 | nM |
| Ligase | 50 | nM |
| PolI | 50 | nM |
| GyrA, GyrB | 50 | nM |
| Topo IV | 5 | nM |
| Topo III | 50 | nM |
| RecQ | 50 | nM |

In Table 2, SSB represents SSB from Escherichia coli, IHF represents a complex of IhfA and IhfB from Escherichia coli, DnaG represents DnaG from Escherichia coli, DnaN represents DnaN from Escherichia coli, Pol III* represents the DNA polymerase III* complex from Escherichia coli consisting of DnaX, HolA, HolB, HolC, HolD, DnaE, DnaQ, and HolE, DnaB represents DnaB from Escherichia coli, DnaC represents DnaC from Escherichia coli, DnaA represents DnaA from Escherichia coli, RNaseH represents RNaseH from Escherichia coli, Ligase represents DNA ligase from Escherichia coli, Pol I represents DNA polymerase I from Escherichia coli, GyrA represents GyrA from Escherichia coli, GyrB represents GyrB from Escherichia coli, Topo IV represents the complex of ParC and ParE from Escherichia coli, Topo III represents topoisomerase III from Escherichia coli, and RecQ represents RecQ from Escherichia coli.

SSB was prepared and purified from Escherichia coli expression strains through ammonium sulfate precipitation and ion exchange column chromatography.

IHF was prepared and purified from co-expression strains of IhfA and IhfB from Escherichia coli through ammonium sulfate precipitation and affinity column chromatography.

DnaG was prepared and purified from Escherichia coli expression strains through ammonium sulfate precipitation, anion exchange column chromatography, and gel filtration column chromatography.

DnaN was prepared and purified from Escherichia coli expression strains through ammonium sulfate precipitation and anion exchange column chromatography.

Pol III* was prepared and purified from Escherichia coli co-expression strains of DnaX, HolA, HolB, HolC, HolD, DnaE, DnaQ, and HolE through ammonium sulfate precipitation, affinity column chromatography, and gel filtration column chromatography.

DnaB and DnaC were prepared and purified from Escherichia coli co-expression strains of DnaB and DnaC through ammonium sulfate precipitation, affinity column chromatography, and gel filtration column chromatography.

DnaA was prepared and purified from Escherichia coli expression strains through ammonium sulfate precipitation, dialysis precipitation, and gel filtration column chromatography.

GyrA and GyrB were prepared and purified from a mixture of Escherichia coli expression strains of GyrA and GyrB through ammonium sulfate precipitation, affinity column chromatography, and gel filtration column chromatography.

Topo IV was prepared and purified from a mixture of Escherichia coli expression strains of ParC and ParE through ammonium sulfate precipitation, affinity column chromatography, and gel filtration column chromatography.

Topo III was prepared and purified from Escherichia coli expression strains through ammonium sulfate precipitation and affinity column chromatography.

RecQ was prepared and purified from Escherichia coli expression strains through ammonium sulfate precipitation, affinity column chromatography, and gel filtration column chromatography.

RNaseH, Ligase, and Pol I were commercial enzymes derived from Escherichia coli (Takara Bio).

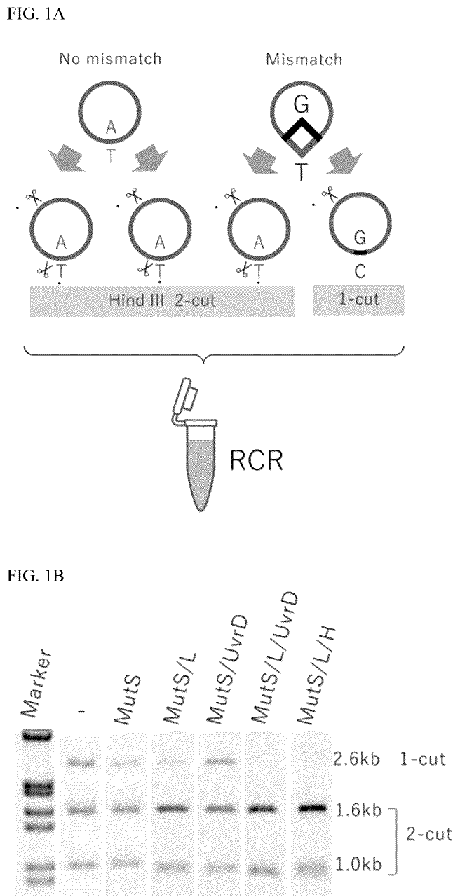

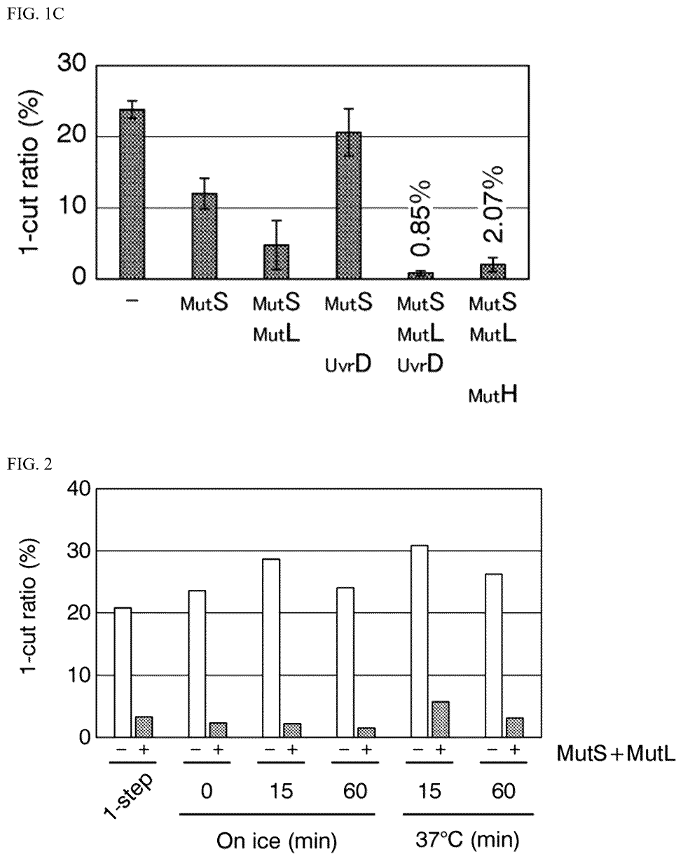

A schematic of the experiment is shown in FIG. 1A. When circular DNA with mismatch and circular DNA without mismatch are present in equimolar concentrations, each strand of double-stranded circular DNA amplifies when amplified by the RCR method, producing circular DNA with base substitutions derived from the mismatch in 25% of the circular DNA. The double-stranded circular DNA was designed to have two restriction enzyme HindIII recognition sequences in the absence of mismatches, one of which was designed as a mismatch site. This resulted in circular DNA without mismatch being cleaved at two sites and detected as two DNA fragments (1.6 kb and 1.0 kb in the case of pDCW4-5OriC), while circular DNA with mismatch-derived base substitutions was cleaved at only one site and detected as one DNA fragment (it is detected as a mismatch-derived fragment. 2.6 kb in the case of pDCW4-5OriC).

The five enzymes listed in Table 1 were used as the mismatch repair-related enzyme group, and the results of DNA fragment detection by agarose gel electrophoresis after RCR amplification and restriction enzyme cleavage are shown in FIG. 1B. The one without mismatch repair-related enzymes was used as control.

From the results of the electrophoresis in FIG. 1B, the ratio of the band intensity of the mismatch-derived fragment (2.6 kb) to the total band intensity (1-cut ratio) was quantified and graphed in FIG. 1C. In the sample without mismatch repair-related enzymes (−), about 25% of mismatch-derived fragments were observed, as expected from FIG. 1A, whereas the addition of MutS reduced the percentage of mismatch-derived fragments to 12%, and the addition of MutL in addition to MutS reduced the percentage to 5%. The addition of UvrD or MutH to the system containing MutS and MutL reduced the percentage of mismatch-derived fragments to 0.85% or 2%, respectively. The decrease in the percentage of double-stranded DNA with mismatches/sequence errors and the increase in the percentage of double-stranded DNA without mismatches/sequence errors is hereinafter also referred to as the mismatch removal effect or sequence error removal effect.

TABLE 3

| Base substitutions introduced into the overlapping sequence at 5′ | | |
|---|---|---|
| end of DCW4 fragment (Underline: HindIII recognition sequence of | | SEQ |
| its modified sequence. Uppercase: Base substitution) | | ID NO. |
| No mismatch | ggcttagtgaaacaaagcttcagtcccgtactctacgcgc | 3 |
| (DCW4) | | |
| GT mismatch | ggcttagtgaaacaGagcttcagtcccgtactctacgcgc | 4 |
| (DCW4_GT) | | |

TABLE 4
DCW4 fragment (1.4 kb) (SEQ ID NO. 1) (Lowercase: overlapping sequence)

ggcttagtgaaacaaagcttcagtcccgtactctacgcgcCAGAGGGAAATTCACCTGGCGCGTATTTTTGTTCGCGGCT
TAGCTACGGCTGAGCACGCCGCGGCGATAGAGATTACGTTTAATGCGCGTTAAACCAGGTTTTGGCTTACGCGGTTCGTC
CAGGCTTGCCAGGACAATCTCCAGCACACGCTCTGCGACATCGCGGTGACGTTGAGCCACTGCCAGCACCGGACACTGTA
AGAAGTCGAGCAGTTCGTTATCGCCAAAGGTGGCAATTGCCAGGTCAGAAGGCAGTTTGCCGTCGCGACGCAGCGTGACA
TCCATCACTCCTTGCAACAACGCAAACGACGTTGTGAACAGCGCCTGCGGCATCGGATGCGTTTCCAGCCATTTTTCGAA
TAACTGGGCAGCCGCCTCCCGCTCATAGCTGTTGGCATACAGGAAATGCACTTCGCGCGGATCATCTTTCCAGGCAGTAC
GGAAACCTTGTTCACGCAGGAAGCTGACAGAAAGCTCCGGTAGCGCACCAAGATAAAGCACCGTCTCGGGGGGAAACTTA
CGTAACTCTTCCGCCAGCATTTCGGCATCATCCTGATCGGCACCAACCACGCTGGTGAAGTGTTCACGATCGAGGGCGCG
GTCCAGCGCGACAATCGGGAACGGGTCGTTAGCCCAGCGTTGATAAAAAGGATGCTCAGGAGGCAACGACGTCGAAACAA
TAATGGCATCAACCTGACGCTGTAAAAGGTGCTCAATGCACCGCATTTCGTTGTCTGGCTGATCTTCTGAGCAGGCAATC
AGGAGTTGATAACCCCGTTGCCGCGCCTGGCGTTCAAGATAGTTAGCGATGCGGGTATAGCTGGTGTTCTCCAGATCGGG
GATCACAAGACCAATAGAACGTGTGCGTCCAGCACGAAGCCCAGGTGCCACGGCGTTCGGGTGGTAATTGTGCTCACGCA
CCACAGCCATGACTTTTTCAACGGTTTTGTCGCTCACACGGTATTGCTTCGCTTTGCCGTTAATAACATAGCTTGGAGTG
GTCCGCGACACTCCCGCCAGCCGAGCGATTTCATCCAGTTTCACAATTGCCCCTTGCGTAAAATGTAAAAACCATAGCCA
TTGTACTGGTATGGGTTAAATTCATTAACATCTAAGCGCAGAAAATTAACTGCGGCAACTGCTTTTATTCCAGGTTCCCA
CTGATTTCGCAAAAAAAGCCCAACGTGACATGTTGGGCTGTAAATTGCGCATTGAGATCATTCTCAACGCATTATTTTAT
CGCCGCGCGAAAGTCCGACCACACCAGAGCGAGCAACCTCCACAATTTTCgccacatcgcgaatcgatgctttatcaaac
gtgcgcacag

TABLE 5
DCW5oriC fragment (1.3 kb) (SEQ ID NO. 2) (Lowercase: overlapping sequence)

gccacatcgcgaatcgatgctttatcaaacgtgcgcacagTCCATCCAGAGGGCTTTACAGTGTACATATCAACCCATAT
TCGGCTTGAGAAAGACCTGAGTATGTTGTAACTAAAGGGATCCTGGGTATTAAAAAGAAGATCTATTTATTTAGAGATCT
GTTCTATTGTGATCTCTTATTAGGATCGGACTGCCCTGTGGATAACAAGGATCCGGCTTTTAAGATCAACAACCTGGAAA
GGATCATTAACTGTGAATGATCGGTGATCCTGGACCGTATAAGCTGGGATCAGAATGAGGGGTTATACACAACTCAAAAA
CTGAACAACAGTTGTTCTTTGGATAACTACCGGTTGATCCAAGCTTCCTGACAGAGTTATCCACAGTAGATCGCACGATC
TGTATAACTTCGTATAGCATACATTATACGAAGTTATCTTTAGTTACAACATACTCCATTCTTCTGCCGGATCTTCTTAC
GCCCCGCCCTGCCACTCATCGCAGTACTGTTGTAATTCATTAAGCATTCTGCCGACATGGAAGCCATCACAGACGGCATG
ATGAACCTGAATCGCCAGCGGCATCAGCACCTTGTCGCCTTGCGTATAATATTTGCCCATGGTGAAAACGGGGGCGAAGA
AGTIGTCCATATTGGCCACGTTTAAATCAAAACTGGTGAAACTCACCCAGGGATTGGCTGAGACGAAAAACATATTCTCA
ATAAACCCTTTAGGGAAATAGGCCAGGTTTTCACCGTAACACGCCACATCTTGCGAATATATGTGTAGAAACTGCCGGAA
ATCGTCGTGGTATTCACTCCAGAGCGATGAAAACGTTTCAGTTTGCTCATGGAAAACGGTGTAACAAGGGTGAACACTAT
CCCATATCACCAGCTCACCGTCTTTCATTGCCATACGGAATTCCGGATGAGCATTCATCAGGGGGGCAAGAATGTGAATA
AAGGCCGGATAAAACTTGTGCTTATTTTTCTTTACGGTCTTTAAAAAGGCCGTAATATCCAGCTGAACGGTCTGGTTATA
GGTACATTGAGCAACTGACTGAAATGCCTCAAAATGTTCTTTACGATGCCATTGGGATATATCAACGGTGGTATATCCAG
TGATTTTTTTCTCCATTTTAGCTTCCTTAGCTCCTGAAAATCTCGATAACTCAAAAAATACGCCCGGTAGTGATCTTATT
TCATTATGGTGAAAGTTGGAACCTCTTACGCTCATTCCCTTCTTTATCGGGTTACAGAACCGGTTTACGCAGTTTCggct
tagtgaaacaaagcttcagtcccgtactctacgcgc

### [Example 2] Temperature During Addition of Mismatch Repair-Related Enzymes and Mismatch Removal Reaction During Amplification

The temperature conditions for the mismatch removal reaction in Example 1 were examined. Specifically, the mismatch removal reaction using 300 nM MutS and 300 nM MutL in Example 1 was performed under different temperature conditions: 0 min, 15 min or 60 min on ice, or 15 min or 60 min at 37° C. A separate system was examined where the mismatch repair-related enzyme group was not added before the DNA amplification reaction (mismatch removal reaction before amplification was not performed), and the mismatch repair-related enzyme group brought into the amplification reaction was added to the RCR amplification reaction solution (2.5 μL) in advance. 2.5 μL of RA joining product diluted 5-fold with the reaction buffer of Table 2 was directly added to the said RCR amplification reaction solution. (The final concentrations of the mismatch repair-related enzymes are 150 nM MutS and 150 nM MutL.) In this system, the mismatch removal reaction and circular DNA amplification reaction are performed in one reaction (1-step). All other steps were performed as in Example 1, and samples were electrophoresed after restriction enzyme cleavage of the RCR amplification product. For each temperature and reaction time, those without the addition of the mismatch repair-related enzyme group were used as controls. The ratio of the band intensity of mismatch-derived fragments to the overall band intensity (1-cut ratio) was quantified and graphed in FIG. 2.

As shown in FIG. 2, the 1-cut ratio, which indicates the percentage of mismatch-derived fragments, was lower when MutS and MutL were added (+) compared to when they were not added (−) at all temperature and time conditions, confirming the mismatch removal effect. Furthermore, when MutS and MutL were added directly to the RCR amplification reaction solution and the mismatch removal reaction and RCR amplification reaction were performed in a single step, the 1-cut ratio was reduced by the same degree. The mismatch removal effect was also observed when the mismatch removal reaction was performed on ice for 0 min, suggesting that the mismatch removal reaction can be performed simultaneously during the RCR amplification reaction even if the mismatch repair-related enzyme group from the mismatch removal reaction is brought into the RCR amplification reaction.

### [Example 3] Effect of Sequence Error Removal on Double-Stranded DNA with Various Sequence Errors

In Example 1, the effect of sequence error removal on each of the circular double-stranded DNA with different mismatches or single nucleotide insertions was examined in a reaction system using MutS, MutL, and UvrD as mismatch repair-related enzyme groups before DNA amplification. Specifically, instead of the circular DNA with mismatch used in Example 1 (a combination of the DCW4_GT fragment with a GT mismatch at the AT position and the DCW5oriC fragment (SEQ ID NO. 2) 1.3 kb), the DCW4-1 fragment with a single base substitution in the overlap sequence (SEQ ID NO. 5) 0.65 kb and DCW5oriC-1 fragment (SEQ ID NO. 6) 1.9 kb (with an overlap of 46 or 60 bases with each other). The respective sequences are shown in Tables 6-8 below.

TABLE 6

| Base substitutions introduced into the overlapping sequence at 5′ end of | | |
|---|---|---|
| DCW4-1 fragment (Underline: HindIII recognition sequence of its modified | | SEQ |
| sequence. Uppercase: Base substitution) | | ID NO. |
| No mismatch | gtgctggatctgcccggagatccccaccgccaggtaccttaaaagctt | 7 |
| (DCW4-1_AT) | cagcatcaggtt | |
| AC imismatch | gtgctggatctgcccggagatccccaccgccaggtaccttaaaagcCt | 8 |
| (DCW4-1_AC) | cagcatcaggtt | |
| AG mismatch | gtgctggatctgcccggagatccccaccgccaggtaccttaaaagcGt | 9 |
| (DCW4-1_AG) | cagcatcaggtt | |
| CT imismatch | gtgctggatctgcccggagatccccaccgccaggtaccttaaCagctt | 10 |
| (DCW4-1_CT) | cagcatcaggtt | |
| GT mismatch | gtgctggatctgcccggagatccccaccgccaggtaccttaaGagctt | 11 |
| (DCW4-1_GT) | cagcatcaggtt | |
| AA mismatch | gtgctggatctgcccggagatccccaccgccaggtaccttaaaagcAt | 12 |
| (DCW4-1_AA) | cagcatcaggtt | |
| CC mismatch | gtgctggatctgcccggagatccccaccgccaggtaccttaaaaCctt | 13 |
| (DCW4-1_CC) | cagcatcaggtt | |
| TT mismatch | gtgctggatctgcccggagatccccaccgccaggtaccttaaTagctt | 14 |
| (DCW4-1_TT) | cagcatcaggtt | |
| GG mismatch | gtgctggatctgcccggagatccccaccgccaggtaccttaaaagGtt | 15 |
| (DCW4-1_GG) | cagcatcaggtt | |

TABLE 7
DCW4-1 fragment (0.65 kb) (SEQ ID NO. 5) (Lower case: overlapping sequence)

gtgctggatctgcccggagatccccaccgccaggtaccttaaaagcttcagcatcaggttGCATCGGATGCGTTTCCAGC
CATTTTTCGAATAACTGGGCAGCCGCCTCCCGCTCATAGCTGTTGGCATACAGGAAATGCACTTCGCGCGGATCATCTTT
CCAGGCAGTACGGAAACCTTGTTCACGCAGGAAGCTGACAGAAAGCTCCGGTAGCGCACCAAGATAAAGCACCGTCTCGG
CGGGAAACTTACGTAACTCTTCCGCCAGCATTTCGGCATCATCCTGATCGGCACCAACCACGCTGGTGAAGTGTTCACGA
TCGAGGGCGCGGTCCAGCGCGACAATCGGGAACGGGTCGTTAGCCCAGCGTTGATAAAAAGGATGCTCAGGAGGCAACGA
CGTCGAAACAATAATGGCATCAACCTGACGCTGTAAAAGGTGCTCAATGCACCGCATTTCGTTGTCTGGCTGATCTTCTG
AGCAGGCAATCAGCAGTTGATAACCCCGTTGCCGCGCGTGGCGTTCAAGATAGTTAGCGATGGGGGTATAGCTGGTGTTC
TCCAGATCGGGGATCACAAGACCAATAGAACGTGTGCGTCCAGCAcgaagcccagctgccacggcgttcgggtggtaatt
gtgctcacgca

TABLE 8
DCW5oriC-1 fragment (1.9 kb) (SEQ ID NO. 6) (Lower case: overlapping sequence)

cgaagcccagctgccacggcgttcgggtggtaattgtgctcacgcaTTTATCAAACGTGCGCACAGTCCATCCAGAGGGC
TTTACAGTGTACATATCAACCCATATTCGGCTTGAGAAAGACCTGAGTATGTTGTAACTAAAGGGATCCTGGGTATTAAA
AAGAAGATCTATTTATTTAGAGATCTGTTCTATTGTGATCTCTTATTAGGATCGCACTGCCCTGTGGATAACAAGGATCC
GGCTTTTAAGATCAACAACCTGGAAAGGATCATTAACTGTGAATGATCGGTGATCCTGGACCGTATAAGCTGGGATCAGA
ATGAGGGGTTATACACAACTCAAAAACTGAACAACAGTTGTTCTTTGGATAACTACCGGTTGATCCAAGCTTCCTGACAG
AGTTATCCACAGTAGATCGCACGATCTGTATAACTTCGTATAGCATACATTATACGAAGTTATCTTTAGTTACAACATAC
TCCATTCTTCTGCCGGATCTTCTTACGCCCCGCCCTGCCACTCATCGCAGTACTGTTGTAATTCATTAAGCATTCTGCCG
ACATGGAAGCCATCACAGACGGCATGATGAACCTGAATCGCCAGCGGCATCAGCACCTTGTCGCCTTGCGTATAATATTT
GCCCATGGTGAAAACGGGGGCGAAGAAGTTGTCCATATTGGCCACGTTTAAATCAAAACTGGTGAAACTCACCCAGGGAT
TGGCTGAGACGAAAAACATATTCTCAATAAACCCTTTAGGGAAATAGGCCAGGTTTTCACCGTAACACGCCACATCTTGC
GAATATATGTGTAGAAACTGCCGGAAATCGTCGTGGTATTCACTCCAGAGCGATGAAAACGTTTCAGTTTGCTCATGGAA
AACGGTGTAACAAGGGTGAACACTATCCCATATCACCAGCTCACCGTCTTTCATTGCCATACGGAATTCCGGATGAGCAT
TCATCAGGCGGGCAAGAATGTGAATAAAGGCCGGATAAAACTTGTGCTTATTTTTCTTTACGGTCTTTAAAAAGGCCGTA
ATATCCAGCTGAACGGTCTGGTTATAGGTACATTGAGCAACTGACTGAAATGCCTCAAAATGTTCTTTACGATGCCATTG
GGATATATCAACGGTGGTATATCCAGTGATTTTTTTCTCCATTTTAGCTTCCTTAGCTCCTGAAAATCTCGATAACTCAA
AAAATACGCCCGGTAGTGATCTTATTTCATTATGGTGAAAGTTGGAACCTCTTACGCTCATTCCCTTCTTTATCGGGTTA
CAGAACCGGTTTACGCAGTTTCGGCTTAGTGAAACAAAGAAAAGTGTTTGTTAAATCGGCAATTCATCGCCGCATGTAAC
AAAATAATGCAAAAACGCGCTGTCGATATCACTTAATTATTGTGAATTTCAGCTGGTGAGTTAATTAACGGGGGGGTTTA
AAAGCATTTATCAATAATAGTCAGGAATAGTCTTATTTACTTTAAGCATATTGATGTCCAGTCCCGTACTCTACGCGCCA
GAGGGAAATTCACCTGGCGCGTATTTTTGTTCGCGGCTTAGCTACGGCTGAGCACGCCGCGGCGATAGAGATTACGTTTA
ATGCGCGTTAAACCAGGTTTTGGCTTACGCGGTTCGTCCAGGCTTGCCAGGACAATCTCCAGCACACGCTCTGCGACATC
GCGGTGACGTTGAGCCACTGCCAGCACCGGACACTGTAAGAAGTCGAGCAGTTCGTTATCGCCAAAGGTGGCAATTGCCA
GGTCAGAAGGCAGTTTGCCGTCGCGACGCAGCGTGACATCCATCACTCCTTGCAACAACGCAAACGACGTTGTGAACAGC
GCCTGCGAGCCGCCTCCCGCTCATAGCgtgctggatctgcccggagatccccaccgccaggtaccttaaaagcttcagca
tcaggtt

Similarly, the effect of sequence error removal was examined for circular DNA with a single base insertion sequence error in a reaction system in which MutS, MutL, and UvrD were used as a mismatch repair-related enzyme group added before DNA amplification. 1.4 kb of the DCW4 fragment (SEQ ID NO. 1) of Example 1 with a single base insertion in the overlap sequence; and 1.3 kb of DCW5oriC fragment (SEQ ID NO. 2) were used for the single base insertion. The sequences used for the single nucleotide insertion are shown in Table 9 below.

TABLE 9

| Base insertions introduced into the overlapping sequence at 5′ end | | |
|---|---|---|
| of DCW4 fragment (Underline: HindIII recognition sequence of its | | SEQ |
| modified sequence. Uppercase: Base insertion) | | ID NO. |
| No sequence error | ggcttagtgaaacaaagcttcagtcccgtactctacgcgc | 3 |
| (DCW4) | | |
| Aᵢₙₛ (A insertion_DCW4_Ai) | ggcttagtgaaacaaagActtcagtcccgtactctacgcgc | 16 |
| Cᵢₙₛ (C insertion_DCW4_Ci) | ggcttagtgaaacaaagCcttcagtcccgtactctacgcgc | 17 |
| Gᵢₙₛ (G insertion_DCW4_Gi) | ggcttagtgaaacaaagGcttcagtcccgtactctacgcgc | 18 |
| Tᵢₙₛ (T insertion_DCW4_Ti) | ggcttagtgaaacaaagTcttcagtcccgtactctacgcgc | 19 |

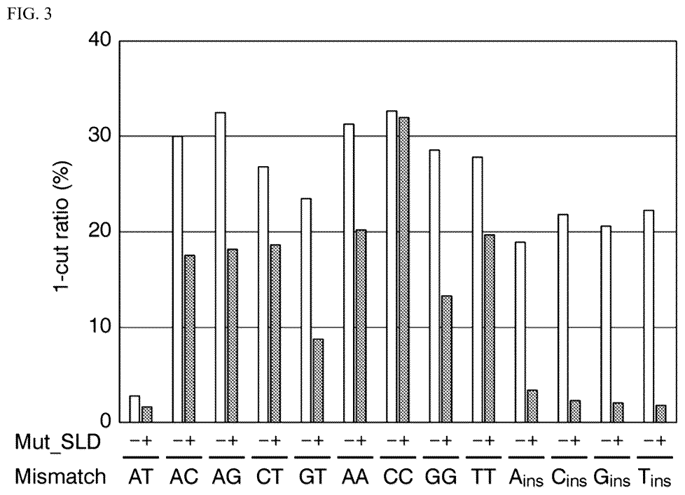

The mismatch repair reaction was performed as in Example 1, except that each DNA fragment used in the assembly reaction was 1/11.3 of the volume of Example 1, and the 0.5 μL of the reactant was added to the 5 μL mismatch removal reaction. The samples were electrophoresed after restriction enzyme cleavage of the RCR amplified product. The ratio of the band intensity of DNA fragments with sequence errors to the overall band intensity (1-cut ratio) was quantified and graphed in FIG. 3.

As shown in FIG. 3, for all sequence errors, although to varying degrees, the 1-cut ratio, which indicates the percentage of DNA with sequence errors, decreased when the mismatch repair-related enzyme group was used, confirming the effectiveness of sequence error removal. These effects correlate with the reported affinity of MutS for each mismatch pair, and in that report (Brown J. et al., Biochem. 2001, vol. 354, p. 627-633), MutS was shown to have mismatch-specific affinity for the CC mismatch, which was less effective in this example. Therefore, it was thought that the effect of sequence error removal on various sequence errors could be further enhanced by adjusting the amount and type of mismatch repair-related enzyme groups and reaction conditions.

### [Example 4] Increased Sequence Error Removal by Single-Strand Specific Exonuclease

We found that the addition of a single-strand-specific exonuclease to the mismatch repair-related enzyme group (MutS, MutL, and UvrD) used in Example 3 increased the effectiveness of sequence error removal.

Two overlap assembly reactions were used: the RA assembly reaction, in which the 5′ overhang strands hybridize and assemble together; and USER (registered trademark) Cloning (NEB), in which the 3′ overhang strands hybridize and assemble together. Circular DNA with sequence errors was prepared using each of the assembly reactions, and various single-strand-specific exonucleases were added to the sequence error removal reactions. The following four single-strand specific exonucleases were used.
RecJ: 5′=>3′ direction, degrading nucleotides from the 5′ end
Exonuclease I (ExoI): Degrades nucleotides from the 3′ end in the 3′→5′ direction
Exonuclease T (ExoT): Degrades nucleotides from the 3′ end in the 3′→5′ direction
Exonuclease VII (ExoVII): degrades nucleotides in both directions from both the 3′ and 5′ ends

RA assembly reactions were performed in the same manner as in Example 1 (5′ overhang) using the 6 nM UPL fragment 2.8 kb (SEQ ID NO. 20) and 6 nM UPR fragment 2.0 kb (SEQ ID NO. 21) shown in Tables 12 and 13. Sequence errors were introduced using UPR_GN shown in Table 10 (N=mixed bases of A, T, C and G. Percentage of mismatch DNA generated: 75%).

TABLE 10

| Base substitutions introduced into the overlapping sequence at 5′ end | | |
|---|---|---|
| of UPR fragment (Underline: NruI recognition sequence of its | | SEQ |
| modified sequence. Uppercase: Base substitution) | | ID NO. |
| No mismatch | aacagccctgctgcgcggttcgcgacataatgttcccgat | 22 |
| (UPR) | | |
| GN mismatch | aacagccctgctgcgcggttNgcgacataatgttcccgat | 23 |
| (UPR_GN) | | |

For USER (registered trademark) Cloning, a 6 nM UPL-U fragment of 2.8 kb, a 4.5 nM UPR-U_GT fragment of 2.0 kb, and a 1.5 nM UPR-U fragment of 2.0 kb were added to CutSmart buffer (NEB) containing 20 mU/μl Thermolabile USER II Enzyme (NEB) in CutSmart buffer (NEB) (5 μL total), followed by warming at 37° C. for 30 min, heat treatment at 75° C. for 5 min and slow cooling (0.1° C./sec) (3′ overhang).

Each fragment was prepared by PCR using the template and dU-containing primer pairs shown in Table 11, respectively.

TABLE 11

| Underline: NruI recognition sequence of its modified sequence. | | | SEQ |
|---|---|---|---|
| Uppercase: Base substitution and dU | | | ID NO. |
| UPR- | template | UPR fragment | 21 |
| U fragment | primer | aacagcccUgcagcgcggUtCgcgacaUaatgttcccgaUtc | 24 |
| | | atgttctttccUgcgttatccccUgattctgtggataaccgUattac | 25 |
| UPR- | template | UPR fragment | 21 |
| U_GT | primer | aacagcccUgcagcgcggUtAgcgacaUaatgttcccgaUtc | 26 |
| fragment | | atgttctttccUgcgttatccccUgattctgtggataaccgUattac | 25 |
| UPL- | template | UPL fragment | 20 |
| U fragment | primer | acggttaUccacagaaUcaggggaUaacgcaggaaagaacaUg | 27 |
| | | atcgggaacattatgUcgcgaaccgcgcUgcagggctgtUg | 28 |

TABLE 12
UPL fragment (2.8 kb) (SEQ ID NO. 20)
(Lower case: overlapping sequence, Underline: Adapter sequence for Illumina sequencing)

gcggtaatacggttatccacagaatcaggggataacgcaggAAAGAACATGTGAGCAAAAGGCCAGCAAAAGGCCAGGAA
CCGTAAAAAGGCCGCGTTGCTGGCGTTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTGGCGCTTTCTGAATGGTCACGCTGTAGGTA
TCTCAGTTCGGTGTAGGTCGTTCGCTCCAAGCTGGGCTGTGTGCACGAACCCCCCGTTCAGCCCGACCGCTGCGCCTTAT
CCGGTAACTATCGTCTTGAGTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCAAGAAGATCCTTTGATAAAAAAACAG
CCTCCGAAAGGAGGCTGTAGGCAAATATTCTAGTATGTTGTACCTAAAGGAAATGAGCTGACAGATCGTGCGATCTACTG
TGGATAACTCTGTCAGGAAGCTTGGATCAACCGGTAGTTATCCAAAGAACAACTGTTGTTCAGTTTTTGAGTTGTGTATA
ACCCCTCATTCTGATCCCAGCTTATACGGTCCAGGATCACCGATCATTCACAGTTAATGATCCTTTCCAGGTTGTTGATC
TTAAAAGCCGGATCCTTGTTATCCACAGGGCAGTGCGATCCTAATAAGAGATCACAATAGAACAGATCTCTAAATAAATA
GATCTTCTTTTTAATACCCAGGATCCCTTTAGGTACAACATACTGTTTGGGATTCACTGAGTTTTAAATCAATCTAAAGT
ATATATGAGTAAACTTGGTCTGACAGTTACCAATGCTTATCTTTTCTACGGGGTCTGACGCTCAGTGGAACGAAAACTCA
CGTTAAGGGATTTTGGTCATGAGATTATCAAAAAGGATCTTCACCTAGATCCTTTTAAATTAAAAATGAAGTTTTAAATC
AATCTAAAGTATATATGAGTAAACTTGGTCTGACATGTCAGGAAGTTACCAATGCTTAATCAGTGAGGCACCTATGTCAG
CGATCTGTCTATTTCGTTCATCCATAGTTGCCTGACTCCCCGTCGTGTAGATAACTACGATACGGGAGGGCTTACCATCT
GGCCCCAGTGCTGCAATGATACCGCGAGACCCACGCTCACCGGCTCCAGATTTATCAGGAATAAACCAGCCAGCCGGAAG
GGCCGAGCGCAGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGCTACAGGCATCGTGGTGTCACGCTCGTCGTTTGGTATGGCT
TCATTCAGCTCCGGTTCCCAACGATCAAGGCGAGTTACATGATCCCCCATGTTGTGCAAAAAAGGGGTTAGCTCCTTCGG
TCCTCCGATCGTTGTCAGAAGTAAGTTGGCCGCAGTGTTATCACTCATGGTTATGGCAGCACTGCATAATTCTCTTACTG
TCATGCCATCCGTAAGATGCTTTTCTGTGACTGGTGAGTACTCAACCAAGTCATTCTGAGAATAGTGTATGCGGCGACCG
AGTTGCTCTTGCCCGGCGTCAATACGGGATAATACCGCGCCACATAGCAGAACTTTAAAAGTGCTCATCATTGGAAAACG
TTCTTCGGGGGGAAAACTCTCAAGGATCTTACCGCTGTTGAGATCCAGTTCGATGTAACCCACTCGTGCACCCAACTGAT
CTTCAGCATCTTTTACTTTCACCAGCGTTTCTGGGTGAGCAAAAACAGGAAGGCAAAATGCCGCAAAAAAGGGAATAAGG
GCGAGACGGAAATGTTGAATACTCATACTCTTCCTTTTTCAATATTATTGAAGCATTTATCAGGGTTATTGTCTCATGAG
CGGATACATATTTGAATGTATTTAGAAAAATAAACAAATAGGGGTTCCGCGCACATTTCCCCGAAAAGTGCCACCTGACG
TCTAAGAAACCATTATTATCATGACATTAACCTATAAAAATAGGCGTATCACGAGGCCCTTTCGTCTCGCGCGTTTCGGT
GATGACGGTGAAAACCTCTGACACATGCAGCTCCCGGAGACGGTCACAGCTTGTCTGTAAGGGGATGCCGGGAGCAGACA
AGCCCGTCAGGGCGCGTCAGCGGGTGTTGGCGGGTGTCGGGGCTGGCTTAACGGTCTCCAATGATACGGCGACCACCGAG
ATCTACACGCGTAAGATCGTCGGCAGCGTCAGATGTGTATAAGAGACAGTGTTCGGTATTGGTGCAGCGGTTCTGGTTTA
TTTCGGGATTAAAAAACTGAAAAAAGGGGGGGGCAGTTCCTGGTTACGTGACCTGATTTACTGGTATATGCCaacagccc
tgctgcgcggttcgcgacataatgttcccgat

TABLE 13
UPR fragment (2.0 kb) (SEQ ID NO. 21)
(Lower case: overlapping sequence, Underline: Adapter sequence for Illumina sequencing)

aacagccctgctgcgcggttcgcgacataatgttcccgatTCGTGTTTCCGGCAGTGGATTAAATAAAACTGCTGTCTCT
TATACACATCTCCGAGCCCACGAGACAAGAGGCAATCTCGTATGCCGTCTTCTGCTTGCGAGACCATGCGGCATCAGAGC
AGATTGTACTGAGAGTGCACCATATGCGGTGTGAAATACCGCACAGATGCGTAAGGAGAAAATACCGCATCAGGCGCCAT
TCGCCATTCAGGCTGCGCAACTGTTGGGAAGGGCGATCGGTGCGGGCCTCTTCGCTATTACGCCAGCTGGCGAAAGGGGG
ATGTGCTGCAAGGCGATTAAGTTGGGTAACGCCAGGGTTTTCCCAGTCACGACGTTGTAAAACGACGGCCAGTGAATTCC
CCGGATCCGTCGACCTGCAGGGGGGGGGGGGCGCTGAGGTCTGCCTCGTGAAGAAGGTGTTGCTGACTCATACCAGGCCT
GAATCGCCCCATCATCCAGCCAGAAAGTGAGGGAGCCACGGTTGATGAGAGCTTTGTTGTAGGTGGACCAGTTGGTGATT
TTGAACTTTTGCTTTGCCACGGAACGGTCTGCGTTGTCGGGAAGATGCGTGATCTGATCCTTCAACTCAGCAAAAGTTCG
ATTTATTCAACAAAGCCGCCGTCCCGTCAAGTCAGCGTAATGCTCTGCCAGTGTTACAACCAATTAACCAATTCTGATTA
GAAAAACTCATCGAGCATCAAATGAAACTGCAATTTATTCATATCAGGATTATCAATACCATATTTTTGAAAAAGCCGTT
TCTGTAATGAAGGAGAAAACTCACCGAGGGAGTTCCATAGGATGGCAAGATCCTGGTATCGGTCTGCGATTCCGACTGGT
CCAACATCAATACAACCTATTAATTTCCCCTCGTCAAAAATAAGGTTATCAAGTGAGAAATCACCATGAGTGACGACTGA
ATCCGGTGAGAATGGCAAAAGCTTATGCATTTCTTTCCAGACTTGTTCAACAGGCCAGCCATTACGCTCGTCATCAAAAT
CACTCGCATCAACCAAACCGTTATTCATTCGTGATTGCGCCTGAGCGAGACGAAATACGCGATCGCTGTTAAAAGGACAA
TTACAAACAGGAATCGAATGCAACCGGCGCAGGAACACTGCCAGCGCATCAACAATATTTTCACCTGAATCAGGATATTC
TTCTAATACCTGGAATGCTGTTTTCCCGGGGATCGCAGTGGTGAGTAACCATGCATCATCAGGAGTAGGGATAAAATGCT
TGATGGTCGGAAGAGGCATAAATTCCGTCAGCCAGTTTAGTCTGACCATCTCATCTGTAACATCATTGGGAACGCTACCT
TTGCCATGTTTCAGAAACAACTCTGGCGCATCGGGCTTCCCATACAATCGATAGATTGTCGCACCTGATTGCCCGACATT
ATCGCGAGCCCATTTATACCCATATAAATCAGCATCCATGTTGGAATTTAATCGCGGCCTCGAGCAAGACGTTTCCCGTT
GAATATGGCTCATAACACCCCTTGTATTACTGTTTATGTAAGCAGACAGTTTTATTGTTCATGATGATATATTTTTATCT
TGTGCAATGTAACATCAGAGATTTTGAGACACAACGTGGCTTTCCCCCCCCCCCCTGCAGGTCGACGGATCCGGGGAATT
CGTAATCATGGTCATAGCTGTTTCCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGGCGGTTTGCGTATTGGGCGCTCTTCCGCTT
CCTCGCTCACTGACTCGCTGCGCTCGGTCGTTCGGCTGCGGCGAGCGGTATCAGCTCACTCAAAGgcggtaatacggtta
tccacagaatcaggggataacgcagg

For both assembly methods, a base substitution for mismatch was introduced in the Nrul recognition sequence within the overlap sequence, such that the presence or absence of mismatch-derived fragments could be detected by Nrul cleavage.

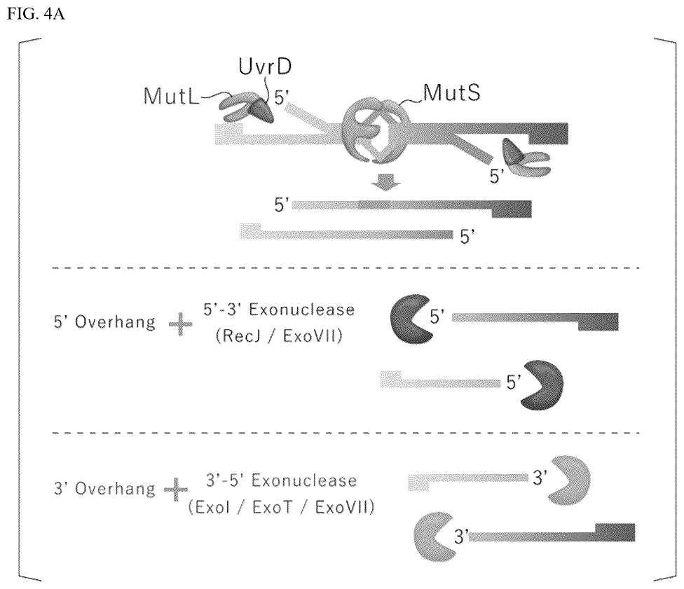

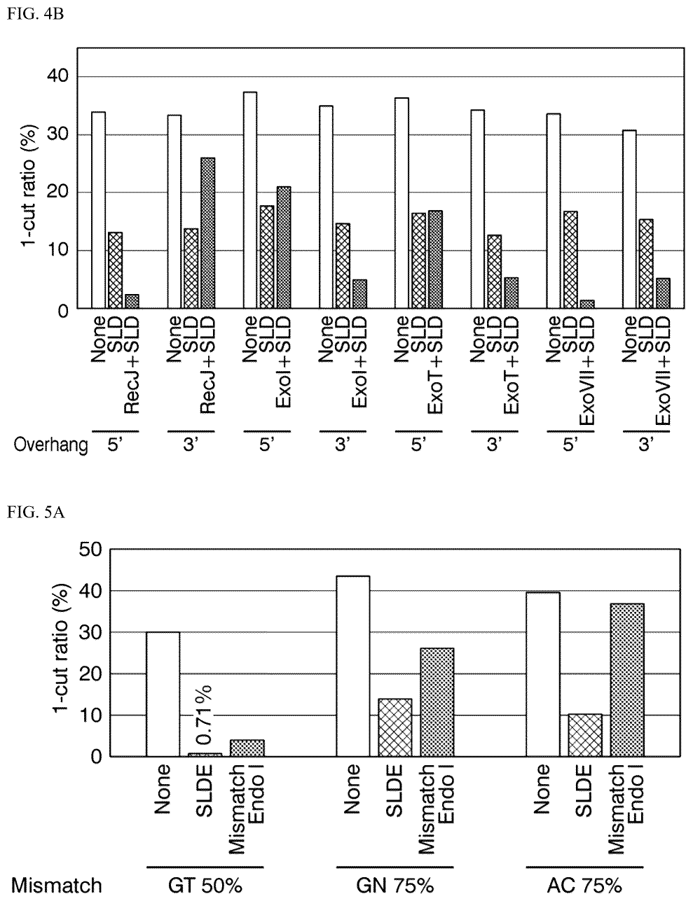

1 μL of each DNA assembly product was added to the reaction buffer in Table 2 containing the mismatch repair-related enzyme group SLD (300 nM MutS, 300 nM MutL, and 15 nM UvrD) and one single-strand specific exonuclease (3 U/μL RecJ, 100 mU/μL ExoI, 500 mU/μL ExoT or 100 mU/μL ExoVII) (5 μL total) and kept at 37° C. for 30 minutes to remove mismatches before DNA amplification. The same reaction was performed without adding enzymes as a control (None). The samples after the mismatch removal reaction were subjected to the same RCR amplification reaction as in Example 1, and the amplified products were cleaved with the restriction enzyme Nrul and analyzed by agarose gel electrophoresis. As in Example 1, the ratio of the mismatch-derived fragment (4.7 kb) that remained uncut from the circular DNA with the mismatch-derived base substitution was quantified as a percentage of all band intensities (1-cut ratio). The results are shown in FIG. 4B.

In the case of RA assembly, in which the 5′ overhang strands hybridize with each other, single-strand-specific exonucleases in the 5′-3′ direction was found to increase the mismatch removal effect. . . . In the case of USER (registered trademark) Cloning, in which the 3′ overhang strand hybridizes, a single-strand-specific exonuclease in the 3′-5′ direction was found to increase the mismatch removal effect. In addition, ExoVII, which has single-strand-specific exonuclease activity in both directions, increased the mismatch removal effect for the assembly product, regardless of the assembly method.

A schematic diagram of each assembly reaction and the action of single-strand-specific exonucleases is shown in FIG. 4A. Although not bound by theory, in the case of 5′ overhangs using the RA assembly reaction, the action of RecJ or ExoVII, which degrades nucleotides from the 5′ end, removes the overhang and suppresses cyclization by re-assembly, and amplification of DNA with sequence errors is more suppressed. In this way, the mismatch removal effect of SLD, a mismatch repair-related enzyme group, is thought to be increased. Conversely, in the case of 3′ overhangs using USER (registered trademark) Cloning, the action of ExoI, ExoT, or ExoVII, which degrades nucleotides from the 3′ end, removes the overhang, and the mismatch removal effect of a mismatch repair-related enzyme group SLD, is thought to be increased.

### [Example 5] Comparison with Various Conventional Mismatch-Cleaving Enzymes

Mismatch removal reactions were performed using mismatch endonuclease, which recognizes mismatches and performs DNA double-strand breaks, and the effects were compared with those of mismatch removal reactions using a mismatch repair-related enzyme group SLDE.

The three fragment groups shown in Table 14 below were used to obtain circular double-stranded DNA by the RA assembly reaction as in Example 1 or by USER (registered trademark) Cloning (NEB) as in Example 4. The sequences shown in Table 15 were used for introducing sequence errors by the RA assembly reaction. For introducing sequence errors by USER (registered trademark) Cloning (NEB), PCR fragments prepared using the templates and primers shown in Tables 11 and 16 were used. The mismatch removal reaction before DNA amplification and RCR amplification reaction were then performed as in Example 1, and the percentage of bands of Nrul uncut mismatch-derived fragments (1-cut ratio) was quantified and graphed. The mismatch removal enzymes used were the mismatch repair-related enzyme group SLDE (300 nM MutS, 300 nM MutL, 15 nM UvrD and 20 mU/μL ExoVII) or 8 U/μL Mismatch Endonuclease I (NEB).

TABLE 14

| | |
|---|---|
| GT 50% | 6 nM UPL fragment (2.8 kb)(SEQ ID NO. 20) |
| RA assembly | 3 nM UPR_GT fragment (2.0 kb)(Table 15) |
| reaction | 3 nM UPR fragment (2.0 kb)(SEQ ID NO. 21) |
| GN 75% | 6 nM UPL\|fragment (2.8 kb)(Table 20) |
| RA: assembly | 6 nM UPR_GN\|fragment (2.0 kb)(Table 15) |
| reaction | |
| AC 75% | 6 nM UPL-U fragment (2.8 kb)(Table 11) |
| USER | 4.5 nM UPR-U_AC fragment (2.0 kb)(Table 16) |
| Cloning | 1.5 nM UPR-U\|fragment (2.0 kb)(Table 11) |

TABLE 15

| Base substitutions introduced into the overlapping sequence at 5′ end of | | |
|---|---|---|
| UPR fragment (Underline: NruI recognition sequence of its modified sequence. | | SEQ |
| Uppercase: Base substitution. N: base mixture of A, G, C and T) | | ID NO. |
| No mismatch | aacagccctgctgcgcggttcgcgacataatgttcccgat | 22 |
| (UPR) | | |
| GN mismatch | aacagccctgctgcgcggttNgcgacataatgttcccgat | 23 |
| (UPR_GN) | | |
| G T mismatch | aacagccctgctgcgcggttTgcgacataatgttcccgat | 29 |
| (UPR_GT) | | |

TABLE 16

| Template and primer pair used for PCR amplification of UPR-U_AC fragment with | | | |
|---|---|---|---|
| base substitutions (Underline: NruI recognition sequence of its modified | | | SEQ |
| sequence. Uppercase: Base substitution and dU) | | | ID NO. |
| UPR- | template | UPR fragment | 21 |
| U_AC | primer | aacagcccUgcagcgcggUtTgcgacaUaatgttcccgaUtc | 30 |
| fragment | | atgttctttccUgcgttatccccUgattctgtggataaccgUattac | 25 |

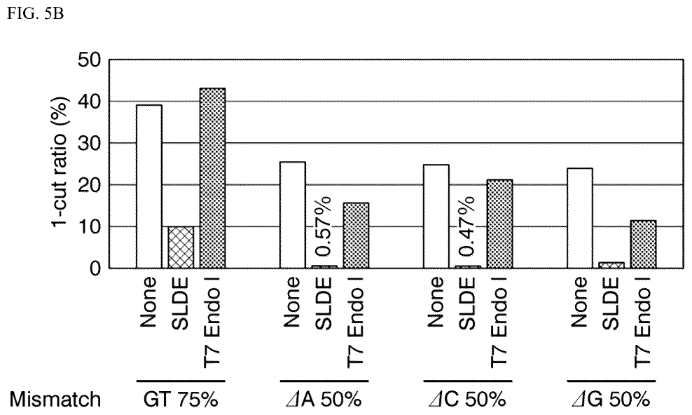

The results are shown in FIG. 5A. For all mismatch pairs examined, higher mismatch removal was achieved with the mismatch repair-related enzyme group SLDE.

Next, we used T7 endonuclease I (NEB), which has mismatch DNA cleavage activity and is also used to remove gene synthesis errors, in the same way and compared it to the mismatch repair-related enzyme group SLDE.

Circular DNA was obtained by RA assembly reaction as in Example 1, using the four fragment groups shown in Table 17 below. The sequences shown in Table 18 were used to introduce sequence errors. Mismatch removal and RCR amplification reactions were then performed as in Example 1, and the percentage of DNA bands of Nrul uncut mismatch-derived fragments (1-cut ratio) was quantified and graphed. The mismatch removal enzymes were the mismatch repair-related enzyme group SLDE (300 nM MutS, 300 nM MutL, 15 nM UvrD and 20 mU/μL ExoVII) or 0.1 U/μL T7 Endonuclease I (NEB).

TABLE 17

| | |
|---|---|
| GT 75% | 6 nM UPL\|fragment (2.8 kb)(SEQ ID NO. 20) |
| | 4.5 nM UPR\|fragment (2.0 kb)(SEQ ID NO. 21) |
| | 1.5 nM UPR_GT\|fragment (2.0 kb)(SEQ ID NO. 29) |
| ΔA 50% | 6 nM UPL\|fragment (2.8 kb)(SEQ ID NO. 20) |
| | 3 nM UPR\|fragment (2.0 kb)(SEQ ID NO. 21) |
| | 3 nM UPR_ΔA fragment (2.0 kb)(SEQ ID NO. 33) |
| ΔC 50% | 6 nM UPL fragment (2.8 kb)(SEQ ID NO. 20) |
| | 3 nM UPR\|fragment (2.0 kb)(SEQ ID NO. 21) |
| | 3 nM UPR_ΔC\|fragment (2.0 kb)(SEQ ID NO. 34) |
| ΔG 50% | 6 nM UPL fragment (2.8 kb)(SEQ ID NO. 20) |
| | 3 nM UPR fragment (2.0 kb)(SEQ ID NO. 21) |
| | 3 nM UPR_ΔG\|fragment (2.0 kb)(SEQ ID NO. 35) |

TABLE 18

| Base substitutions introduced into the overlapping sequence at 5′ | | |
|---|---|---|
| end of UPR fragment (Underline: NruI recognition sequence of its | | SEQ |
| modified sequence. Uppercase: Base substitution) | | ID NO. |
| No mismatch | aacagccctgctgcgcggttcgcgacataatgttcccgat | 22 |
| (UPR) | | |
| GT mismatch | aacagccctgctgcgcggttTgcgacataatgttcccgat | 29 |
| (UPR_GT) | | |
| A: deletion | aacagccctgctgcgcggttcgcg-cataatgttcccgat | 33 |
| (UPR_ΔA) | | |
| C: deletion | aacagccctgctgcgcggttcg-gacataatgttcccgat | 34 |
| (UPR_ΔC) | | |
| G: deletion | aacagccctgctgcgcggttc-cgacataatgttcccgat | 35 |
| (UPR_ΔG) | | |

The results are shown in FIG. 5B. For all single nucleotide deletion and GT mismatch pairs examined, higher sequence error removal was achieved with the mismatch repair-related enzyme group SLDE.

### [Example 6] NGS Analysis of Sequence Error Removal Effect

NGS was used to detect the effect of sequence error removal by a mismatch repair-related enzyme group. The adapter sequences necessary for NGS analysis were designed in advance in circular DNA, and sequence fragments were obtained by restriction enzyme BsaI cleavage without PCR amplification to avoid introducing errors during sample preparation. Furthermore, in paired-end analysis, reads containing mismatched bases between pairs were excluded from the analysis as sequence errors, so that low-frequency sequence errors in synthetic oligo DNA could be accurately analyzed. Since low-frequency sequence errors in synthetic oligo DNA are probabilistically mismatched when complementary oligo DNAs are hybridized to each other, we expected that DNA molecules with sequence errors could be removed by the mismatch removal reaction.

Two oligo DNAs (Oligo 1 and Oligo 2; Eurofins Oligonucleotide Purification Cartridge purification grade) with complementary sequences except for one end of each were hybridized to prepare oligo double-stranded DNA with overhangs at both ends (Oligo dsDNA). Oligo 1_GT, in which the 93rd base C of oligo 1 was replaced with T, was used as oligo DNA with an artificial error, and hybridized with oligo 2 in the same manner to prepare oligo double-stranded DNA with GT mismatch (Oligo dsDNA_GT). These were assembled to a 4.6 kb pUPiSeq fragment containing dU at the homologous end by USER (registered trademark) Cloning method to obtain circular DNA.

TABLE 19

| Uppercase: Complementary sequence between oligo DNAs. Lowercase: overlapping | | SEQ |
|---|---|---|
| sequence with pUPiSeq fragment. Underline: Base substitution | | ID NO. |
| Oligo 1 | TTTCGGGATTAAAAAACTGAAAAAAGGGGGGGGCAGTTCCTGGTTACGTGACCTGA | 36 |
| | TTTACTGGTATATGCCAACAGCCCTGCTGCGCGGTTCGCGACATAATGTTCCCGAT | |
| | TCgtgtttccggcagtggattaaataaaactgct | |
| Oligo 1_GT | TTTCGGGATTAAAAAACTGAAAAAAGGGGGGGGCAGTTCCTGGTTACGTGACCTGA | 37 |
| | TTTACTGGTATATGCCAACAGCCCTGCTGCGCGGTTTGCGACATAATGTTCCCGAT | |
| | TCgtgtttccggcagtggattaaataaaactgct | |
| Oligo 2 | GAATCGGGAAGATTATGTCGCGAACCGCGCAGCAGGGCTGTTGGCATATACCAGTA | 38 |
| | AATCAGGTCACGTAACCAGGAACTGCCCCGCCCTTTTTTCAGTTTTTTAATCCCGA | |
| | AAtaaaccagaaccgctgcaccaataccgaacact | |

The pUPiSeq fragment (4.6 kb) was prepared by PCR amplification of a plasmid obtained by assembly and cyclization of UPL fragment 2.8 kb (SEQ ID NO. 20) and UPR fragment 2.0 kb (SEQ ID NO. 21) by the same method as in Example 5, using primer 1 and primer 2 (USER (registered trademark) primers for cloning) containing dU.

TABLE 20

| Uppercase: dU | | SEQ ID NO. |
|---|---|---|
| Primer 1 | gtgttUccggcagtggaUtaaataaaactgcUg | 39 |
| Primer 2 | taaaccagaaccgcUgcaccaaUaccgaacacUg | 40 |

Specifically, 3.2 nM Oligo dsDNA, 3.2 nM Oligo dsDNA_GT, and 1.6 nM pUPiSeq fragment were added to CutSmart buffer (NEB) containing 20 mU/μL of Thermolabile USER II Enzyme (NEB) (5 μL total), followed by uracil removal reaction at 37° C. for 30 min, followed by heat treatment at 75° C. for 5 min and slow cooling to obtain circular DNA assembly products.

1 μL of the resulting circular DNA assembly product was subjected to the mismatch removal reaction prior to DNA amplification as in Example 1 (5 μL total), and further warmed at 37° C. for 30 minutes. After warming, 2.5 μL of the sample was added to the RCR amplification reaction solution (5 μL total), and the RCR amplification reaction at 30° C. for 16 hours and finalization treatment were performed as in Example 1.

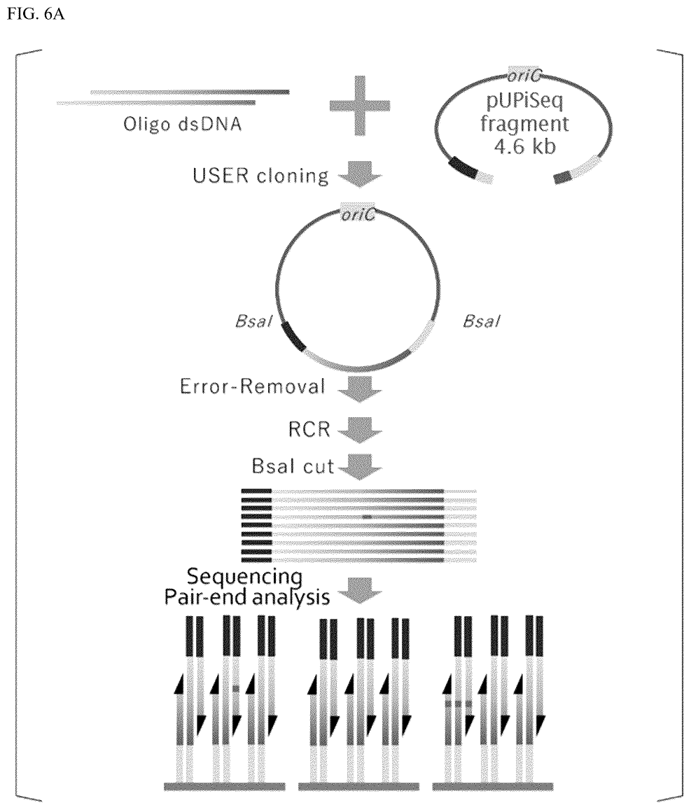

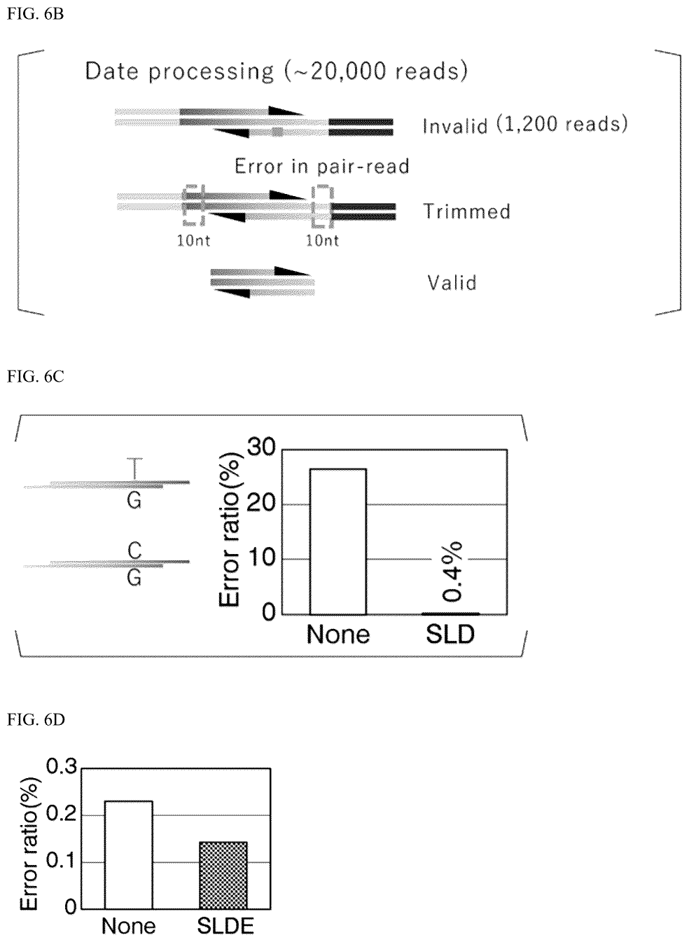

The resulting RCR amplification product was cut with the restriction enzyme BsaI to obtain a 311 base pair DNA fragment with Illumina sequencing adapter sequences at both ends. This fragment was subjected to paired-end analysis using the Illumina iSeq (registered trademark) 100 sequencing system, and approximately 200,000 valid reads were obtained. Approximately 1,200 reads that were mismatches between paired reads were excluded from the reads obtained by the paired-end analysis, and the valid reads were analyzed by excluding the 10 bases at both ends of the reads. The percentage of bases that differ from the designed bases (including substitutions introduced as mismatches) among all analyzed bases is plotted as the error ratio. A schematic diagram of this experiment is shown in FIG. 6A, a schematic diagram of data processing in the experiment in FIG. 6B, and the results in FIGS. 6C and 6D.

FIG. 6C shows the results of mismatch removal reactions and RCR amplification and NGS analysis, using samples containing 50% of circular DNA assembly products with GT mismatches in a reaction system with (SLD) or without (None) the mismatch repair-related enzyme group SLD (300 nM MutS, 300 nM MutL, and 15 nM UvrD). In the amplification of a mixture of circular DNA with artificially introduced mismatches and circular DNA without mismatches, the use of a mismatch repair-related enzyme group SLD, resulted in significantly higher mismatch removal, which was also confirmed by NGS.

FIG. 6D shows the results of mismatch removal reactions with (SLDE) or without (None) the mismatch repair-related enzyme group SLDE (300 nM MutS, 300 nM MutL, 15 nM UvrD, 20 mU/μL ExoVII) using only circular DNA assembly products without artificial mismatch. The results of RCR amplification and NGS analysis are shown in the same manner as in FIG. 6C. In the amplification without mismatch repair-related enzymes, an error of 0.23% was detected, which was thought to be due to an oligo DNA synthesis error, but this error rate was suppressed to 0.12% by the mismatch removal reaction with the mismatch repair-related enzyme group SLDE. Not only artificially introduced sequence errors, but also low-frequency sequence errors that occur during oligo DNA synthesis were also removed by the mismatch repair-related enzyme group.

### [Example 7] Effect of Sequence Error Removal on Artificial Genes Synthesized from Oligo DNA-1

Artificial genes were synthesized by hybridizing multiple oligo DNAs, and the effect of removing sequence errors inherent in the genes was examined by detecting gene function as an indicator. The basic reactions of DNA assembly and cyclization, mismatch removal, and RCR amplification were performed using the same techniques as in Example 6, unless otherwise stated.

First, 200 base of lacZ gene 5′ end (5′-lacZ) was prepared by hybridizing two types of oligo DNA (oligo 3 and oligo 4; Eurofins Oligonucleotide Purification Cartridge purification grade) with a single strand that serves as a homologous region with the pKOZ fragment at the end. The 5′-lacZ was prepared as an artificial gene. This 5′-lacZ fragment (6.9 nM) was mixed with a 0.9 nM pPKOZ fragment (8.7 kb) containing dU at the homologous end, and assembly and cyclization was performed by USER (registered trademark) Cloning method.

TABLE 21

| Uppercase: Complementary sequence between oligo DNAs | | SEQ |
|---|---|---|
| Lowercase: overlapping sequence with pPKOZ fragment | | ID NO. |
| Oligo 3 | GGGAAAACCCTGGCGTTACCCAACTTAATCGCCTTGCAGCACATCCCCCTTTCGCCA | 41 |
| | GCTGGCGTAATAGCGAAGAGGCCCGCACCGATCGCCCTTCCCAACAGTTGCGCAGCC | |
| | TGAATGgcgaatggcgctttgcctggtttccggcaccagaagcggt | |
| Oligo 4 | CATTCAGGCTGCGCAACTGTTGGGAAGGGCGATCGGTGCGGGCCTCTTCGCTATTAC | 42 |
| | GCCAGCTGGCGAAAGGGGGATGTGCTGCAAGGCGATTAAGTTGGGTAACGCCAGGGT | |
| | TTTCCCagtcacgacgttgtaaaacgacggccagtgaatccgtaat | |

The pPKOZ fragment (8.7 kb) was prepared using plasmid pPKOZ (Su'etsugu et al., Nucleic Acids Research, 2017, vol. 45, p. 45, 20, p.11525-11534), with lacZ gene, encoding oriC, kanamycin resistance gene, and β-galactosidase) as a template, and by PCR amplification with primers 3 and 4 containing dU.

TABLE 22

| Uppercase: dU | | SEQ ID NO. |
|---|---|---|
| Primer 3 | gcgaaUggcgcttUgcctggttUccggcaccagaagcggUg | 43 |
| Primer 4 | agtcacgacgttgUaaaacgacggccagUgaatccgtaaUc | 44 |

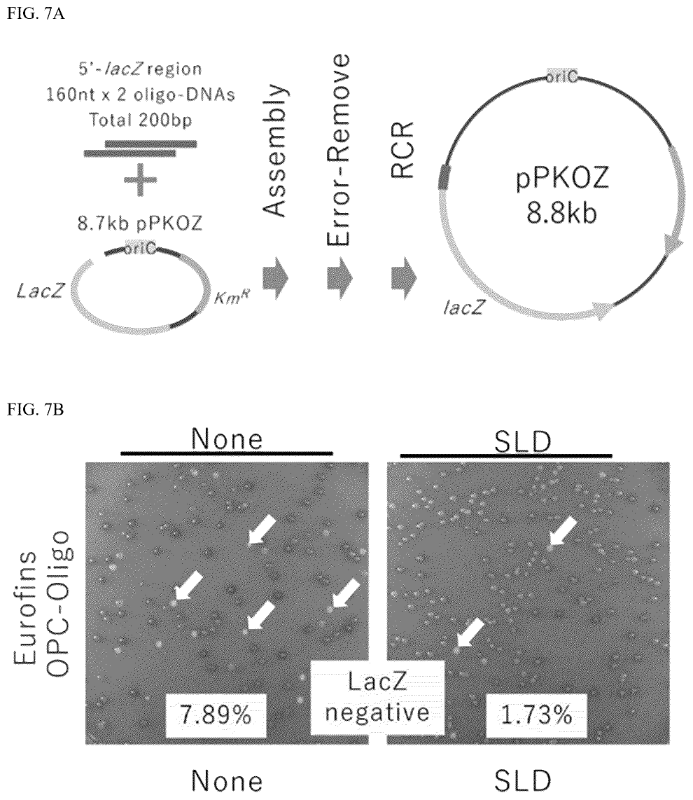

1 μL of the reaction solution after assembly and cyclization was subjected to a mismatch removal reaction in a reaction solution with (SLD) or without (None) the mismatch repair-related enzyme group SLD (300 nM MutS, 300 nM MutL, 15 nM UvrD) (5 μL total) at 37° C. for 60 minutes, followed by the RCR amplification reaction. FIG. 7A shows a schematic diagram of the experiment.

Subsequently, 1 μL of RCR amplification product was used to transform E. coli DH5α strain (Takara Bio). The transformed E. coli was inoculated on LB plates containing 25 μg/mL kanamycin, 0.1 mM IPTG, and 20 ng/μL X-Gal, and incubated at 37° C. overnight. E. coli colonies transformed with DNA carrying the wild-type lacZ gene represent blue, while E. coli colonies transformed with DNA carrying the lacZ gene mutation represent white. The number of colonies was counted and the ratio of white colonies to the total number of colonies was expressed as LacZ negative. The results are shown in FIG. 7B.

As shown in FIG. 7B, 7.89% of white colonies (an example is indicated by an arrow in FIG. 7B) were detected without the addition of the mismatch repair-related enzyme group SLD. This was thought to be due to a lacZ gene mutation resulting from an oligo DNA synthesis error. On the other hand, when the mismatch repair-related enzyme group SLD was used, the percentage of white colonies was suppressed to 1.73%, confirming the effect of removing sequence errors that occur during oligo DNA synthesis.

### [Example 8] Effect of Sequence Error Removal on Artificial Genes Synthesized from Oligo DNA-2

Sixteen different oligo DNA sequences of about 100 bases each were designed so that about 800 base pairs of artificial genes, including the gfp gene, would be generated by hybridization of oligo DNA with each other as double-stranded DNA containing no single-stranded gaps. Table 23 shows the design of Eurofins PAGE purification grade oligo DNA (Eurofins PAGE-Oligo, Eurofins Oligonucleotide Purification Cartridge purification grade). Table 24 is the design of IDT's oPools oligo pools (IDT oPools). Mismatch removal and RCR amplification reactions were performed with these oligo DNAs using the same methods as in Example 6, as described below.

TABLE 23
Oligo DNA (Uppercase: Complementary sequence between oligo DNAs. Lowercase:
overlapping sequence with pUPiSeq fragment

| | |
|---|---|
| Oligo E1 | CAACAAGAATTGGGACAACTCCAGTGAAAAGTTCTTCTCCTTTGCTCATggtatatctccttcttaagttaaacaaaatt |
| | atttctagaggatgcgcat (SEQ ID NO 45) |
| Oligo E2 | ATGAGCAAAGGAGAAGAACTTTTCACTGGAGTTGTCCCAATTCTTGTTGAATTAGATGGTGATGTTAATGGGCACAAATT |
| | TTCTGTCCGTGGAGAGGGT (SEQ ID NO 46) |
| Oligo E3 | GTAGTGCAAATAAATTTAAGGGTGAGTTTTCCGTTTGTAGCATGAGCTTCACCCTCTCCACGGACAGAAAATTTGTGCCG |
| | ATTAACATCACCATCTAATT (SEQ ID NO 47) |
| Oligo E4 | GAAGGTGATGCTACAAACGGAAAACTCACCCTTAAATTTATTTGCACTACTGGAAAACTACCTGTTCCGTGGGGAACACT |
| | TGTCACTACTCT (SEQ ID NO 48) |
| Oligo E5 | GTTTCATGTGATCCGGATAACGGGAAAAGCATTGAACACCATAGGTCAGAGTAGTGACAAGTGTTGGCCACGGAACAGGT |
| | AGTTTTCCA (SEQ ID NO 49) |
| Oligo E6 | GACCTATGGTGTTCAATGCTTTTCCCGTTATCCGGATCACATGAAACGGCATGACTTTTTCAAGAGTGCCATGCCCGAAG |
| | GTTATGTACA (SEQ ID NO 50) |
| Oligo E7 | CGTCTTGTAGGTCCCGTCATCTTTGAAAGATATAGTGCGTTCCTGTACATAACCTTCGGGGATGGCACTCTTGAAAAAGT |
| | CATGCC (SEQ ID NO 51) |
| Oligo E8 | GGAACGCACTATATCTTTCAAAGATGACGGGACCTACAAGACGCGTGCTGAAGTGAAGTTTGAAGGTGATACCCTTGTTA |
| | ATCGTATCGAGTT (SEQ ID NO 52) |
| Oligo E9 | CAAGTTTGTGTCCAAGAATGTTTCCATCTTCTTTAAAATCAATACCCTTTAACTCGATACGATTAACAAGGGTATCACCT |
| | TCAAACTTGACTTCAGCACG (SEQ ID NO 53) |
| Oligo E10 | AAAGGGTATTGATTTTAAAGAAGATGGAAACATTCTTGGACACAAACTTGAGTACAACTTTAACTCACACAATGTATACA |
| | TGAGGGGAGACAAACAAAAG (SEQ ID NO 54) |
| Oligo E11 | GAACCATCTTCAACGTTGTGGCGAATTTTGAAGTTAGCTTTGATTGGATTCTTTTGTTTGTGTGCCGTGATGTATACATT |
| | GTGTGAGTTAAAGTTGTACT (SEQ ID NO 55) |
| Oligo E12 | AATGGAATCAAAGCTAACTTCAAAATTCGCCACAACGTTGAAGATGGTTCCGTTCAACTAGCAGACCATTATCAACAAAA |
| | TACTCCAATTGGGGATGGCC (SEQ ID NO 56) |
| Oligo E13 | AAAGGACAGATTGTGTCGACAGGTAATGGTTGTCTGGTAAAAGGACAGGGCCATCGCCAATTGGAGTATTTTGTTGATAA |
| | TGGTCTGCTAGTTGAAGG (SEQ ID NO 57) |
| Oligo E14 | CTGTCCTTTTACCAGACAACCATTACCTGTCGACACAATCTGTCCTTTCGAAAGATCCCAACGAAAAGCGTGACCACATG |
| | GTCCTTCTT (SEQ ID NO 58) |
| Oligo E15 | ttaATACAGCTCATCCATACCATGTGTAATCCCAGCAGCAGTTACAAACTCAAGAAGGACCATGTGGTCACGCTTTTCGT |
| | TGGGATCTTTCG (SEQ ID NO 59) |
| Oligo E16 | GAGTTTGTAACTGCTGCTGGGATTACACATGGTATGGATGAGCTGTATtaaatggactcgtctactagcgcagcttaatt |
| | aacctaggctgct (SEQ ID NO 60) |

TABLE 24
Oligo pool (Uppercase: complementary sequence between oligo DNAs. Lowercase:
overlapping sequence with pUPiSeq fragment

| | |
|---|---|
| Oligo P1 | AAGAATTGGGACAACTCCAGTGAAAAGTTCTTCTCCTTTGCTCATggtatatctccttcttaagttaaacaaaattattt |
| | ctagaggatgcgcat (SEQ ID NO 61) |
| Oligo P2 | ATGAGCAAAGGAGAAGAACTTTTCACTGGAGTTGTCCCAATTCTTGTTGAATTAGATGGTGATGTTAATGGGCACAAATT |
| | TTCTGTCCGTGGAGA (SEQ ID NO 62) |
| Oligo P3 | AATAAATTTAAGGGTGAGTTTTCCGTTTGTAGCATCACCTTCACCCTCTCCACGGACAGAAAATTTGTGCCCATTAACAT |
| | CACCATCTAATTCAAC (SEQ ID NO 63) |
| Oligo P4 | GGGTGAAGGTGATGCTACAAACGGAAAACTCACCCTTAAATTTATTTGCACTACTGGAAAACTACCTGTTCCGTGGCCAA |
| | CACTTGTCACTACTC (SEQ ID NO 64) |
| Oligo P5 | TTCATGTGATCCGGATAACGGGAAAAGCATTGAACACCATAGGTCAGAGTAGTGACAAGTGTTGGCCACGGAACAGGTAG |
| | TTTTCCAGTAGTGCA (SEQ ID NO 65) |
| Oligo P6 | TGACCTATGGTGTTCAATGCTTTTCCCGTTATCCGGATCACATGAAACGGCATGACTTTTTCAAGAGTGCCATGCCCGAA |
| | GGTTATGTACAGGAA (SEQ ID NO 66) |
| Oligo P7 | CAGCACGCGTCTTGTAGGTCCCGTCATCTTTGAAAGATATAGTGCGTTCCTGTACATAACCTTCGGGCATGGCACTCTTG |
| | AAAAAGTCATGCCGT (SEQ ID NO 67) |
| Oligo P8 | CGCACTATATCTTTCAAAGATGACGGGACCTACAAGACGCGTGCTGAAGTCAAGTTTGAAGGTGATACCCTTGTTAATCG |
| | TATCGAGTTAAAGGGT (SEQ ID NO 68) |
| Oligo P9 | ACTCAAGTTTGTGTCCAAGAATGTTTCCATCTTCTTTAAAATCAATACCCTTTAACTCGATACGATTAACAAGGGTATCA |
| | CCTTCAAACTTGACTT (SEQ ID NO 69) |
| Oligo P10 | ATTGATTTTAAAGAAGATGGAAACATTCTTGGACACAAACTTGAGTACAACTTTAACTCACACAATGTATACATCACGGC |
| | AGACAAACAAAAGAAT (SEQ ID NO 70) |
| Oligo P11 | AACCATCTTCAACGTTGTGGCGAATTTTGAAGTTAGCTTTGATTCCATTCTTTTGTTTGTCTGCCGTGATGTATACATTG |
| | TGTGAGTTAAAGTTGT (SEQ ID NO 71) |
| Oligo P12 | GGAATCAAAGCTAACTTCAAAATTCGCGACAACGTTGAAGATGGTTCCGTTCAACTAGCAGACCATTATCAACAAAATAC |
| | TCCAATTGGCGATGGC (SEQ ID NO 72) |
| Oligo P13 | GACAGATTGTGTCGACAGGTAATGGTTGTCTGGTAAAAGGACAGGGGCATCGCCAATTGGAGTATTTTGTTGATAATGGT |
| | CTGCTAGTTGAACGG (SEQ ID NO 73) |
| Oligo P14 | CCTGTCCTTTTACCAGACAACCATTACCTGTCGACACAATCTGTCCTTTCGAAAGATCCCAACGAAAAGCGTGACCACAT |
| | GGTCCTTCTTGAGTT (SEQ ID NO 74) |
| Oligo P15 | ttaATACAGCTCATCCATACCATGTGTAATCCCAGCAGCAGTTACAAACTCAAGAAGGACCATGTGGTCACGCTTTTCGT |
| | TGGGATGTTTCGAAAG (SEQ ID NO 75) |
| Oligo P16 | TGTAACTGCTGCIGGGATTACAGATGGTATGGATGAGCTGTATtaaatggactcgtctactagcgcagcttaattaacct |
| | aggctgctgccaccgct (SEQ ID NO 76) |

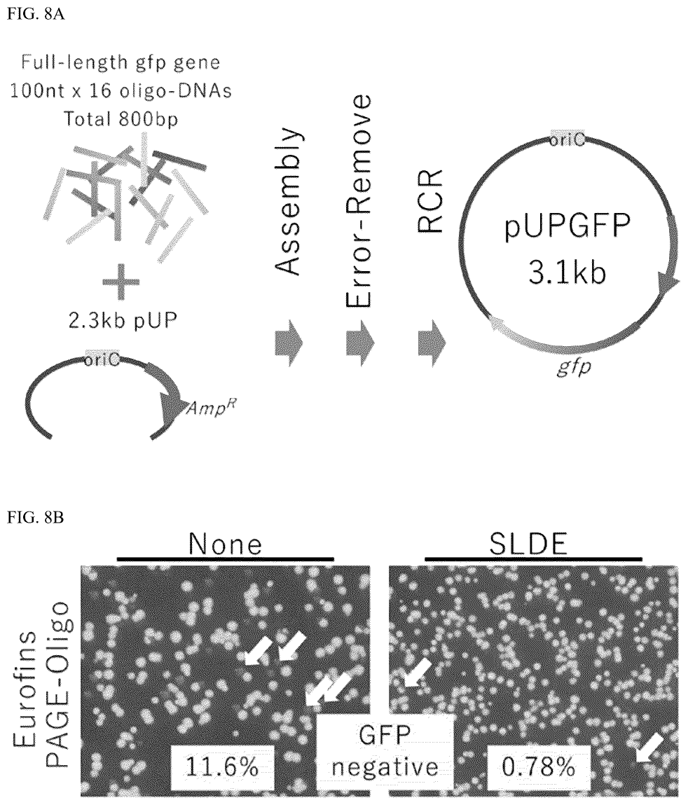

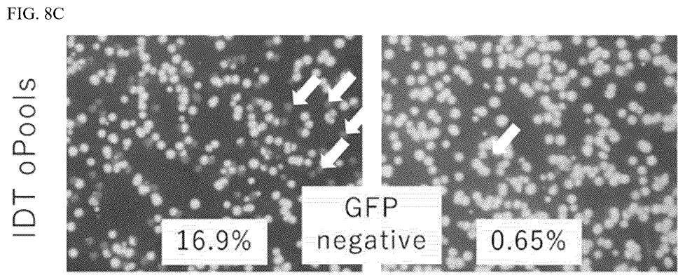

For pUP-1 fragment (2.3 kb) and 16 Eurofins PAGE-Oligo, the respective concentrations were equalized and added to buffer containing 10 mU/μL Thermolabile USER II Enzyme (NEB) (20 mM Tris-HCl pH 8.0, 20 mM Mg (oAc)₂, 50 mM potassium glutamate, 150 mM tetramethylammonium chloride, 4 mM dithiothreitol (DTT), 5% glycerol, 5% PEG8000, 100 μM ATP, 4 mM creatine phosphate, 20 ng/μL creatine kinase, and 0.7 μM RecA). Next, after uracil removal reaction at 37° C. for 15 min, the oligo DNA and single-stranded overhangs were assembled and cyclized by heat treatment at 75° C. for 5 min and slow cooling (0.1° C./second). For 1 μL of the resulting reaction solution, mismatch removal was performed with (SLDE) or without (None) the mismatch repair-related enzyme group SLDE (300 nM MutS, 300 nM MutL, 15 nM UvrD, 20 mU/μL ExoVII) (5 μL total) at 37° C. for 30 minutes, followed by RCR amplification reaction. FIG. 8A shows a schematic diagram of the experiment.

Subsequently, 1 μL of RCR amplification product was used to transform the E. coli DH5a strain. The transformed E. coli was inoculated on LB plates containing 100 μg/mL ampicillin and incubated at 37° C. overnight. E. coli colonies transformed with DNA carrying the wild-type gfp gene (pUPGFP) exhibited green fluorescence, while E. coli colonies transformed with DNA carrying a mutation in the gfp gene did not. The number of colonies that did not fluoresce was counted, and the ratio of the number of colonies that did not fluoresce to the total number of colonies was expressed as GFP negative.

Similar experiments were performed with the combination of pUP-2 fragment (2.3 kb) and the IDT oPools containing 16 different oligo DNAs.

The pUP-1 fragment (2.3 kb) was prepared by PCR amplification with primers 5 and 6 containing dU using the pUP fragment (SEQ ID NO. 31) with oriC, ampicillin resistance gene and pUCori as template. pUP-2 fragment (2.3 kb) was prepared by changing primer 5 to primer 7 in the same PCR amplification as pUP-1.

TABLE 25

| | | SEQ |
|---|---|---|
| Uppercase: dU | | ID NO. |
| Primer 5 | atggactcgtctacUagcgcagcttaatUaacctaggctgcUgc | 77 |
| Primer 6 | ggtatatctccttctUaagttaaacaaaattatttcUagaggatgcgcaUat | 78 |
| Primer 7 | atggactcgtctacUagcgcagcttaatUaacctaggctgcUgccaccgcUga | 32 |

TABLE 26
pUP fragment (2.3 kb) (SEQ ID NO. 31)
(Lower case: overlapping sequence with oligo DNAs)

atggactcgtctactagcgcagcttaattaacctaggctgctgccaccgctgaGCAATAAATAAAACGAAAGGCTCAGTC
GAAAGACTGGGCCTTTCGTTTTATCTGAAAGGAGGAACTATATCCGGATTGGCGCGCGGAACCCCTATTTGTTTATTTTT
CTAAATACATTCAAATATGTATCCGCTCATGAGACAATAACCCTGATAAATGCTTCAATAATATTGAAAAAGGAAGAGTA
TGAGTATTCAACATTTCCGTGTCGCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATCGAACTGGATCTCAACAGCGGTAAGAT
CCTTGAGAGTTTTCGCCCCGAAGAACGTTTTCCAATGATGAGCACTTTTAAAGTTCTGCTATGTGGCGCGGTATTATCCC
GTATTGACGCCGGGCAAGAGCAACTCGGTCGCCGCATACACTATTCTCAGAATGACTTGGTTGAGTACTCACCAGTCACA
GAAAAGCATCTTACGGATGGCATGACAGTAAGAGAATTATGCAGTGCTGCCATAACCATGAGTGATAACACTGCGGCCAA
CTTACTTCTGACAACGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTAGCAATGGCAACAACG
TTGCGCAAACTATTAACTGGCGAACTACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCATTGGTAACTGTCAGACCAAGTTTACTC
ATATATACTTTAGATTGATTTAAAACTTCATTTTTAATTTAAAAGGATCTAGGTGAAGATCCTTTTTGATAATCTCATGA
CCAAAATCCCTTAACGTGAGTTTTCGTTCCACTGAGCGTCAGACCCCGTAGAAAAGAGAGCTGTTGACAGAGGGTCATTT
TCACACTATAATGCAGTGAATCCCAAACAGTATGTTGTACCTAAAGGGATCCTGGGTATTAAAAAGAAGATCTATTTATT
TAGAGATCTGTTCTATTGTGATCTCTTATTAGGATCGCACTGCCCTGTGGATAACAAGGATCCGGCTTTTAAGATCAACA
ACCTGGAAAGGATCATTAACTGTGAATGATCGGTGATCCTGGACCGTATAAGCTGGGATCAGAATGAGGGGTTATACACA
ACTCAAAAACTGAACAACAGTTGTTCTTTGGATAACTACCGGTTGATCCAAGCTTCCTGACAGAGTTATCCACAGTAGAT
CGCACGATGTGTCAGCTCATTTCCTTTAGGTACAACATACTAGAATATTTGCCTACAGCCTCCTTTCGGAGGCTGTTTTT
TTATCAAAGGATCTTCTTGAGATCCTTTTTTTCTGCGCGTAATCTGCTGCTTGCAAACAAAAAAACCACCGCTACCAGCG
GTGGTTTGTTTGCCGGATCAAGAGCTACCAACTCTTTTTCCGAAGGTAACTGGCTTCAGCAGAGCGCAGATACCAAATAC
TGTCCTTCTAGTGTAGCCGTAGTTAGGCCACCACTTCAAGAACTCTGTAGCACCGCCTACATACCTCGCTGTGCTAATCC
TGTTACCAGTGGCTGCTGCCAGTGGCGATAAGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCGAACGACCTACACCGAACTGAGATACCTACA
GCGTGAGCATTGAGAAAGCGCCACGCTTCCCGAAGGGAGAAAGGCGGACAGGTATCCGGTAAGCGGCAGGGTCGGAAGAG
GAGAGCGCACGAGGGAGCTTCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAATTGACATCCCTATCAGTGATAGATATAatatgogo
atcctctagaaataattttgtttaacttaagaaggagatatacc

Results are shown in FIG. 8B and FIG. 8C. The percentage of GFP negatives without the addition of mismatch repair-related enzyme groups was 11.6% in Eurofins PAGE-Oligo (FIG. 8B) and 16.9% in IDT oPools (FIG. 8C). An example of a colony that did not fluoresce is shown by arrows in FIGS. 8B and 8C. These were thought to be due to gfp gene mutations resulting from oligo DNA synthesis errors, and the synthesis error rate was increased due to the large number of oligo DNAs assembled and the longer total length of the oligo DNAs compared to Example 7. By using the mismatch repair-related enzyme group SLDE, the percentage of GFP negatives was suppressed to 0.78% in Eurofins PAGE-Oligo (FIG. 8B, suppression rate 1/15) and to 0.65% in IDT oPools (FIG. 8C, suppression rate 1/26). The sequence errors that occur during oligo DNA synthesis were removed.

The sequence error removal effects of the existing Surveyor nuclease and T7 endonuclease I have been similarly measured using fluorescent colony assays: Surveyor nuclease suppresses the percentage of non-fluorescent colonies from the original 50% to 16% at maximum, with a suppression ratio of 1/3 (Non-Patent Literature 4). T7 endonuclease I suppresses the percentage of non-fluorescent colonies from the original 69% to a maximum of 11% with a suppression ratio of 1/6 (Non-Patent Literature 5). From the comparison of these literature values and the results of this experiment, it can be said that the method of this application using a mismatch repair-related enzyme group SLDE, is more effective in removing sequence errors than conventional methods using Surveyor nuclease or T7 endonuclease I.

### [Example 9] Effect of Mismatch Repair-Related Enzymes on Replication Errors During DNA Amplification Reactions

We examined whether DNA polymerase replication errors occurred during DNA amplification reactions could be removed by a mismatch repair-related enzyme group.

Plasmid pUPkmGFP (3.9 kb) was constructed by replacing the ampicillin resistance gene of pUPGFP constructed in Example 8 with a kanamycin resistance gene, which was purified from E. coli after transformation and DNA sequence confirmed.

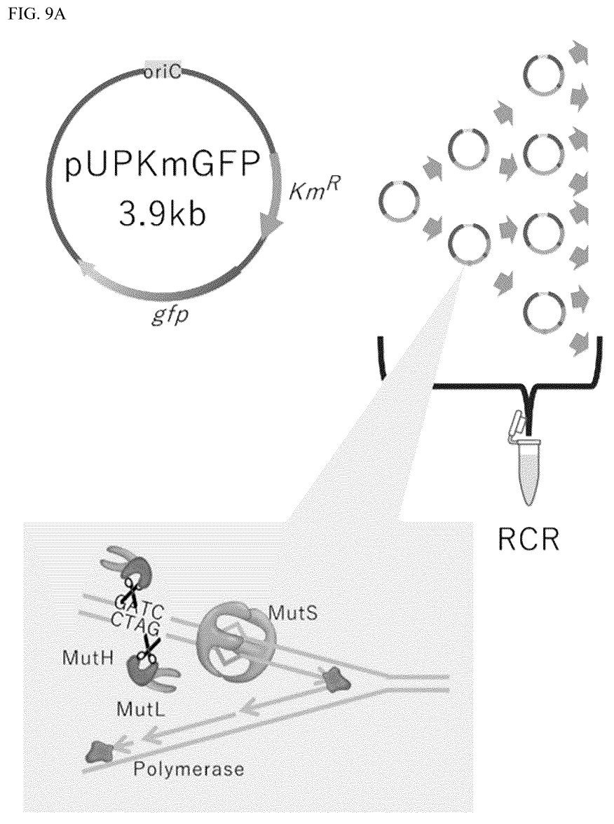

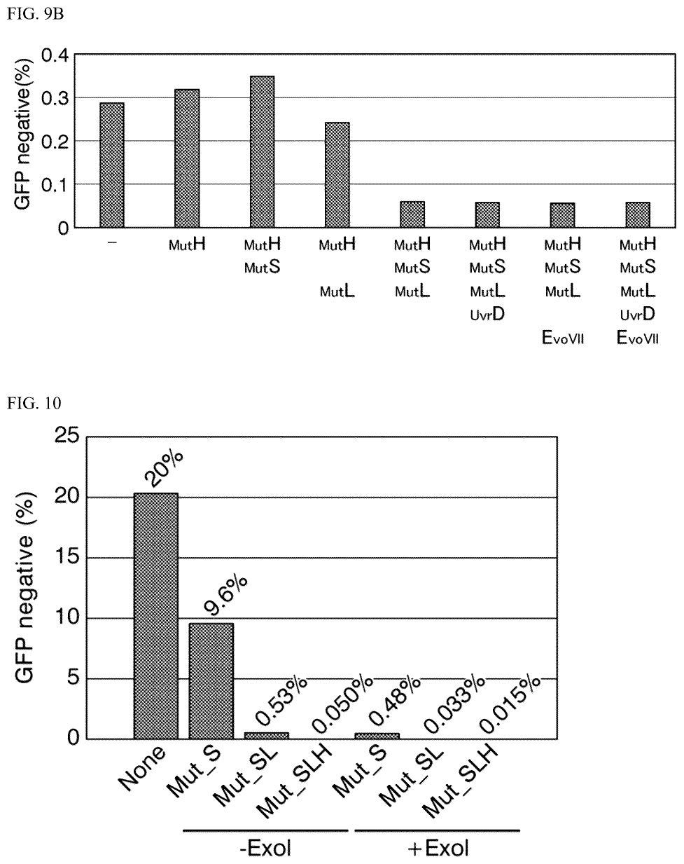

To the RCR amplification reaction solution, pUPkmGFP (final concentration 1 pM) was added (total 5 μL), and the RCR amplification reaction and finalization treatment were performed at 30° C. for 16 h as in Example 1. A mismatch repair-related enzyme group (100 nM MutH, 300 nM MutS, 300 nM MutL, 15 nM UvrD, and 20 mU/μL ExoVII, respectively) shown in FIG. 9B was also added to the reaction during RCR amplification.

For each reaction, 1 μL of RCR amplification product was used to transform E. coli DH5a strain by chemical method. The transformed E. coli was inoculated on LB plates containing 25 μg/mL kanamycin and incubated at 37° C. overnight. E. coli colonies transformed with DNA carrying the wild-type gfp gene exhibited green fluorescence, and the number of colonies that did not fluoresce due to the gfp gene mutation was counted and plotted as GFP negative as a percentage of the total number of colonies.

The results are shown in FIG. 9B: 0.28% GFP negatives were detected in the RCR amplified product, whereas the percentage of GFP negatives was suppressed to 0.06% in the product obtained by RCR amplification in the presence of MutS, MutL and MutH. Since no artificial sequence error was introduced in this example, it was confirmed that the addition of a mismatch repair-related enzyme group to the amplification reaction solution was effective in removing sequence errors, even for replication errors that occur during DNA amplification reactions. In Example 2, it was already confirmed that the addition of MutS and MutL as a mismatch repair-related enzyme group to the RCR amplification reaction solution had a sequence error removal effect, but the addition of MutS, MutL, and MutH as a of mismatch repair-related enzyme group to the RCR amplification reaction solution was thought to increase the sequence error removal effect. Although not bound by theory, a schematic diagram of this experiment and the action of the mismatch repair-related enzyme group is shown in FIG. 9A.

### [Example 10] Effect of a Mismatch Repair-Related Enzyme Group (Single-Strand Specific Exonuclease) During RCR Amplification Reaction

The effect of removing sequence errors was examined using about 800 artificial base pairs containing the gfp gene synthesized by oligo DNA hybridization as in Example 8 for a system in which mismatch removal reactions are performed simultaneously during DNA amplification reactions, as in Example 2. In doing so, the effect of adding ExoI, a single-strand specific exonuclease, in a mismatch repair-related enzyme group was also examined.

Assembly and cyclization of oligo DNA and single-stranded overhangs was performed using the pUP-2 fragment (2.3 kb) and IDT oPools containing 16 different oligo DNAs from Table 24, using the same technique as in Example 8. The resulting 0.5 μL of reaction solution was added to RCR reaction solution (5 μL) containing a mismatch repair-related enzyme group (150 nM MutS, 150 nM MutL, 150 nM MutH, 200 mU/μL ExoI) and amplification reaction was performed at 30° C. for 16 hours. ExoI is the same as that used in Example 1.

The RCR amplification product was then used to transform the E. coli DH5a strain, and the percentage of E. coli colonies that were GFP negative was counted using the same method as in Example 8. The counting results are shown in FIG. 10. In addition to the control without mismatch repair-related enzymes (None), the mismatch repair-related enzyme groups, MutS only (Mut_S), MutS and MutL (Mut_SL), and MutS, MutL, and MutH (Mut_SLH), each without (−Exo) or with (+Exo) ExoI, were examined.

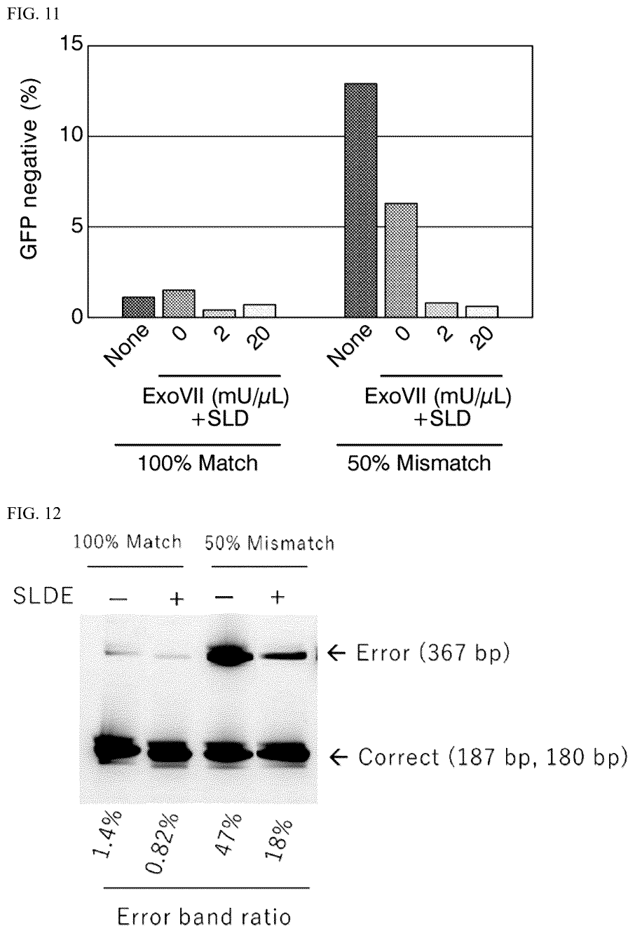

As shown in FIG. 11, the system in which a mismatch repair-related enzyme group was added during the DNA amplification reaction (1-step) showed a suppression of the percentage of GFP negative colonies, indicating that sequence errors in the gfp gene synthesized from oligo DNA could be removed. Although the addition of MutS alone during the amplification reaction was effective, its effect was enhanced by the addition of MutL and further enhanced by the addition of MutH. The error removing effect was further enhanced by the addition of ExoI in each experiment. The percentage of GFP negative colonies was suppressed to 0.015% by using RCR amplification reactions containing the four enzymes MutS, MutL, MutH, and ExoI. The error rate calculated from this percentage was 1 error per 1.6×10⁶ bases. The error rate was calculated using the following formula, based on the assumption that 1/3 of the gfp gene sequence, or 234 bases, is the site that produces GFP negative colonies. In the formula, F is the percentage of GFP negative colonies.

Number of bases with 1 error (B)=234/F

### [Example 11] Direct Verification of the Sequence Error Removal Effect by E. coli Transformation

We tested whether the error removal reaction prior to DNA amplification by a mismatch repair-related enzyme group is also effective in a system in which E. coli are transformed directly without in vitro DNA amplification (i.e., DNA is amplified in E. coli).

The L-GFP fragment (1.4 kb) was prepared using the primer pair of primers 8 and 9 shown in Table 27 and the R-GFP fragment (1.7 kb) using the primer pair of primers 10 and 11 shown in Table 28, respectively, by PCR using pUPGFP obtained in Example 8 as a template. In Tables 27 and 28, uppercase letters indicate base substitutions. The uppercase letter U in primers 9-11 indicates dU.

TABLE 27

| For L-GFP fragment | | SEQ ID NO. |
|---|---|---|
| Primer 8 | ataatctcaUgaccaaaaUcccttaacgU | 79 |
| Primer 9 | agcatUgaacaccataggUcagagtagUgac | 80 |
| Primer 9′ | agcatUgaacaccCtaggUcagagtagUgac | 81 |

TABLE 28

| For R-GFP fragment | | SEQ ID NO. |
|---|---|---|
| Primer 10 | actactcUgacctatggUgttcaatgcU | 82 |
| Primer 11 | acgttaagggattUtggtcaUgagattaUc | 83 |

For the introduction of artificial sequence error, primer 9 was changed to primer 9′ and the L-GFPmut fragment, a single base substitution of the L-GFP fragment, was prepared in the same way. L-GFP fragment and R-GFP fragments were designed to have homologous ends of about 30 base pairs with each other. Using these fragments, circular DNA was obtained by assembly and cyclization by the RA assembly reaction as in Example 1 (100% Match). Separately, half of the L-GFP fragments were replaced with L-GFPmut fragments, which were then cyclized in the RA assembly reaction to obtain circular DNA in the same manner (50% Mismatch). The single nucleotide substitution in the L-GFPmut fragment is located at the homologous end, and the RA assembly results in a GA mismatch. This single nucleotide substitution is designed to cause a nonsense mutation in the gfp gene, resulting in GFP negative E. coli transformed colonies.

For each 1 μL of circular DNA, the same technique as in Example 8 was used with the mismatch repair-related enzyme group SLD (300 nM MutS, 300 nM MutL, 15 nM UvrD) plus 0, 2 or 20 mU/μL of ExoVII, or without mismatch repair-related enzyme group (None) reaction solution (5 μL total), and mismatch removal was performed at 37° C. for 30 min. Using 1 μL of the obtained reaction product, the E. coli DH5a strain was directly transformed without DNA amplification reaction in a cell-free system, and the percentage of E. coli colonies that were GFP negative was counted using the same method as in Example 8. The counting results are shown in FIG. 11.

As shown in FIG. 11, the percentage of GFP negative colonies at 50% mismatch was suppressed by the reaction of mismatch repair-related enzymes SLD before DNA amplification, and this suppression effect was further enhanced by ExoVII. The results indicate that the mismatch repair enzymes SLD or SLDE can remove sequence errors before DNA amplification without in vitro amplification reaction.

### [Example 12] Examination of PCR Amplification Reaction after Sequence Error Removal Reaction

DNA assembly products containing sequence errors were subjected to an error removal reaction prior to DNA amplification, and then PCR amplification was performed to examine whether the error removal effect was observed in the amplified products.

The same technique as in Example 4 was used for the RA assembly reaction of the UPL fragment with the UPR fragment (100% Match). Separately, half of the UPR fragments were replaced with UPR-U_GT fragment (Table 11) containing an artificial error in the Nrul recognition sequence site,. which were then cyclized in the RA assembly reaction to obtain circular DNA in the same manner (50% Mismatch).

Each DNA assembly product was subjected to an error removal reaction with a mismatch repair-related enzyme group prior to DNA amplification as in Example 4. Error removal reactions were performed with (+) or without (−) the mismatch repair-related enzyme group, using 300 nM MutS, 300 nM MutL, 15 nM UvrD, and 100 mU/μL ExoVII (SLDE) as the mismatch repair-related enzyme group.

Next, a Supercoiling and Repair Reaction (SCR) was performed according to the method of Fujita et al. (Fujita et al., ACS Synth. Biol., (2022) vol. 11, p. 3088-3099) to repair the single-stranded gap in the DNA assembly site. PCR reactions using primers 12 and 13 shown in Table 29 were performed on the gap repair product to amplify a 367 base pair fragment containing the assembly site.

TABLE 29

| For amplifying fragment with artificial error | | SEQ ID NO. |
|---|---|---|
| Primer 12 | ACCGAGATCTACACGCGTAAG | 84 |
| Primer 13 | CATCTGTGCGGTATTTCACACC | 85 |

To detect the percentage of artificial error contained in the amplified product, cleavage with the restriction enzyme Nrul was performed and the percentage of cleavage was detected by agarose gel electrophoresis. As mentioned above, since the artificial errors are located in the Nrul recognition site, residual errors are detected as a band of 367 base pairs of Nrul resistance. The results are shown in FIG. 12.

As shown in FIG. 12, for the 100% Match sample, almost all PCR products were cleaved by Nrul in both conditions, and bands of 187 and 180 base pairs were detected (Correct). On the other hand, for the 50% Mismatch sample, about half of the Nrul-resistant bands (Error) remained, and this percentage was suppressed to 18% in the sample treated with the mismatch repair-related enzyme group SLDEs. It was shown that the treatment using mismatch repair enzyme group SLDE before DNA amplification is effective for removing sequence errors in the assembly product, even when using a PCR-based amplification system.

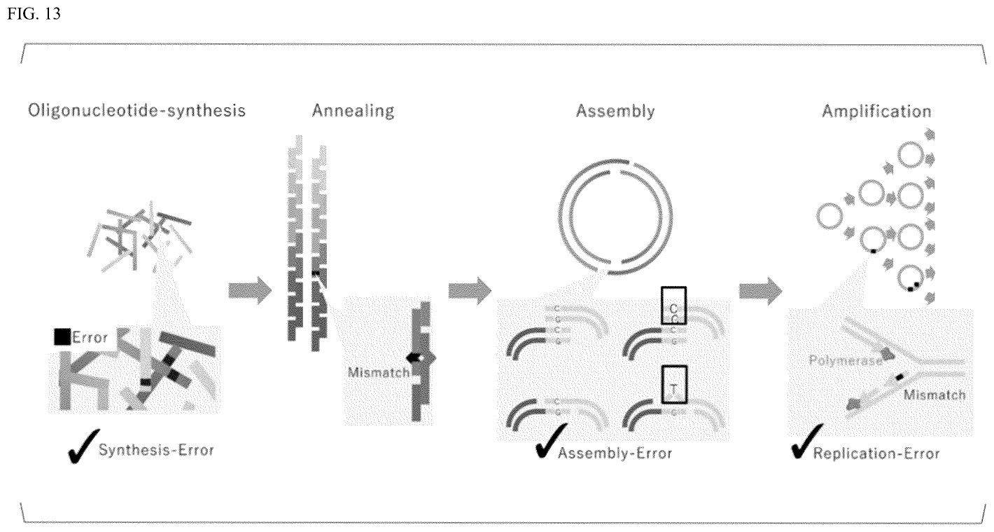

The effects of mismatch repair-related enzymes on a variety of sequence errors identified from the above examples are summarized in FIG. 13. The method of the invention using mismatch repair-related enzyme group was effective in removing sequence errors in a variety of sequence errors, including (a) sequence errors generated during oligonucleotide synthesis and annealing of oligonucleotides with such sequence errors, (b) sequence errors generated during double-stranded DNA assembly, and (c) replication errors during DNA amplification.

1. A method for producing double-stranded DNA, comprising:
(1) providing a double-stranded DNA mixture containing double-stranded DNA with sequence error and double-stranded DNA without sequence error;
(2) adding a mismatch repair-related enzyme group to the double-stranded DNA mixture, wherein the mismatch repair-related enzyme group comprising MutS and MutL; and
(3) subjecting the double-stranded DNA mixture to a double-stranded DNA amplification reaction.

2. The method according to claim 1, wherein the mismatch repair-related enzyme group further comprises enzymes selected from MutH, UvrD, and a combination of UvrD and a single strand-specific exonuclease.

3. The method according to claim 1, wherein said (2) comprises acting the mismatch repair-related enzyme group on double-stranded DNA with sequence error in the double-stranded DNA mixture.

4. The method according to claim 1, wherein the mismatch repair-related enzyme group further comprises UvrD and single strand-specific exonuclease, and the single strand-specific exonuclease is ExoVII.

5. The method according to claim 1, wherein the double-stranded DNA amplification reaction of said (3) is a cell-free amplification reaction, and comprising acting the mismatch repair-related enzyme group on the double-stranded DNA with sequence error in the double-stranded DNA mixture.

6. The method according to claim 5, wherein the mismatch repair-related enzyme group further comprises MutH.

7. A method for producing double-stranded DNA, comprising:
(1) providing a double-stranded DNA mixture containing double-stranded DNA with sequence error and double-stranded DNA without sequence error;
(2) adding a mismatch repair-related enzyme group to the double-stranded DNA mixture, wherein the mismatch repair-related enzyme group comprising MutS and a single strand-specific exonuclease; and
(3) subjecting the double-stranded DNA mixture to a double-stranded DNA amplification reaction,
wherein the double-stranded DNA amplification reaction of said (3) is a cell-free amplification reaction, and comprising acting the mismatch repair-related enzyme group on the double-stranded DNA with sequence error in the double-stranded DNA mixture.

8. The method according to claim 7, wherein the mismatch repair-related enzyme group further comprises one or more enzymes selected from MutL and MutH.

9. The method according to claim 7, wherein the single strand-specific exonuclease is exonuclease I.

10. The method according to claim 1, wherein the amplification reaction is conducted at a temperature of 65° C. or lower.

11. The method according to claim 1, wherein said step (1) comprises:
obtaining the double-stranded DNA with sequence error by mis-hybridizing a part or all of the single strand portions in at least one of the combinations selected from:
a combination of single-stranded DNA and its complementary strand single-stranded DNA,
a combination of double-stranded DNA with a single-stranded portion and single-stranded DNA with a sequence complementary to at least a part of the single-stranded portion, and
a combination of double-stranded DNA with a single-stranded portion and double-stranded DNA with a single-stranded portion having a sequence complementary to at least a part of the single-stranded portion,
or
obtaining the double-stranded DNA with sequence error by hybridizing a part or all of the single strand portions in at least one of the combinations selected from:
a combination of single-stranded DNA and its complementary strand single-stranded DNA, wherein at least one of the single-stranded DNA has sequence error,
a combination of double-stranded DNA with a single-stranded portion and single-stranded DNA with a sequence complementary to at least a part of the single-stranded portion, wherein at least one of the double-stranded DNA and the single-stranded DNA has sequence error, and
a combination of double-stranded DNA with a single-stranded portion and double-stranded DNA with a single-stranded portion having a sequence complementary to at least a part of the single-stranded portion, wherein at least one of the DNAs has sequence error.

12. A method for producing double-stranded DNA using a double-stranded DNA amplification reaction, comprising:
subjecting a reaction solution containing mismatch repair-related enzyme group and double-stranded DNA to the double-stranded DNA amplification reaction, wherein
the mismatch repair-related enzyme group comprises: MutS; and MutL and/or single strand-specific exonuclease, and
the double-stranded DNA amplification reaction is a cell-free amplification reaction conducted at a temperature of 80° C. or lower.

13. The method according to claim 12, wherein the mismatch repair-related enzyme group further comprises MutH.

14. The method according to claim 12, wherein the double-stranded DNA subjected to the amplification reaction is double-stranded DNA obtained by hybridizing a part or all of the single-stranded portion of a combination selected from:
a combination of single-stranded DNA and its complementary strand single-stranded DNA,
a combination of double-stranded DNA with a single-stranded portion and double-stranded DNA with single-stranded portion with a sequence complementary to at least a part of said single-stranded portion, and
a combination of double-stranded DNA with a single-stranded portion and double-stranded DNA with a sequence complementary to at least a part of the single-stranded portion.

15. The method according to claim 1, wherein
the double-stranded DNA subjected to the amplification reaction is a circular double-stranded DNA having a replication origin sequence capable of binding to an enzyme with DnaA activity, and
the double-stranded DNA amplification reaction is an RCR amplification reaction.

16. A double-stranded DNA obtained by the method according to claim 1.

17. A kit for producing circular double-stranded DNA, comprising:
(A)
MutS,
MutL,
UvrD,
a single strand-specific exonuclease,
a first enzyme group that catalyzes replication of circular DNA;
a second enzyme group that catalyzes an Okazaki fragment maturation and synthesizes two sister circular DNAs constituting a catenane; and
a third enzyme group that catalyzes a separation of two sister circular DNAs;
(B)
MutS,
MutL,
MutH and/or a single strand-specific exonuclease,
a first group of enzymes catalyzing the replication of circular DNA;
a second group of enzymes catalyzing Okazaki fragment joining reactions to synthesize two sister circular DNAs forming a catenane, and
a third group of enzymes catalyzing the separation of the two sister circular DNAs; or
(C).
MutS,
a single strand-specific exonuclease,
a first enzyme group that catalyzes replication of circular DNA,
a second enzyme group that catalyzes an Okazaki fragment maturation and synthesizes two sister circular DNAs constituting a catenane, and
a third enzyme group that catalyzes a separation of two sister circular DNAs.

18-19. (canceled)


SEQUENCE LISTING (85 sequences)

SEQ ID NO: 1 - DNA, 1370 bp, synthetic construct
  source 1..1370: mol_type = other DNA
ggcttagtgaaacaaagcttcagtcccgtactctacgcgccagagggaaattcacctggcgcgtatttttgttcgcggcttagctacggctgagcacgccgcggcgatagagattacgtttaatgcgcgttaaaccaggttttggcttacgcggttcgtccaggcttgccaggacaatctccagcacacgctctgcgacatcgcggtgacgttgagccactgccagcaccggacactgtaagaagtcgagcagttcgttatcgccaaaggtggcaattgccaggtcagaaggcagtttgccgtcgcgacgcagcgtgacatccatcactccttgcaacaacgcaaacgacgttgtgaacagcgcctgcggcatcggatgcgtttccagccatttttcgaataactgggcagccgcctcccgctcatagctgttggcatacaggaaatgcacttcgcgcggatcatctttccaggcagtacggaaaccttgttcacgcaggaagctgacagaaagctccggtagcgcaccaagataaagcaccgtctcggcgggaaacttacgtaactcttccgccagcatttcggcatcatcctgatcggcaccaaccacgctggtgaagtgttcacgatcgagggcgcggtccagcgcgacaatcgggaacgggtcgttagcccagcgttgataaaaaggatgctcaggaggcaacgacgtcgaaacaataatggcatcaacctgacgctgtaaaaggtgctcaatgcaccgcatttcgttgtctggctgatcttctgagcaggcaatcagcagttgataaccccgttgccgcgcctggcgttcaagatagttagcgatgcgggtatagctggtgttctccagatcggggatcacaagaccaatagaacgtgtgcgtccagcacgaagcccagctgccacggcgttcgggtggtaattgtgctcacgcaccacagccatgactttttcaacggttttgtcgctcacacggtattgcttcgctttgccgttaataacatagcttgcagtggtccgcgacactcccgccagccgagcgatttcatccagtttcacaattgccccttgcgtaaaatgtaaaaaccatagccattgtactggtatgggttaaattcattaacatctaagcgcagaaaattaactgcggcaactgcttttattccaggttcccactgatttcgcaaaaaaagcccaacgtgacatgttgggctgtaaattgcgcattgagatcattctcaacgcattattttatcgccgcgcgaaagtccgaccacaccagagcgagcaacctccacaattttcgccacatcgcgaatcgatgctttatcaaacgtgcgcacag

SEQ ID NO: 2 - DNA, 1316 bp, synthetic construct
  source 1..1316: mol_type = other DNA
gccacatcgcgaatcgatgctttatcaaacgtgcgcacagtccatccagagggctttacagtgtacatatcaacccatattcggcttgagaaagacctgagtatgttgtaactaaagggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtataacttcgtatagcatacattatacgaagttatctttagttacaacatactccattcttctgccggatcttcttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgctcattcccttctttatcgggttacagaaccggtttacgcagtttcggcttagtgaaacaaagcttcagtcccgtactctacgcgc

SEQ ID NO: 3 - DNA, 40 bp, synthetic construct
  source 1..40: mol_type = other DNA
ggcttagtgaaacaaagcttcagtcccgtactctacgcgc

SEQ ID NO: 4 - DNA, 40 bp, synthetic construct
  source 1..40: mol_type = other DNA
ggcttagtgaaacagagcttcagtcccgtactctacgcgc

SEQ ID NO: 5 - DNA, 651 bp, synthetic construct
  source 1..651: mol_type = other DNA
gtgctggatctgcccggagatccccaccgccaggtaccttaaaagcttcagcatcaggttgcatcggatgcgtttccagccatttttcgaataactgggcagccgcctcccgctcatagctgttggcatacaggaaatgcacttcgcgcggatcatctttccaggcagtacggaaaccttgttcacgcaggaagctgacagaaagctccggtagcgcaccaagataaagcaccgtctcggcgggaaacttacgtaactcttccgccagcatttcggcatcatcctgatcggcaccaaccacgctggtgaagtgttcacgatcgagggcgcggtccagcgcgacaatcgggaacgggtcgttagcccagcgttgataaaaaggatgctcaggaggcaacgacgtcgaaacaataatggcatcaacctgacgctgtaaaaggtgctcaatgcaccgcatttcgttgtctggctgatcttctgagcaggcaatcagcagttgataaccccgttgccgcgcctggcgttcaagatagttagcgatgcgggtatagctggtgttctccagatcggggatcacaagaccaatagaacgtgtgcgtccagcacgaagcccagctgccacggcgttcgggtggtaattgtgctcacgca

SEQ ID NO: 6 - DNA, 1927 bp, synthetic construct
  source 1..1927: mol_type = other DNA
cgaagcccagctgccacggcgttcgggtggtaattgtgctcacgcatttatcaaacgtgcgcacagtccatccagagggctttacagtgtacatatcaacccatattcggcttgagaaagacctgagtatgttgtaactaaagggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtataacttcgtatagcatacattatacgaagttatctttagttacaacatactccattcttctgccggatcttcttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgctcattcccttctttatcgggttacagaaccggtttacgcagtttcggcttagtgaaacaaagaaaagtgtttgttaaatcggcaattcatcgccgcatgtaacaaaataatgcaaaaacgcgctgtcgatatcacttaattattgtgaatttcagctggtgagttaattaacgggcgggtttaaaagcatttatcaataatagtcaggaatagtcttatttactttaagcatattgatgtccagtcccgtactctacgcgccagagggaaattcacctggcgcgtatttttgttcgcggcttagctacggctgagcacgccgcggcgatagagattacgtttaatgcgcgttaaaccaggttttggcttacgcggttcgtccaggcttgccaggacaatctccagcacacgctctgcgacatcgcggtgacgttgagccactgccagcaccggacactgtaagaagtcgagcagttcgttatcgccaaaggtggcaattgccaggtcagaaggcagtttgccgtcgcgacgcagcgtgacatccatcactccttgcaacaacgcaaacgacgttgtgaacagcgcctgcgagccgcctcccgctcatagcgtgctggatctgcccggagatccccaccgccaggtaccttaaaagcttcagcatcaggtt

SEQ ID NO: 7 - DNA, 60 bp, synthetic construct
  source 1..60: mol_type = other DNA
gtgctggatctgcccggagatccccaccgccaggtaccttaaaagcttcagcatcaggtt

SEQ ID NO: 8 - DNA, 60 bp, synthetic construct
  source 1..60: mol_type = other DNA
gtgctggatctgcccggagatccccaccgccaggtaccttaaaagcctcagcatcaggtt

SEQ ID NO: 9 - DNA, 60 bp, synthetic construct
  source 1..60: mol_type = other DNA
gtgctggatctgcccggagatccccaccgccaggtaccttaaaagcgtcagcatcaggtt

SEQ ID NO: 10 - DNA, 60 bp, synthetic construct
  source 1..60: mol_type = other DNA
gtgctggatctgcccggagatccccaccgccaggtaccttaacagcttcagcatcaggtt

SEQ ID NO: 11 - DNA, 60 bp, synthetic construct
  source 1..60: mol_type = other DNA
gtgctggatctgcccggagatccccaccgccaggtaccttaagagcttcagcatcaggtt

SEQ ID NO: 12 - DNA, 60 bp, synthetic construct
  source 1..60: mol_type = other DNA
gtgctggatctgcccggagatccccaccgccaggtaccttaaaagcatcagcatcaggtt

SEQ ID NO: 13 - DNA, 60 bp, synthetic construct
  source 1..60: mol_type = other DNA
gtgctggatctgcccggagatccccaccgccaggtaccttaaaaccttcagcatcaggtt

SEQ ID NO: 14 - DNA, 60 bp, synthetic construct
  source 1..60: mol_type = other DNA
gtgctggatctgcccggagatccccaccgccaggtaccttaatagcttcagcatcaggtt

SEQ ID NO: 15 - DNA, 60 bp, synthetic construct
  source 1..60: mol_type = other DNA
gtgctggatctgcccggagatccccaccgccaggtaccttaaaaggttcagcatcaggtt

SEQ ID NO: 16 - DNA, 41 bp, synthetic construct
  source 1..41: mol_type = other DNA
ggcttagtgaaacaaagacttcagtcccgtactctacgcgc

SEQ ID NO: 17 - DNA, 41 bp, synthetic construct
  source 1..41: mol_type = other DNA
ggcttagtgaaacaaagccttcagtcccgtactctacgcgc

SEQ ID NO: 18 - DNA, 41 bp, synthetic construct
  source 1..41: mol_type = other DNA
ggcttagtgaaacaaaggcttcagtcccgtactctacgcgc

SEQ ID NO: 19 - DNA, 41 bp, synthetic construct
  source 1..41: mol_type = other DNA
ggcttagtgaaacaaagtcttcagtcccgtactctacgcgc

SEQ ID NO: 20 - DNA, 2752 bp, synthetic construct
  source 1..2752: mol_type = other DNA
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgataaaaaaacagcctccgaaaggaggctgtaggcaaatattctagtatgttgtacctaaaggaaatgagctgacagatcgtgcgatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatccctttaggtacaacatactgtttgggattcactgagttttaaatcaatctaaagtatatatgagtaaacttggtctgacagttaccaatgcttatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgtcaggaagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaacggtctccaatgatacggcgaccaccgagatctacacgcgtaagatcgtcggcagcgtcagatgtgtataagagacagtgttcggtattggtgcagcggttctggtttatttcgggattaaaaaactgaaaaaagggcggggcagttcctggttacgtgacctgatttactggtatatgccaacagccctgctgcgcggttcgcgacataatgttcccgat

SEQ ID NO: 21 - DNA, 2026 bp, synthetic construct
  source 1..2026: mol_type = other DNA
aacagccctgctgcgcggttcgcgacataatgttcccgattcgtgtttccggcagtggattaaataaaactgctgtctcttatacacatctccgagcccacgagacaagaggcaatctcgtatgccgtcttctgcttgcgagaccatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccccggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatccggggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcagg

SEQ ID NO: 22 - DNA, 40 bp, synthetic construct
  source 1..40: mol_type = other DNA
aacagccctgctgcgcggttcgcgacataatgttcccgat

SEQ ID NO: 23 - DNA, 40 bp, synthetic construct
  source 1..40: mol_type = other DNA
  misc_feature 21: note = n is a, c, g, or t
aacagccctgctgcgcggttngcgacataatgttcccgat

SEQ ID NO: 24 - DNA, 42 bp, synthetic construct
  source 1..42: mol_type = other DNA
  modified_base 9: mod_base = OTHER; note = deoxyuridine (dU)
  modified_base 19: mod_base = OTHER; note = deoxyuridine (dU)
  modified_base 28: mod_base = OTHER; note = deoxyuridine (dU)
  modified_base 40: mod_base = OTHER; note = deoxyuridine (dU)
aacagcccdgcagcgcggdtcgcgacadaatgttcccgadtc

SEQ ID NO: 25 - DNA, 47 bp, synthetic construct
  source 1..47: mol_type = other DNA
  modified_base 12: mod_base = OTHER; note = deoxyuridine (dU)
  modified_base 24: mod_base = OTHER; note = deoxyuridine (dU)
  modified_base 42: mod_base = OTHER; note = deoxyuridine (dU)
atgttctttccdgcgttatccccdgattctgtggataaccgdattac

SEQ ID NO: 26 - DNA, 42 bp, synthetic construct
  source 1..42: mol_type = other DNA
  modified_base 9: mod_base = OTHER; note = deoxyuridine (dU)
  modified_base 19: mod_base = OTHER; note = deoxyuridine (dU)
  modified_base 28: mod_base = OTHER; note = deoxyuridine (dU)
  modified_base 40: mod_base = OTHER; note = deoxyuridine (dU)
aacagcccdgcagcgcggdtagcgacadaatgttcccgadtc

SEQ ID NO: 27 - DNA, 43 bp, synthetic construct
  source 1..43: mol_type = other DNA
  modified_base 8: mod_base = OTHER; note = deoxyuridine (dU)
  modified_base 17: mod_base = OTHER; note = deoxyuridine (dU)
  modified_base 25: mod_base = OTHER; note = deoxyuridine (dU)
  modified_base 42: mod_base = OTHER; note = deoxyuridine (dU)
acggttadccacagaadcaggggadaacgcaggaaagaacadg

SEQ ID NO: 28 - DNA, 41 bp, synthetic construct
  source 1..41: mol_type = other DNA
  modified_base 16: mod_base = OTHER; note = deoxyuridine (dU)
  modified_base 29: mod_base = OTHER; note = deoxyuridine (dU)
  modified_base 40: mod_base = OTHER; note = deoxyuridine (dU)
atcgggaacattatgdcgcgaaccgcgcdgcagggctgtdg

SEQ ID NO: 29 - DNA, 40 bp, synthetic construct
  source 1..40: mol_type = other DNA
aacagccctgctgcgcggtttgcgacataatgttcccgat

SEQ ID NO: 30 - DNA, 42 bp, synthetic construct
  source 1..42: mol_type = other DNA
  modified_base 9: mod_base = OTHER; note = deoxyuridine (dU)
  modified_base 19: mod_base = OTHER; note = deoxyuridine (dU)
  modified_base 28: mod_base = OTHER; note = deoxyuridine (dU)
  modified_base 40: mod_base = OTHER; note = deoxyuridine (dU)
aacagcccdgcagcgcggdttgcgacadaatgttcccgadtc

SEQ ID NO: 31 - DNA, 2364 bp, synthetic construct
  source 1..2364: mol_type = other DNA
atggactcgtctactagcgcagcttaattaacctaggctgctgccaccgctgagcaataaataaaacgaaaggctcagtcgaaagactgggcctttcgttttatctgaaaggaggaactatatccggattggcgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagagagctgttgacagagggtcattttcacactataatgcagtgaatcccaaacagtatgttgtacctaaagggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtcagctcatttcctttaggtacaacatactagaatatttgcctacagcctcctttcggaggctgtttttttatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaattgacatccctatcagtgatagatataatatgcgcatcctctagaaataattttgtttaacttaagaaggagatatacc

SEQ ID NO: 32 - DNA, 53 bp, synthetic construct
  source 1..53: mol_type = other DNA
  modified_base 15: mod_base = OTHER; note = deoxyuridine (dU)
  modified_base 29: mod_base = OTHER; note = deoxyuridine (dU)
  modified_base 42: mod_base = OTHER; note = deoxyuridine (dU)
  modified_base 51: mod_base = OTHER; note = deoxyuridine (dU)
atggactcgtctacdagcgcagcttaatdaacctaggctgcdgccaccgcdga

SEQ ID NO: 33 - DNA, 39 bp, synthetic construct
  source 1..39: mol_type = other DNA
aacagccctgctgcgcggttcgcgcataatgttcccgat

SEQ ID NO: 34 - DNA, 39 bp, synthetic construct
  source 1..39: mol_type = other DNA
aacagccctgctgcgcggttcggacataatgttcccgat

SEQ ID NO: 35 - DNA, 39 bp, synthetic construct
  source 1..39: mol_type = other DNA
aacagccctgctgcgcggttccgacataatgttcccgat

SEQ ID NO: 36 - DNA, 146 bp, synthetic construct
  source 1..146: mol_type = other DNA
tttcgggattaaaaaactgaaaaaagggcggggcagttcctggttacgtgacctgatttactggtatatgccaacagccctgctgcgcggttcgcgacataatgttcccgattcgtgtttccggcagtggattaaataaaactgct

SEQ ID NO: 37 - DNA, 146 bp, synthetic construct
  source 1..146: mol_type = other DNA
tttcgggattaaaaaactgaaaaaagggcggggcagttcctggttacgtgacctgatttactggtatatgccaacagccctgctgcgcggtttgcgacataatgttcccgattcgtgtttccggcagtggattaaataaaactgct

SEQ ID NO: 38 - DNA, 147 bp, synthetic construct
  source 1..147: mol_type = other DNA
gaatcgggaacattatgtcgcgaaccgcgcagcagggctgttggcatataccagtaaatcaggtcacgtaaccaggaactgccccgcccttttttcagttttttaatcccgaaataaaccagaaccgctgcaccaataccgaacact

SEQ ID NO: 39 - DNA, 33 bp, synthetic construct
  source 1..33: mol_type = other DNA
  modified_base 6: mod_base = OTHER; note = deoxyuridine (dU)
  modified_base 18: mod_base = OTHER; note = deoxyuridine (dU)
  modified_base 32: mod_base = OTHER; note = deoxyuridine (dU)
gtgttdccggcagtggadtaaataaaactgcdg

SEQ ID NO: 40 - DNA, 34 bp, synthetic construct
  source 1..34: mol_type = other DNA
  modified_base 15: mod_base = OTHER; note = deoxyuridine (dU)
  modified_base 23: mod_base = OTHER; note = deoxyuridine (dU)
  modified_base 33: mod_base = OTHER; note = deoxyuridine (dU)
taaaccagaaccgcdgcaccaadaccgaacacdg

SEQ ID NO: 41 - DNA, 160 bp, synthetic construct
  source 1..160: mol_type = other DNA
gggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggt

SEQ ID NO: 42 - DNA, 160 bp, synthetic construct
  source 1..160: mol_type = other DNA
cattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatccgtaat

SEQ ID NO: 43 - DNA, 41 bp, synthetic construct
  source 1..41: mol_type = other DNA
  modified_base 6: mod_base = OTHER; note = deoxyuridine (dU)
  modified_base 14: mod_base = OTHER; note = deoxyuridine (dU)
  modified_base 23: mod_base = OTHER; note = deoxyuridine (dU)
  modified_base 40: mod_base = OTHER; note = deoxyuridine (dU)
gcgaadggcgcttdgcctggttdccggcaccagaagcggdg

SEQ ID NO: 44 - DNA, 41 bp, synthetic construct
  source 1..41: mol_type = other DNA
  modified_base 14: mod_base = OTHER; note = deoxyuridine (dU)
  modified_base 29: mod_base = OTHER; note = deoxyuridine (dU)
  modified_base 40: mod_base = OTHER; note = deoxyuridine (dU)
agtcacgacgttgdaaaacgacggccagdgaatccgtaadc

SEQ ID NO: 45 - DNA, 99 bp, synthetic construct
  source 1..99: mol_type = other DNA
caacaagaattgggacaactccagtgaaaagttcttctcctttgctcatggtatatctccttcttaagttaaacaaaattatttctagaggatgcgcat

SEQ ID NO: 46 - DNA, 99 bp, synthetic construct
  source 1..99: mol_type = other DNA
atgagcaaaggagaagaacttttcactggagttgtcccaattcttgttgaattagatggtgatgttaatgggcacaaattttctgtccgtggagagggt

SEQ ID NO: 47 - DNA, 100 bp, synthetic construct
  source 1..100: mol_type = other DNA
gtagtgcaaataaatttaagggtgagttttccgtttgtagcatcaccttcaccctctccacggacagaaaatttgtgcccattaacatcaccatctaatt

SEQ ID NO: 48 - DNA, 92 bp, synthetic construct
  source 1..92: mol_type = other DNA
gaaggtgatgctacaaacggaaaactcacccttaaatttatttgcactactggaaaactacctgttccgtggccaacacttgtcactactct

SEQ ID NO: 49 - DNA, 89 bp, synthetic construct
  source 1..89: mol_type = other DNA
gtttcatgtgatccggataacgggaaaagcattgaacaccataggtcagagtagtgacaagtgttggccacggaacaggtagttttcca

SEQ ID NO: 50 - DNA, 90 bp, synthetic construct
  source 1..90: mol_type = other DNA
gacctatggtgttcaatgcttttcccgttatccggatcacatgaaacggcatgactttttcaagagtgccatgcccgaaggttatgtaca

SEQ ID NO: 51 - DNA, 86 bp, synthetic construct
  source 1..86: mol_type = other DNA
cgtcttgtaggtcccgtcatctttgaaagatatagtgcgttcctgtacataaccttcgggcatggcactcttgaaaaagtcatgcc

SEQ ID NO: 52 - DNA, 93 bp, synthetic construct
  source 1..93: mol_type = other DNA
ggaacgcactatatctttcaaagatgacgggacctacaagacgcgtgctgaagtcaagtttgaaggtgatacccttgttaatcgtatcgagtt

SEQ ID NO: 53 - DNA, 100 bp, synthetic construct
  source 1..100: mol_type = other DNA
caagtttgtgtccaagaatgtttccatcttctttaaaatcaataccctttaactcgatacgattaacaagggtatcaccttcaaacttgacttcagcacg

SEQ ID NO: 54 - DNA, 100 bp, synthetic construct
  source 1..100: mol_type = other DNA
aaagggtattgattttaaagaagatggaaacattcttggacacaaacttgagtacaactttaactcacacaatgtatacatcacggcagacaaacaaaag

SEQ ID NO: 55 - DNA, 100 bp, synthetic construct
  source 1..100: mol_type = other DNA
gaaccatcttcaacgttgtggcgaattttgaagttagctttgattccattcttttgtttgtctgccgtgatgtatacattgtgtgagttaaagttgtact

SEQ ID NO: 56 - DNA, 100 bp, synthetic construct
  source 1..100: mol_type = other DNA
aatggaatcaaagctaacttcaaaattcgccacaacgttgaagatggttccgttcaactagcagaccattatcaacaaaatactccaattggcgatggcc

SEQ ID NO: 57 - DNA, 98 bp, synthetic construct
  source 1..98: mol_type = other DNA
aaaggacagattgtgtcgacaggtaatggttgtctggtaaaaggacagggccatcgccaattggagtattttgttgataatggtctgctagttgaacg

SEQ ID NO: 58 - DNA, 89 bp, synthetic construct
  source 1..89: mol_type = other DNA
ctgtccttttaccagacaaccattacctgtcgacacaatctgtcctttcgaaagatcccaacgaaaagcgtgaccacatggtccttctt

SEQ ID NO: 59 - DNA, 92 bp, synthetic construct
  source 1..92: mol_type = other DNA
ttaatacagctcatccataccatgtgtaatcccagcagcagttacaaactcaagaaggaccatgtggtcacgcttttcgttgggatctttcg

SEQ ID NO: 60 - DNA, 93 bp, synthetic construct
  source 1..93: mol_type = other DNA
gagtttgtaactgctgctgggattacacatggtatggatgagctgtattaaatggactcgtctactagcgcagcttaattaacctaggctgct

SEQ ID NO: 61 - DNA, 95 bp, synthetic construct
  source 1..95: mol_type = other DNA
aagaattgggacaactccagtgaaaagttcttctcctttgctcatggtatatctccttcttaagttaaacaaaattatttctagaggatgcgcat

SEQ ID NO: 62 - DNA, 95 bp, synthetic construct
  source 1..95: mol_type = other DNA
atgagcaaaggagaagaacttttcactggagttgtcccaattcttgttgaattagatggtgatgttaatgggcacaaattttctgtccgtggaga

SEQ ID NO: 63 - DNA, 96 bp, synthetic construct
  source 1..96: mol_type = other DNA
aataaatttaagggtgagttttccgtttgtagcatcaccttcaccctctccacggacagaaaatttgtgcccattaacatcaccatctaattcaac

SEQ ID NO: 64 - DNA, 95 bp, synthetic construct
  source 1..95: mol_type = other DNA
gggtgaaggtgatgctacaaacggaaaactcacccttaaatttatttgcactactggaaaactacctgttccgtggccaacacttgtcactactc

SEQ ID NO: 65 - DNA, 95 bp, synthetic construct
  source 1..95: mol_type = other DNA
ttcatgtgatccggataacgggaaaagcattgaacaccataggtcagagtagtgacaagtgttggccacggaacaggtagttttccagtagtgca

SEQ ID NO: 66 - DNA, 95 bp, synthetic construct
  source 1..95: mol_type = other DNA
tgacctatggtgttcaatgcttttcccgttatccggatcacatgaaacggcatgactttttcaagagtgccatgcccgaaggttatgtacaggaa

SEQ ID NO: 67 - DNA, 95 bp, synthetic construct
  source 1..95: mol_type = other DNA
cagcacgcgtcttgtaggtcccgtcatctttgaaagatatagtgcgttcctgtacataaccttcgggcatggcactcttgaaaaagtcatgccgt

SEQ ID NO: 68 - DNA, 96 bp, synthetic construct
  source 1..96: mol_type = other DNA
cgcactatatctttcaaagatgacgggacctacaagacgcgtgctgaagtcaagtttgaaggtgatacccttgttaatcgtatcgagttaaagggt

SEQ ID NO: 69 - DNA, 96 bp, synthetic construct
  source 1..96: mol_type = other DNA
actcaagtttgtgtccaagaatgtttccatcttctttaaaatcaataccctttaactcgatacgattaacaagggtatcaccttcaaacttgactt

SEQ ID NO: 70 - DNA, 96 bp, synthetic construct
  source 1..96: mol_type = other DNA
attgattttaaagaagatggaaacattcttggacacaaacttgagtacaactttaactcacacaatgtatacatcacggcagacaaacaaaagaat

SEQ ID NO: 71 - DNA, 96 bp, synthetic construct
  source 1..96: mol_type = other DNA
aaccatcttcaacgttgtggcgaattttgaagttagctttgattccattcttttgtttgtctgccgtgatgtatacattgtgtgagttaaagttgt

SEQ ID NO: 72 - DNA, 96 bp, synthetic construct
  source 1..96: mol_type = other DNA
ggaatcaaagctaacttcaaaattcgccacaacgttgaagatggttccgttcaactagcagaccattatcaacaaaatactccaattggcgatggc

SEQ ID NO: 73 - DNA, 95 bp, synthetic construct
  source 1..95: mol_type = other DNA
gacagattgtgtcgacaggtaatggttgtctggtaaaaggacagggccatcgccaattggagtattttgttgataatggtctgctagttgaacgg

SEQ ID NO: 74 - DNA, 95 bp, synthetic construct
  source 1..95: mol_type = other DNA
cctgtccttttaccagacaaccattacctgtcgacacaatctgtcctttcgaaagatcccaacgaaaagcgtgaccacatggtccttcttgagtt

SEQ ID NO: 75 - DNA, 96 bp, synthetic construct
  source 1..96: mol_type = other DNA
ttaatacagctcatccataccatgtgtaatcccagcagcagttacaaactcaagaaggaccatgtggtcacgcttttcgttgggatctttcgaaag

SEQ ID NO: 76 - DNA, 97 bp, synthetic construct
  source 1..97: mol_type = other DNA
tgtaactgctgctgggattacacatggtatggatgagctgtattaaatggactcgtctactagcgcagcttaattaacctaggctgctgccaccgct

SEQ ID NO: 77 - DNA, 44 bp, synthetic construct
  source 1..44: mol_type = other DNA
  modified_base 15: mod_base = OTHER; note = deoxyuridine (dU)
  modified_base 29: mod_base = OTHER; note = deoxyuridine (dU)
  modified_base 42: mod_base = OTHER; note = deoxyuridine (dU)
atggactcgtctacdagcgcagcttaatdaacctaggctgcdgc

SEQ ID NO: 78 - DNA, 52 bp, synthetic construct
  source 1..52: mol_type = other DNA
  modified_base 16: mod_base = OTHER; note = deoxyuridine (dU)
  modified_base 37: mod_base = OTHER; note = deoxyuridine (dU)
  modified_base 50: mod_base = OTHER; note = deoxyuridine (dU)
ggtatatctccttctdaagttaaacaaaattatttcdagaggatgcgcadat

SEQ ID NO: 79 - DNA, 29 bp, synthetic construct
  source 1..29: mol_type = other DNA
  modified_base 10: mod_base = OTHER; note = deoxyuridine (dU)
  modified_base 19: mod_base = OTHER; note = deoxyuridine (dU)
  modified_base 29: mod_base = OTHER; note = deoxyuridine (dU)
ataatctcadgaccaaaadcccttaacgd

SEQ ID NO: 80 - DNA, 31 bp, synthetic construct
  source 1..31: mol_type = other DNA
  modified_base 6: mod_base = OTHER; note = deoxyuridine (dU)
  modified_base 19: mod_base = OTHER; note = deoxyuridine (dU)
  modified_base 28: mod_base = OTHER; note = deoxyuridine (dU)
agcatdgaacaccataggdcagagtagdgac

SEQ ID NO: 81 - DNA, 31 bp, synthetic construct
  source 1..31: mol_type = other DNA
  modified_base 6: mod_base = OTHER; note = deoxyuridine (dU)
  modified_base 19: mod_base = OTHER; note = deoxyuridine (dU)
  modified_base 28: mod_base = OTHER; note = deoxyuridine (dU)
agcatdgaacaccctaggdcagagtagdgac

SEQ ID NO: 82 - DNA, 28 bp, synthetic construct
  source 1..28: mol_type = other DNA
  modified_base 8: mod_base = OTHER; note = deoxyuridine (dU)
  modified_base 18: mod_base = OTHER; note = deoxyuridine (dU)
  modified_base 28: mod_base = OTHER; note = deoxyuridine (dU)
actactcdgacctatggdgttcaatgcd

SEQ ID NO: 83 - DNA, 30 bp, synthetic construct
  source 1..30: mol_type = other DNA
  modified_base 14: mod_base = OTHER; note = deoxyuridine (dU)
  modified_base 21: mod_base = OTHER; note = deoxyuridine (dU)
  modified_base 29: mod_base = OTHER; note = deoxyuridine (dU)
acgttaagggattdtggtcadgagattadc

SEQ ID NO: 84 - DNA, 21 bp, synthetic construct
  source 1..21: mol_type = other DNA
accgagatctacacgcgtaag

SEQ ID NO: 85 - DNA, 22 bp, synthetic construct
  source 1..22: mol_type = other DNA
catctgtgcggtatttcacacc